(19) United States
(12) Patent Application Publication
(10) Pub. No.: US 2013/0102490 A1
(43) Pub. Date: Apr. 25, 2013

(54) METHOD FOR COUNTING CHROMATID COPY NUMBERS IN A SINGLE CELL

(71) Applicants: Angelika H. Daser, Frankfurt (DE); Paul H. Dear, Cambridge (GB)

(72) Inventors: Angelika H. Daser, Frankfurt (DE); Paul H. Dear, Cambridge (GB)

(73) Assignee: MEDICAL RESEARCH COUNCIL, Swindon, Wiltshire (GB)

(21) Appl. No.: 13/695,237

(22) PCT Filed: May 4, 2011
(86) PCT No.: PCT/IB2011/051979

§ 371 Date: Jan. 9, 2013

Related U.S. Application Data
(60) Provisional application No. 61/395,414, filed on May 13, 2010.

(30) Foreign Application Priority Data
May 5, 2010 (GB) 1007522.4

(51) Int. Cl. C12Q 1/68

(52) U.S. Cl. CPC C12Q 1/686
USPC 506/9; 536/24.33

(57) ABSTRACT

The present invention provides a method for counting the absolute copy number of a nucleic acid sequence in a cell, which comprises the following steps: (i) dividing a lysate of the cell or a lysate of a sample of the cell into a plurality aliquots: (ii) providing conditions suitable for the amplification of the nucleic acid sequence in each aliquot: (iii) counting the number of aliquots in which the nucleic acid was amplified in step (ii) and directly deducing the copy number of the nucleic acid sequence in a cell. The method may be used to count chromatid copy number, for example to investigate the ploidy of a cell such as an oocyte or an embryo-derived cell.

The present invention relates to a method for counting the copy number of a nucleic acid sequence in a cell, for example a single cell. The method may be used for counting the copy number of a chromatid in a cell. The ploidy status of the cell may be investigated by counting the copy number of chromatids for each chromosome in the cell.

### BACKGROUND TO THE INVENTION

In vitro fertilisation (IVF) is a process by which egg cells are fertilised by sperm in vitro and the resultant zygote transferred to the patient's uterus with the intent to establish a successful pregnancy. The first human baby resulting from an IVF procedure was born in 1978, and since then IVF has become a major treatment for infertility when other methods of assisted reproductive technology have failed.

Despite the fact that IVF procedures are now relatively routine in many countries, clinical pregnancy rates and baby take home rates after IVF are still poor. Chromosomal abnormalities, which usually cause miscarriage, result predominantly from anomalies during female meiosis. A major factor is advanced maternal age and its impact on the quality of the oocyte. It is known that the decreasing fertility of older women is mainly caused by age-dependent increases of aneuploidies in oocytes (and embryos). Selection of euploid oocytes is thus an attractive strategy to increase the number of live births following IVF procedures.

The ploidy status of oocytes can be indirectly investigated by analysing the chromosome content in polar bodies (PB) I and II. Polar bodies are results of the first and second meiotic division before and after fertilisation (see FIG. 1).

Errors in meiotic divisions occur frequently and increase with maternal age; mechanisms are chromosome non-disjunction and early sister chromatid separation with higher frequency in meiosis I. Depending on the mechanism of malsegregation various chromosomal constellations can occur in oocyte and PB as exemplified for meiosis I (see FIG. 2).

At a slightly lower frequency, errors occur also during meiosis II due to non-disjunction and chromatid malsegregation. In order to provide a true picture of the chromosome content of the ooctye, ideally one would need to investigate the chromosome content of PB I and II for all chromosomes at the resolution of chromatids.

Although preimplantation genetic diagnostic (PGD) procedures are known, all are associated with shortcomings. Fluorescence in situ hybridisation (FISH) is sometimes used with different colour fluorescence for each chromosome. So far, this technique has been used with a maximum of 12 chromosomes. As only a subset of chromosomes is investigated, this leaves non-stained chromosome aneuploidies undetected. Array-based methods have also been used, but they have a sensitivity which does not always resolve below the chromosome level, meaning that they may not detect sister chromatid malsegregation which can occur in both meioisis I and II leading to aneuploid embryos. Moreover the array-based methods take at least 48 hours, thus making embryo freezing and implantation in a consecutive cycle necessary.

There is thus a need for improved methods for investigating the ploidy status of oocytes.

### SUMMARY OF ASPECTS OF THE INVENTION

The present inventors have developed a method which determines the absolute copy numbers of nucleic acid sequences, such as genomic markers, within a single cell. The copy numbers of nucleic acid sequences may, for example, represent the total number of each type of chromatid in the cell.

The method has been validated by chromatid counting in a haploid polar body and a diploid fibroblast at telophase, to assess the number of chromatids and through this the ploidy status of such single cells.

Thus in a first aspect, the present invention provides a method for counting the absolute copy number of a nucleic acid sequence in a cell, which comprises the following steps:
(i) dividing a lysate of the cell or a lysate of a sample of the cell into a plurality of aliquots;
(ii) providing conditions suitable for the amplification of the nucleic acid sequence in each aliquot;
(iii) counting the number of aliquots in which the nucleic acid was amplified in step (ii) and thus the copy number of the nucleic acid sequence in the cell.

In step (i), the lysate may be divided into at least 8 aliquots per cell used to make the lysate. Where the cell is diploid, the lysate may be divided into at least 16 aliquots per cell.

Where a sample of the cell is used in step (i) it may comprise 10 cells or fewer. In order to work out the copy number of the nucleic acid, it is necessary to know the exact number of cells. In one embodiment, a single cell is lysed to provide the lysate of step (i). An advantage of using a single cell is that it avoids any inaccuracy associated with obtaining the cell number. Page: 3 Another advantage is that it determines copy-number unambiguously for that cell; with two or more cells, the total number of copies may be known, but there is no guarantee that all the cells have the same copy-number.

In a second aspect, the present invention provides a method for counting the absolute copy number of a chromatid in a cell by counting the copy number of one or more nucleic acid marker(s) unique to the chromatid using a method according to any preceding claim.

The copy number of a plurality of nucleic acid markers from the chromatid may be determined in order to analyse multiple loci on each chromatid. The plurality of nucleic acid markers may comprise one or more pairs or multiples of markers which occur in close proximity on the chromatid. This helps to monitor for PCR failure due to “allele dropout” (see below).

It is theoretically possible for sister chromatids to be apportioned to the same aliquot (co-segregate) which may lead to an underestimation of the chromatid number. Such errors can be overcome by analysing a plurality of markers for a given chromosome. Since the chromosomes break upon isolation, the markers segregate independently, so it is unlikely that co-segregation of one marker will occur at the same time as co-segregation of another marker, provided that the markers are far apart on the chromosome. In connection with this embodiment, the plurality of nucleic acid markers may comprise markers which occur far apart on the chromatid.

Where the method comprises analysis of a plurality of markers, the highest number indicated gives the absolute copy number of the nucleic acid in the cell. Markers which give a number lower than this maximum may represent an underestimate due to co-segregation and/or allele drop-out. These lower numbers can therefore be ignored.

The most frequent aneuploidies in humans are trisomy 21, 18 and 13. Hence, the method of the invention may involve counting the copy number of chromatids from one or more chromosomes 21, 18 or 13.

The method may count the absolute copy number of a plurality of chromatids in the cell, for example it may count the chromatids from at least 3 chromosomes such as chromosomes 21, 18 and/or 13.

In a third aspect, the present invention provides a method for investigating the ploidy status of a cell, by counting the absolute copy number of chromatids for each chromosome in the cell by a method according to the second aspect of the invention.

The “cell” may be a cell structure such as a polar body.

The cell may be derived from a cleavage stage embryo.

The cell may be a trophectoderm cell of a blastocyst.

The cell may be a fetal cell, for example from an amniotic fluid or a chorionic villus sample.

The cell may be in telophase.

In a fourth aspect, the present invention provides a method for counting the copy number of a chromatid in an oocyte, which comprises the step of counting the copy number of the chromatid in the oocyte-associated cell body by a method according to the second aspect of the invention and directly deducing the copy number of the chromatid in the oocyte.

In a fifth aspect, the present invention provides a method for investigating the ploidy status of an oocyte by investigating the ploidy status of the oocyte-associated polar body by a method according to the third aspect of the invention and directly deducing the ploidy status of the oocyte.

The oocyte may be from a human subject of 35 years or older. The oocyte may be from a human subject (of any age) who has fertility problems or has or carries an inheritable disease. The oocyte may be from a human subject undergoing IVF treatment.

In a sixth aspect, the present invention provides a method for in vitro fertilisation of an oocyte, which comprises the step of selecting an oocyte determined to be euploid by a method according to the fifth aspect of the invention.

The ploidy status of both polar body I and polar body II may be investigated.

In a seventh aspect the present invention provides a method for investigating the ploidy status of an embryo by investigating the ploidy status of an embryo-derived cell(s) by a method according to the fifth aspect of the invention.

In an eighth aspect, the present invention provides a primer set for use in a method according to the second aspect of the invention, which comprises a plurality of primers capable of amplifying a plurality of nucleic acid markers from a chromatid.

The set may comprise primers capable of amplifying one or more nucleic acid markers from a chromatid from each chromosome in the cell.

The set may comprise primers to amplify at least four nucleic acid markers per chromatid.

The set may comprise one or more primer(s) capable of amplifying or detecting a disease-specific gene, allele or mutation.

The set may comprise primers capable of amplifying one or more pairs or multiples of nucleic acid markers which occur in close proximity on the or each chromatid and/or primers capable of amplifying one or more pairs or multiples of nucleic acid markers which occur far apart on the or each chromatid.

As the method of the invention counts chromatids directly, this system is the only technique to date that allows detection of all kinds of malsegregation of chromosomal material for all chromosomes. It is thus the only technique which provides full and accurate information on the ploidy status of a cell.

Other major advantages of the method include the following:
(i) unlike other DNA counting techniques the method of the present invention does not require whole genome amplification or any hybridisation step. This obviates any problems that might arise from incomplete genomic coverage, region specific genome amplification, incomplete suppression of repeat sequences within the probe and removes any risk of cross-hybridisation, as can occur in short oligo arrays. There is also no need of DNA labelling with fluorescent dyes and metaphase chromosomes or BAC clones for hybridisation;
(ii) as the method is essentially digital (counting molecules), interpretation of the results is simplified, in contrast with, for example micro-array approaches, which can require complex algorithms for interpretation;
(iii) unlike methods such as FISH, the method of the invention is suitable for automation and high throughput while still being easily applicable for manual operations such as gel electrophoresis. Therefore the method of the invention has no mandatory requirement for machinery, such as arrayers.
(iv) with the method of the invention, a highly desirable time frame can be achieved. Array based methods generally need at least 48 hours to obtain a result, making embryo freezing and implantation at a consecutive cycle necessary. With the method of the invention, on the other hand, a result for all chromosomes can be obtained within 24 hours. Thus if the method of the present invention is used to investigate the ploidy status of an embryo, this obviates the need for freezing and implantation in a subsequent cycle; and
(v) when the method of the present invention is used on fetal cells, a significant reduction of time by which the diagnosis can be delivered can be achieved, compared to the time needed before conventional cytogenetic karyotyping, as there is need only of a few dividing cells (1 week instead of 2 weeks).

### DESCRIPTION OF THE FIGURES

FIG. 1. Meiosis I is initiated during fetal development.

After homologous chromosome synapsis and initiation of recombination, meiosis arrests in the first meiotic prophase and is only resumed at ovulation. After completion of meiosis I the oocyte undergoes meiosis II and arrests in metaphase. If no fertilisation takes place the oocyte is degraded; if fertilised meiosis II is completed.

FIG. 2. Results of chromosome segregation and malsegregation in meiosis I.

A normal meiotic division results in the segregation of two homologous chromosomes with 2 chromatids each (euploidy). In the case of chromosome non-disjunction both homologous chromosomes segregate to the same pole leading to either quatrosomy or nullisomy in the oocyte. The other frequent mechanism is early sister-chromatid separation leading to either trisomy or monosomy in the oocyte.

FIG. 3. Chromatid counting through single cell MCC.

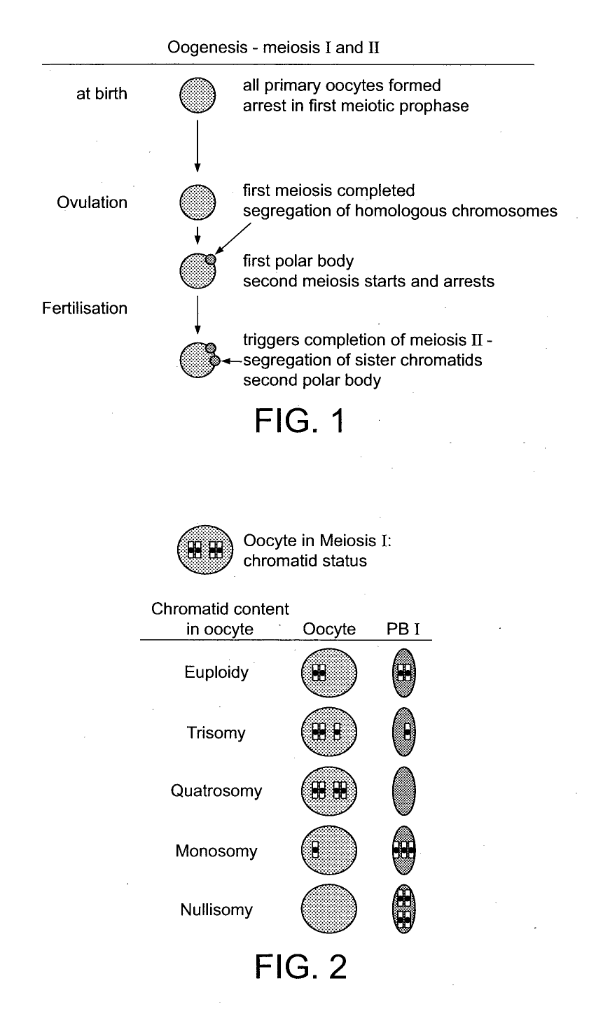

PB I is lysed and the cell lysate is dispensed over 8 PCR reaction wells (aliquots), leading to single DNA molecules at limiting dilution with 0.25 genomes per PCR well in the case of euploidy. After 2 rounds of specific PCR amplifications the number of chromatids per chromosome is analysed by simply counting the numbers of positive PCR reactions representing target sequences on all chromosomes. In this example, the DNA content is divided into only 8 aliquots, raising the possibility that two chromatids may occasionally co-segregate (ie, be apportioned to the same aliquot) and be mis-counted as one. Such errors can be overcome either by dividing the sample into more aliquots (reducing the chances of co-segregation), or by analysing multiple markers scattered along each chromosome (since the chromosomes break upon isolation, so that the markers segregate independently and hence co-segregation of two copies of one marker will not occur at the same time as co-segregation of two copies of another marker).

FIG. 4. Analysis of a polar body I with 4 markers per chromosome.

PB I is expected to contain 2 copies for all chromosomes and was diluted into 8 aliquots which equals an average DNA content of 0.25 genomes per aliquot. The 4 markers analysed per chromosome were not linked but rather in distances of several megabases. As the primer panel used for this experiment had not been optimised there are several markers which did not work at all or were not robust in consecutive analyses; they are indicated by omission of the primer name. In cases of a missing result in the presence of the proper primer name allele drop out has occurred which is the case for markers 7, 19, 28, 30, 37, 38, 39, 45, 57, 69, 76 and 82. Markers 93-96 cannot be judged as no Y chromosome is present in polar bodies.

FIG. 5. Analysis of a fibroblast at telophase.

The cell was expected to contain 4 copies for all autosomes and 2 copies for chromosomes X and Y and was diluted into 16 aliquots which equals 0.25 genomes per aliquot for the autosomes and 0.125 genomes for the sex chromosomes. The markers used here were linked with 24 markers per chromosome, the chromosomes being chromosomes 10, 21, X and Y. The furthest column to the right gives the counts of positive PCRs per marker, green fields being in accordance with the expected numbers of positives. Again this marker panel was not optimised but demonstrates that the presence of chromatids can be verified. The shift of counts from 4 to 2 nicely reflects the reduction of chromatids from 4 to 2 as from autosomes to sex chromosomes. Moreover linkage can be observed along the markers showing that the DNA strands are intact over several kilobases. Use of a robust primer set with closely linked markers allows one to estimate how much allele drop out occurs, by observing linkage.

FIG. 6. Single cell MCC of polar body I and II with sensitivity at the chromatid level.

(a). Examples of euploid chromosomes.
(b). Example of euploid chromosome 14 and aneuploid chromosome 15 due to a meiosis II error.
(c). Meiosis I error resulting in a trisomy of the zygote.
(d). Repair of a meiosis I error with resulting euploidy.

FIG. 7. Increase of result robustness through remote and clustered markers.

In this scheme a PB1 has been analysed with markers on selected chromosomes. Markers are composed of 2×4 clustered markers per chromosome thus analysing 2 independent regions per chromosome at a redundacy of 4. Blue boxes indicate the PCR aliquot with a positive PCR, numbers within the boxes are the melting temperatures of the PCR products which are specific for each marker. With our lysis protocol DNA molecules have a length of several kb thus resulting in good linkage patterns. PCR products marked orange are judged as false positives as DNA from external contamination is more fragmented therefore giving the random odd additional signal. In this analysis there is only one marker with a false too low result—the forth marker on Xp. The linkage pattern clearly indicates that it has to be ADO as all other markers give 2 signals in identical PCR aliquots.

FIG. 8. Strategy to ensure results for all chromosomes.

A combination of independent and linked markers distributed along all chromosomes should provide sufficient redundancy to compensate for signal loss due to DNA fragmentation, ADO and cosegregation. Each block of markers (brown and yellow) represents linked markers with distances of 500-1000 bp interrogating 6 independent regions with 2 (brown) and 4 (yellow) markers per region, each marker confirming the result of the other markers per region.

### DETAILED DESCRIPTION

### Copy Number

In a first aspect, the present invention provides a method for counting the copy number of a nucleic acid sequence in a cell.

The copy number is the number of copies of the nucleic acid sequence in the genome of the cell.

The method comprises the steps of
(i) dividing a lysate of the cell into a plurality of aliquots;
(ii) providing conditions suitable for the amplification of the nucleic acid sequence in each aliquot;
(iii) counting the number of aliquots in which the nucleic acid was amplified in step (ii) thus the copy number of the nucleic acid sequence in the cell.

The number of aliquots which test positive give an absolute number for the copy number of nucleic acids in the cell. For example, if a single cell is lysed and the lysate split into multiple aliquots, two of which test positive by polymerase chain reaction (PCR-see below), it can be directly deduced that the cell contained two copies of the nucleic acid. For a single cell, the number of positive wells equates with the copy number of the nucleic acid, assuming there is no co-segregation, which is explained in more detail below.

It is possible to perform the method using more than one cell, as long as the exact number of cells in the sample is known or can be derived. For example, if two cells are lysed and the lysate split into multiple aliquots, four of which test positive by PCR, it can be directly deduced that the cells each contain two copies of the nucleic acid. The copy number of the nucleic acid per cell may be directly calculated by dividing the number of aliquots which test positive with the number of cells in the sample.

WO 2007/129000 describes a method of measuring the copy number frequency of one or more nucleic acids in a sample by comparing the frequency with which PCR amplification occurs of a) a test marker and b) a reference marker at limiting dilution.

In the method of WO 2007/129000 the objective is to discover the average number of copies of a given marker in a population of cells (typically at least ten cells). Using this method, one arrives at an estimate of mean copy-number by statistical methods. The amount of DNA per aliquot is chosen such that a large proportion (typically 50%) of aliquots are positive for the marker sequence leading to a high rate of co-segregation, and the results are deconvoluted statistically. In the method of present invention, on the other hand, the amount of DNA per aliquot is ideally small enough that co-segregation is rare; and rather than derive a statistical estimate of copy-number, the method provides an exact copy-number for a given nucleic acid in a cell.

The method of WO 2007/129000 uses processed genomic DNA, produced by a method involving cleaning steps. By contrast, in the method of the present invention, the total cell content plus lysis buffer is put into the PCR reaction as any cleaning step would be likely to cause a loss of material, i.e. loss of DNA.

### Providing and Correcting for Under-Estimation

In the method of the present invention it is possible that two copies of a given target sequence (“marker”) may happen to fall into the same aliquot as the DNA is divided (ie, they may “co-segregate”). Since PCR detects only the presence or absence of the marker in an aliquot, such instances lead to an under-counting of the copies of that marker. Such co-segregation, and hence under-counting, is statistically simple to predict and to take into account.

Errors arising from co-segregation can be reduced by splitting the DNA into more aliquots, so that co-segregation becomes less likely.

The cell lysate may be split into at least 5, 10, 15, 20 or more aliquots.

Each aliquot may have an average of 0.25 genomes per aliquot or less, for example 0.20, 0.15 or 0.1 genomes per aliquot or less.

Alternatively, or in addition, errors arising from co-segregation can be reduced by analysing multiple markers within the same nucleic acid sequence.

For example, where the method of the present invention is used for chromatid counting, chromatids break upon extraction, so that if multiple markers are used, they behave independently especially if they are far enough apart on the chromatid. Thus, whilst two copies of one chromatid marker may co-segregate and lead to an underestimate of chromatid number in that cell, two copies of another marker on the same chromosome may not. Where multiple markers are used in this way, the true chromatid number of the cell is the highest number indicated by any of the markers.

Errors may also arise due to PCR failure (“allele dropout”). This can be addressed by selecting markers known to amplify efficiently, by using multiple markers on each chromosome, and/or by using pairs of markers which are nearly adjacent on the chromosome. In this last case, one would expect both members of a pair to co-segregate (since the DNA is unlikely to break in the very small interval between them); failure of co-segregation of such paired markers would be indicative of PCR failure. The same approach can be extended to use triplets (or more) of markers in the same way.

It is difficult to rule out undesired co-segregation and allele dropout completely. However, they can be kept within manageable limits, and their frequency can be either predicted (co-segregation) or monitored (allele dropout). By analysing multiple loci on each chromosome, one can obtain a nucleic acid copy number and a measure of confidence in that number.

FIGS. 7 and 8 show strategies for maximising robustness of the method.

### Nucleic Acid

The term “nucleic acid” as used herein refers to a deoxyribonucleotide or ribonucleotide in either single or double-stranded form.

The nucleic acid may be genomic DNA.

The nucleic acid may be part of a chromatid or a chromosome.

A chromatid is one of the two identical copies of DNA making up a chromosome, which are joined at their centromeres. When the centromeres separate (during anaphase of mitosis and anaphase 2 of meiosis), the two strands are called sister chromatids.

The chromatid may be from a chromosome which is commonly associated with aneuploidy, such as chromosomes 21, 18 and 13.

In addition to counting chromatids, the method of the invention may be used for many other applications which involve a copy number change, for example nonreciprocal translocations, deletions or trinucleotide repeat disorders. It is even possible to detect reciprocal translocations and inversions by using linked markers spanning the breakpoints.

### Cell

The cell under investigation using the method of the present invention may be a haploid or diploid cell.

The cell may be derivable from a cell sample such as a blood, plasma, serum, saliva, urine, tears, tissue, lymph, or tumour sample.

The cell may be a gamete such as an oocyte or a sperm cell.

The “cell” may be a cell structure such as a polar body.

Asymmetrical cell division (cytokinesis) leads to the production of polar bodies during oogenesis.

There may be one or two polar bodies in the oocyte. The first polar body is one of the two products after completion of meiosis I and may be considered haploid, with 23 duplicated chromosomes in humans (one of each pair of homologous chromosomes). The second polar body is also haploid, with 23 unduplicated chromosomes. Both are relatively small and contain little cytoplasm.

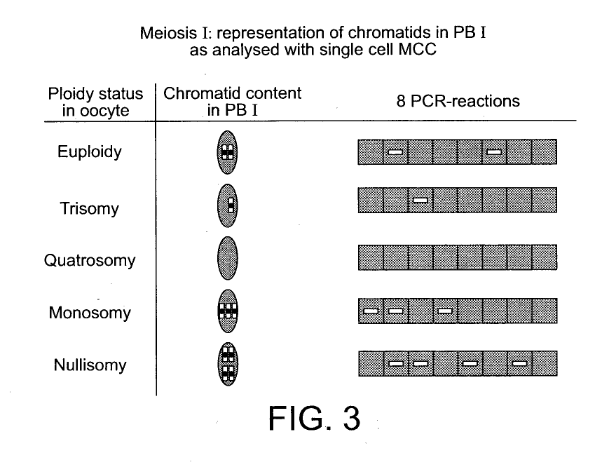

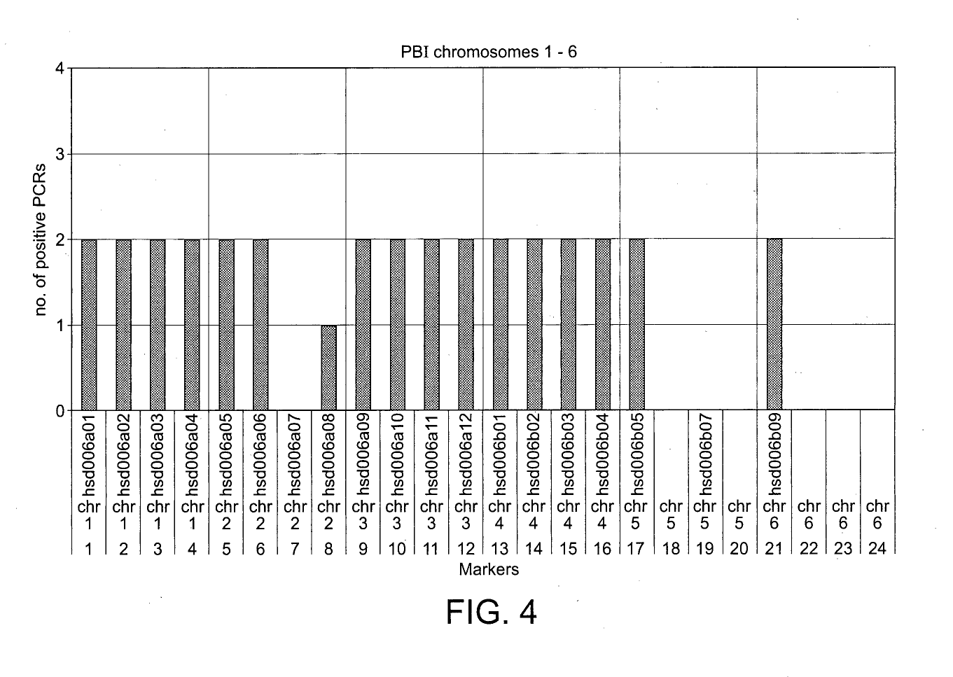

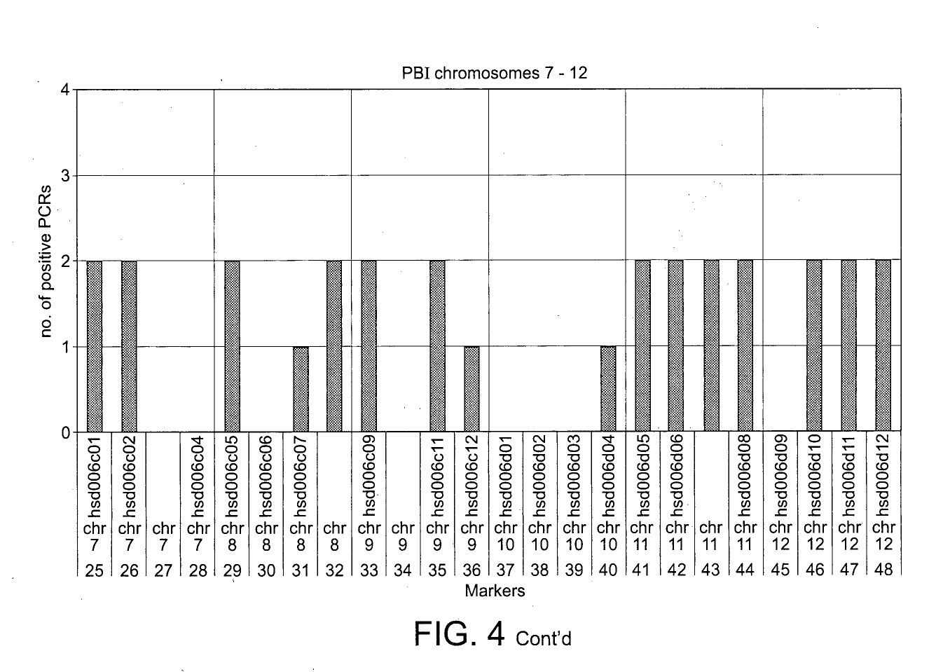

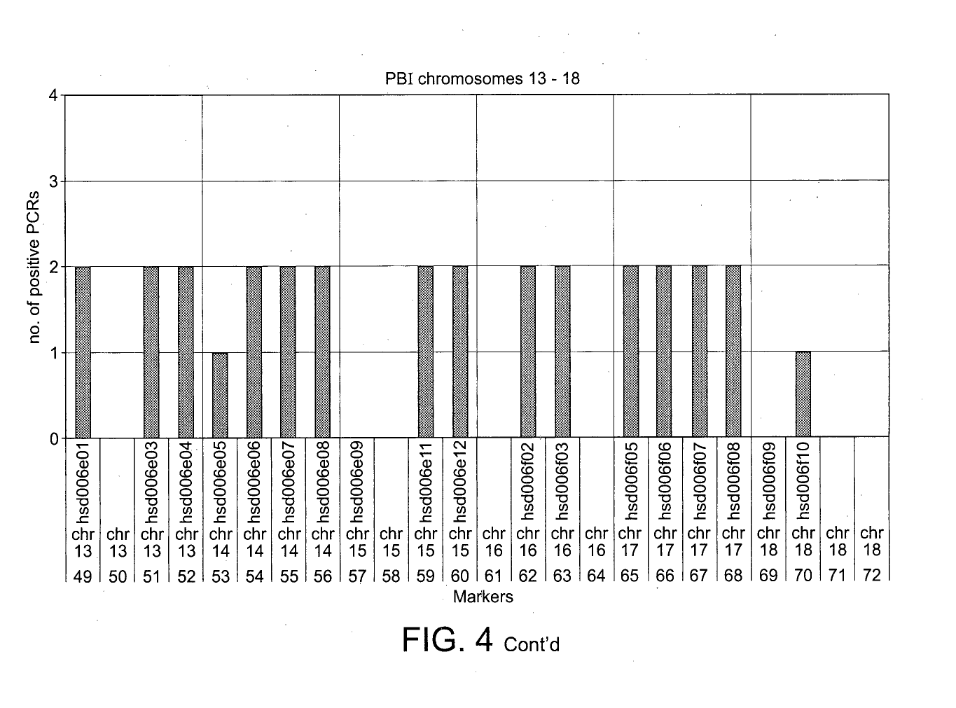

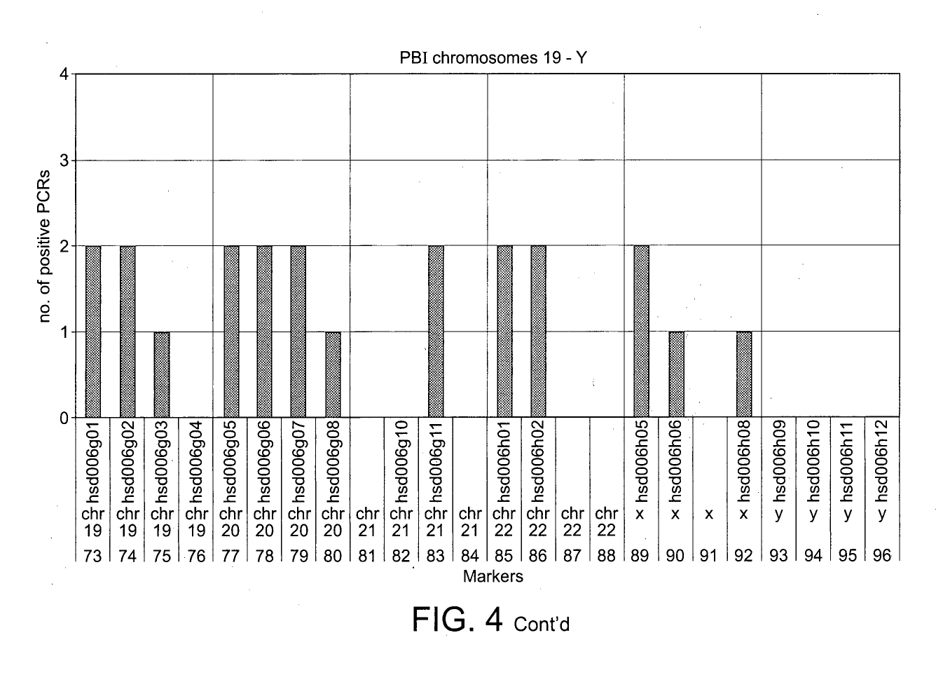

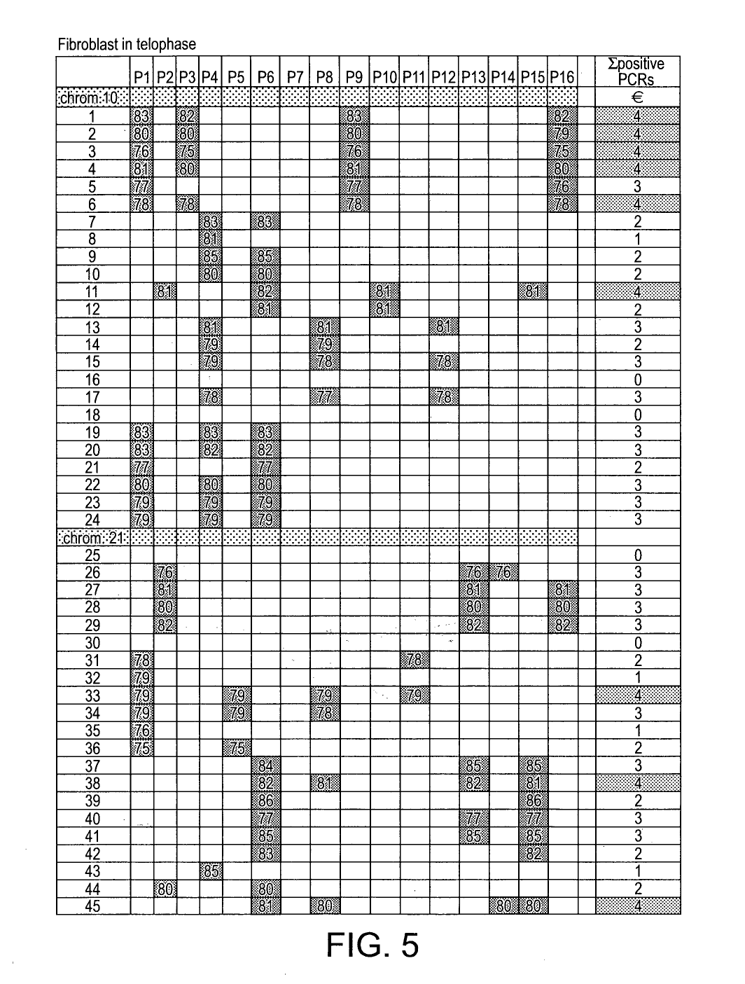

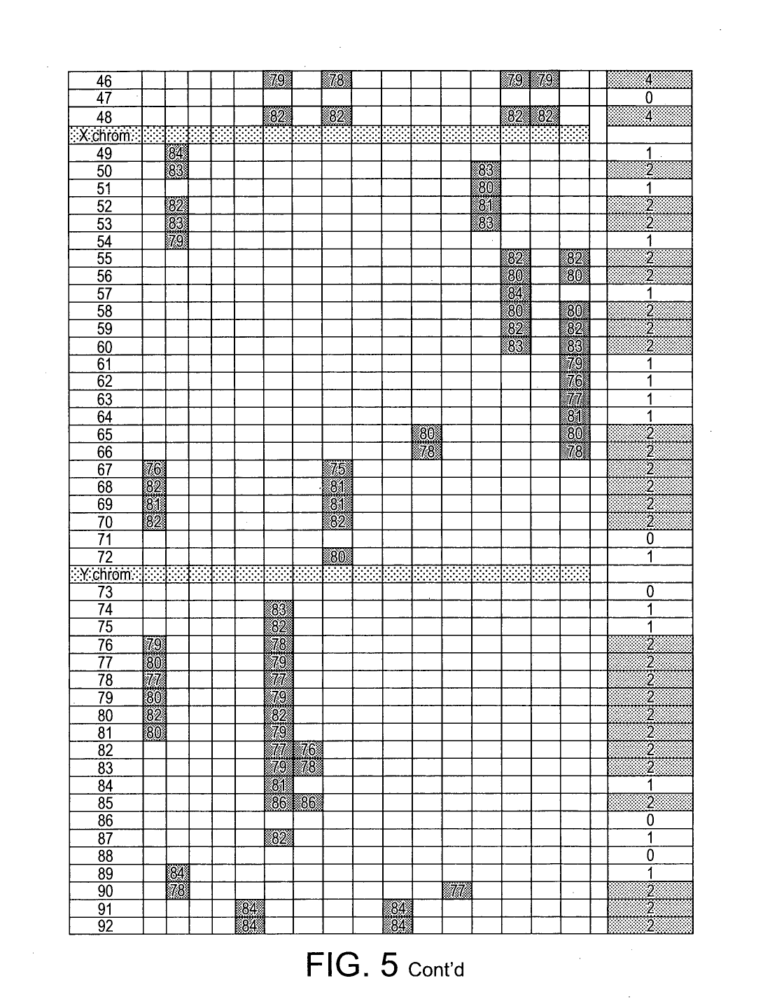

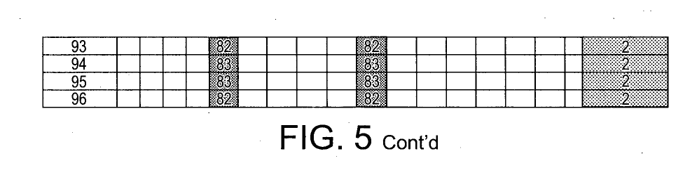

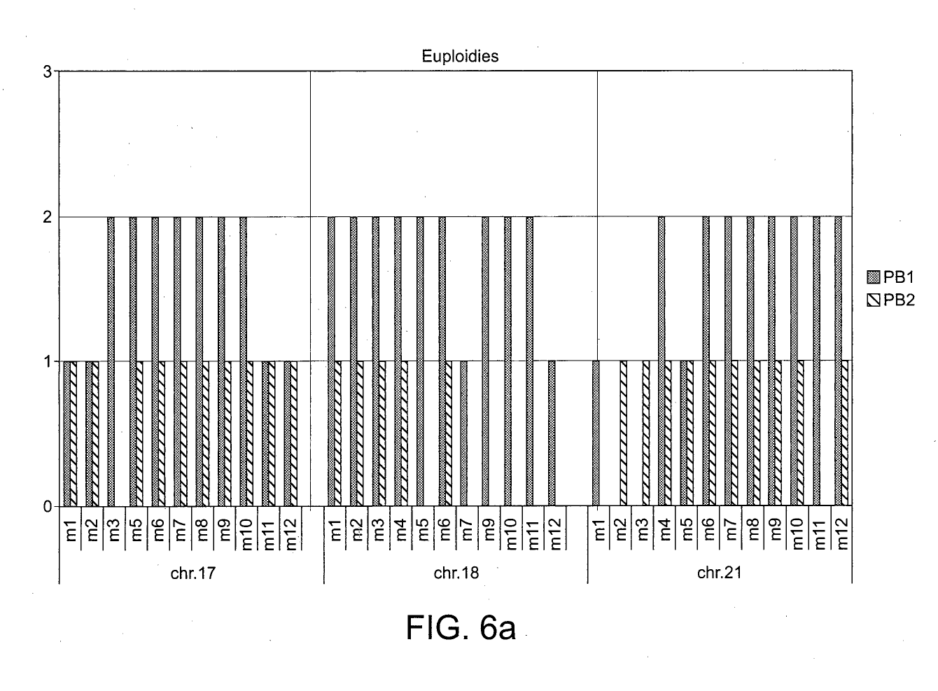

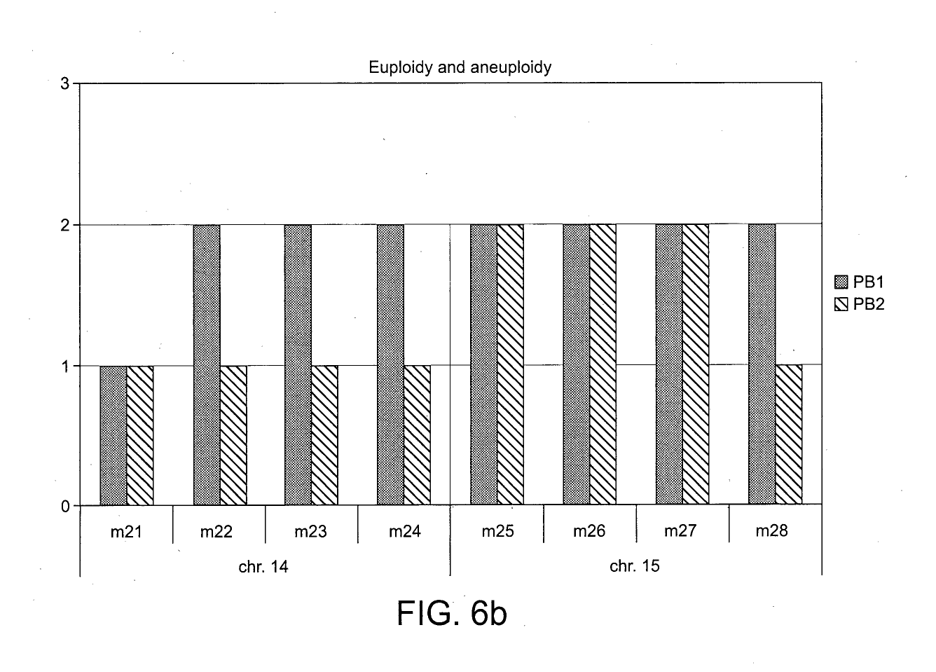

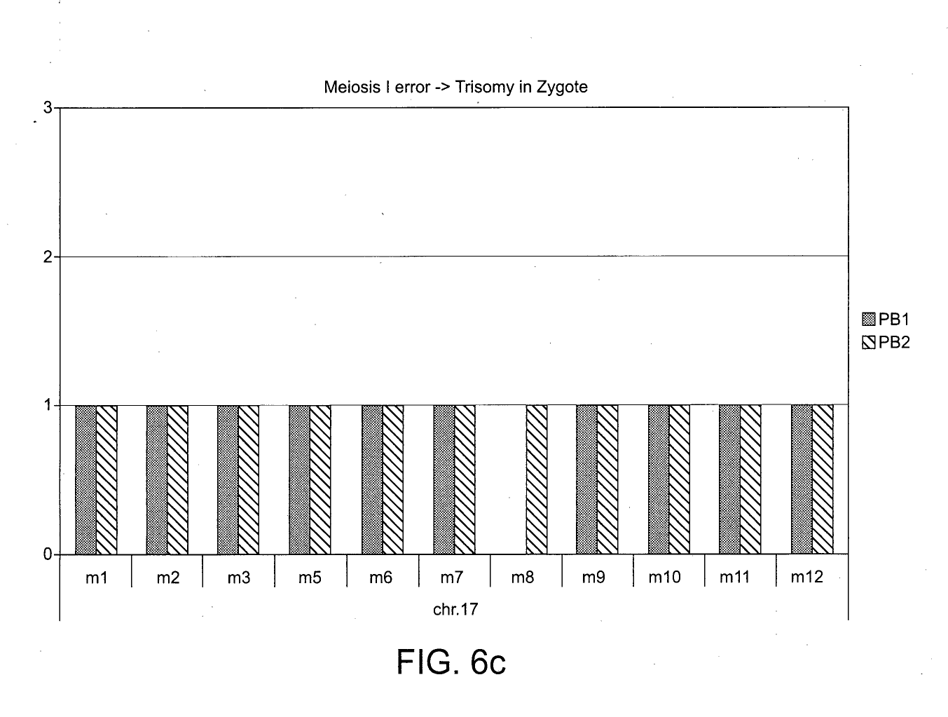

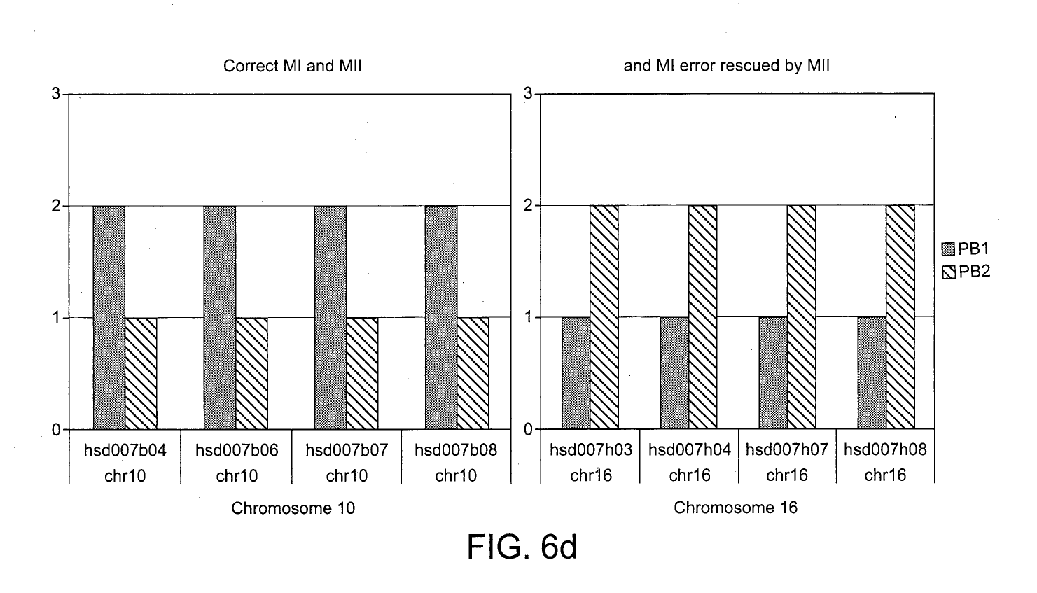

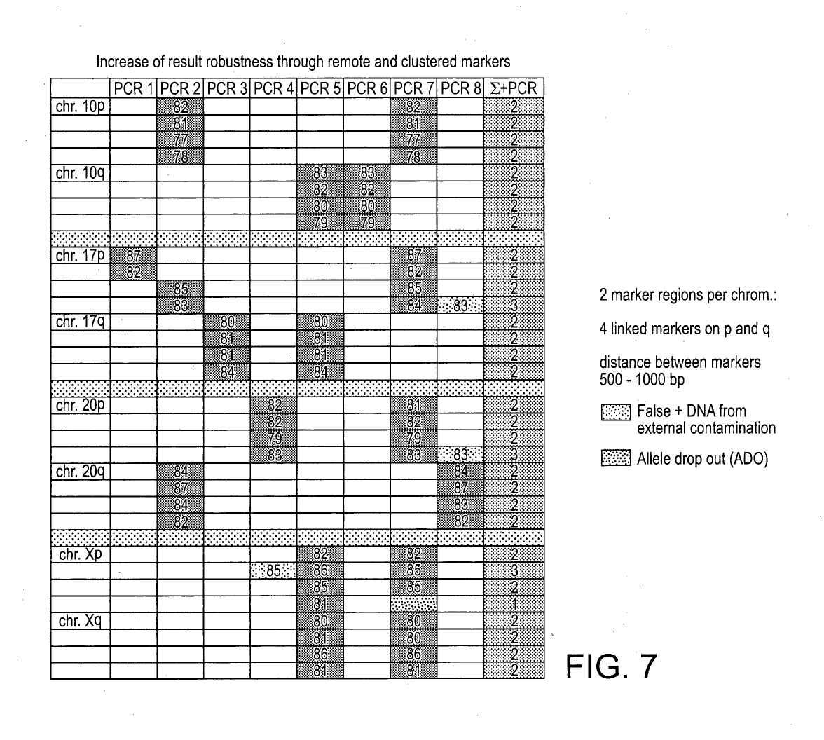

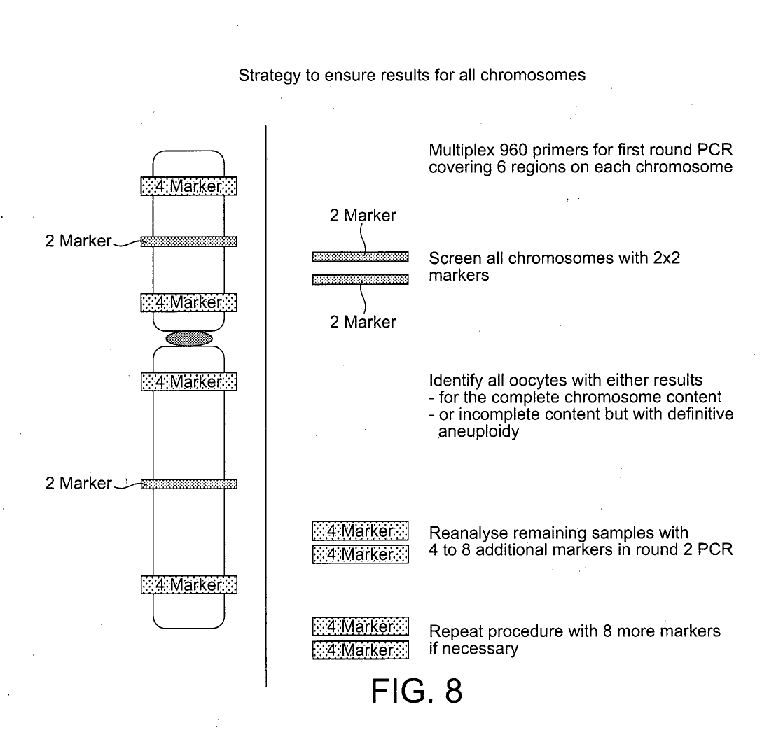

Polar bodies are the by-products of the egg's division during meiosis. As an egg matures, it goes through a two-step division process, dividing once at the time when ovulation would occur and again at the time of fertilization. The two haploid polar bodies are the by-products of this division, and are essentially discarded by the egg. By analyzing the polar bodies, it is possible to infer the genetic status of the egg, as shown in FIG. 3 and FIG. 6a-d.

The cell may be derivable from a pre-implantation embryo. For example, the cell may be derivable from a cleavage stage embryo or from a blastocyst. The cell may be a trophectoderm cell from a blastocyst.

The cell may be derivable from a post-implantation embryo. For example, the cell may be an embryonic cell derivable from an ongoing pregnancy, such as a cell from an amniotic fluid or chorionic villus sample.

The oocyte or embryo may be from or for a female subject who has one or more of the following:
(i) advanced maternal age, for example at least 35, 37 or 40 years;
(ii) a past history of repeated implantation failure; and/or
(iii) a past history of repeated miscarriage.

The female subject may be about to undergo IVF treatment or may have an ongoing pregnancy as a result of IVF treatment. The IVF treatment may involve single embryo transfer.

The cell may be at telophase. Telophase is the final stage of both mitosis and meiosis, when a new nuclear envelope forms around each set of chromosomes and both sets of chromosomes unfold back into chromatin. The distinguished shape of cells in telophase allows for the selection of single cells at a defined chromosome status, i.e. all chromosome pairs in metaphase with 2 chromatids each, giving 4 copies.

### Cell Sample

As mentioned above, it is possible to perform the method of the invention with a plurality of cells, as long as the number of cells is known or can be derived.

The cell sample may have 10 or fewer, 5 or fewer, 3 or 2 cells.

The number of cells in the cell sample may be counted or derived by methods known in the art. For example FACS sorting may be used, or cell may be collected, for example with a micropipette, and directly counted under a microscope using visual control.

### Single Gene Defects

The method of the invention may also be used to investigate single gene defects and for mutation screening in the cell. The method of the invention is highly flexible when it comes to the composition of amplification primers, and so primers may be included which amplify disease specific genes or alleles to allow assessment of disease risk. A non-exhaustive list of such single gene disorders is given in Table I.

TABLE 1

| Single gene disorder | Gene |
|---|---|
| Adrenoleukodystrophy (ALD) | ABCD1 |
| Charcot Marie Tooth type 1A (CMT1A) | PMP22 |
| Cystic Fibrosis (CF) | CFTR |
| Congenital adrenal hyperplasia (CAH) | CYP21A2 |
| Crigler-Najjar syndrome | UGT1A1 |
| Deafness, autosomal recessive | CX26 |
| Duchenne-Becker muscular dystrophy (DMD/DMB) | DMD |
| Duncan disease - X-linked lymphoproliferative syndrome | SH2D1A |
| (XLPD) | |
| Ectrodactyly ectodermal dysplasia and cleft lip/ | p63 |
| palate syndrome (EEC) | |
| Epidermolysis bullosa dystrophica/pruriginosa | COL7A1 |
| Exostoses multiple type I (EXT1) | EXT1 |
| Exostoses multiple type II (EXT2) | EXT2 |
| Facioscapulohumeral muscular dystrophy | FRG1 |
| Factor VII deficiency | F7 |
| Familial Mediterranean Fever (FMF) | MEFV |
| Fanconi anemia A | FANCA |
| Fanconi anemia G | FANCG |
| Fragile-X | FRAXA |
| Gangliosidosis (GM1) | GLB1 |
| Gaucher disease (GD) | GBA |
| Glucose-6-phosphate dehydrogenase deficiency | G6PD |
| Haemophilia A | F8 |
| Haemophilia B | F9 |
| HLA typing | HLA |
| Lesch-Nyhan syndrome | HPRT |
| Limb-girdle muscular dystrophy type 2C (LGMD2C) | SGCG |
| Marfan syndrome | FBN1 |
| Myotonic dystrophy (DM) | DMPK |
| Neurofibromatosis 1 | NF1 |
| Neurofibromatosis 2 | NF2 |
| Phenylketonuria | PAH |
| Polycystic kidney disease type 1 (PKD1) | PKD1 |
| Polycystic kidney disease type 2 (PKD2) | PKD2 |
| Sickle cell anemia | HBB |
| Spastic paraplegia type 3 | SPG3A |
| Spinal Muscular Atrophy (SMA) | SMN |
| Spinocerebellar ataxia 3 (SCA3) | ATXN3 |
| Spinocerebellar ataxia 7 (SCA7) | ATXN7 |
| Stargardt disease | ABCA4 |
| Tay Sachs (TSD) | HEXA |
| Thalassemia-α mental retardation syndrome | ATRX |
| Thalassemia-β | HBB |
| Tuberosclerosis 1 | TSC1 |
| Tuberosclerosis 2 | TSC2 |
| Von Hippel-Lindau syndrome | VHL |
| Wiskott-Aldrich Sindrome (WAS) | WAS |

Disease risk of the maternal genomic content may be investigated in the case of PB diagnosis, whereas that of both maternal and paternal genomic content may be investigated if embryo or trophectoderm biopsies are performed.

### Amplification

As used herein, “amplification” refers to any process for multiplying strands of nucleic acid, such as genomic DNA, in vitro.

Amplification techniques include thermal cycling amplification methods, such as ligase chain reaction; and isothermal amplification methods, such as Strand Displacement Amplification (SDA), Q-beta replicase, nucleic acid-based Sequence Amplification (NASBA); and Self-Sustained Sequence Replication.

The amplification method may be polymerase chain reaction (PCR). PCR involves using paired sets of oligonucleotides of predetermined sequence that hybridise to opposite strands of DNA and define the limits of the sequence to be amplified. The oligonucleotides prime multiple sequential rounds of DNA synthesis catalysed by a thermostable DNA polymerase. Each round of synthesis is typically separated by a melting and re-annealing step, allowing a given DNA sequence to be amplified several hundred-fold in less than an hour.

The amplification step may be automated, making the method suitable for use in high-throughput screening techniques.

### Markers

The nucleic acid sequence whose copy number is being determined may be a “marker” for a longer nucleic acid sequence. For example, it may be a marker for a section of genomic DNA, a chromatid or a chromosome.

For chromatid counting, the method may be used to count the number of a plurality of markers for each chromosome. This provides an internal cross-reference for the correct copy number for the chromatid. For the reasons explained above (co-segregation and allele drop-out), a given marker may produce an underestimation for the copy number. If a plurality of markers is used, this can be checked. The marker(s) giving the highest copy number (assuming there is no PCR contamination) can be assumed to give the correct number.

To check for and take steps to avoid errors due to co-segregation, markers may be chosen which are spaced far apart on the chromatid. For example, the markers may be separated by at least 500 kb, at least 1 Mb, at least 3 Mb or at least 5 Mb.

To check for and take steps to avoid errors due to allele drop out, markers may be chosen which amplify nucleic acids in close proximity on the chromatid. For example, the nucleic acids may be spaced by less than 2 kb, for example between 50 and 500 bp.

The marker nucleic acid sequence may be any length that is amplifiable by the chosen method. A disadvantage of using very long marker sequences is that the likelihood of allele drop out is increased. Typically marker sequences are chosen which are 75-130 bp in length.

### Ploidy Status

Ploidy corresponds to the number of chromosomes in a cell. In humans, somatic cells are diploid, containing two complete sets of chromosomes, one set derived from each parent; and gametes are haploid.

The number of chromosomes in a single non-homologous set is called the monoploid number (x). The haploid number (n) is the number of chromosomes in a gamete of an individual. Both of these numbers apply to every cell of a given organism. For humans, x=n=23; a diploid human cell contains 46 chromosomes: 2 complete haploid sets, or 23 homologous chromosome pairs (for a female; a male has 22 homologous chromosome pairs, one X and one Y chromosome).

Euploidy is the state of a cell or organism having an integral multiple of the monoploid number. For example, a human cell has 46 chromosomes, which is an integer multiple of the monoploid number, 23. Aneuploidy is the state of not having euploidy. In humans, examples include having a single extra chromosome (such as Down syndrome), or missing a chromosome (such as Turner syndrome).

During oocyte maturation, normal division in meiosis I results in the segregation of two homologous chromosomes, one remaining in the oocyte and one extruded to the polar body, so that both the polar body and the oocyte have two chromatids each (euploidy). If an error occurs, the sharing of chromatids between oocyte and polar body may be unequal, leading to aneuploidy in both the polar body and the oocyte (see FIG. 2).

Using the method of the invention, it is possible to investigate the ploidy status of a cell or polar body for one or more chromosomes. The method may be used for all 22 chromosomes together with X and (if appropriate) Y, producing a complete picture of the ploidy status of the cell.

### PRIMER SET

The fifth aspect of the present invention relates to a primer set which comprises primers capable of amplifying a nucleic acid in accordance with step (ii) of the method of the first aspect of the invention.

The term “primer” is used herein interchangeably with “oligonucleotide” to mean a short length of nucleic acid which hybridises specifically to a target sequence enabling the nucleic acid sequence whose copy number is to be determined (i.e. the marker sequence) to be amplified.

The primers may be capable of hybridising at flanking regions of the nucleic acid marker sequence. The primers are chosen to have at least substantial complementarity with the different strands of the nucleic acid being amplified.

The primer must have sufficient length so that it is capable of priming the synthesis of extension products. The length and composition of the primer depends on many factors including, for example, the temperature at which the annealing reaction is conducted, concentration of primer and the particular nucleic acid composition of the primer. Typically the primer has 15-30 nucleotides, such as 18-20 bp.

The term “hybridise specifically” refers to hybridisation of the primer to the target sequence under stringent conditions, that is conditions under which a primer will hybridise preferentially to its target sequence and to a lesser extent to, or not at all to, other sequences.

The primer set may comprise two primers for each marker sequence: one “forward” and one “reverse” primer. Alternatively the primer set may comprise three primers in a hemi-nested configuration.

The set may comprise primers capable of amplifying one or more nucleic acid markers from a chromatid. The set may comprise primers capable of amplifying a plurality of nucleic acid markers from a chromatid. For example, the set may comprise primers capable of amplifying at least 4, 6, 8, 10, 15, 20, 25 or more markers for the chromatid or for each chromatid.

The set may comprise primers capable of amplifying one or more nucleic acid markers from a plurality of chromatids in the cell. For example, the set may comprise primers capable of amplifying markers from at least 3, 5, 8, 12 or 15 chromosomes. The set may comprise primers capable of amplifying markers from each chromosome in the cell.

The set may comprise one or more primer(s) capable of amplifying or detecting a disease-specific gene, allele or mutation.

The set may comprise primers capable of amplifying one or more pairs or multiples of nucleic acid markers which occur in close proximity on the or each chromatid and/or primers capable of amplifying one or more pairs or multiples of nucleic acid markers which occur far apart on the or each chromatid.

The primer set may be provided as part of a PCR kit, which may also contain deoxynucleotide triphosphates and/or Taq polymerase.

The kit may also comprise one or more container(s) and instructions for use.

As the method is highly suited for automated methods, such as high-throughput screening, the primer set may be provided as part of a multi-well plate, such as a 96-well plate, each well being ready to receive and aliquot of lysate.

The invention will now be further described by way of Examples, which are meant to serve to assist one of ordinary skill in the art in carrying out the invention and are not intended in any way to limit the scope of the invention.

### EXAMPLES

### Example 1

### Investigation of the Ploidy Status of Polar Body I

The ploidy status of an oocyte was ascertained by investigating the ploidy status of polar body I (PBI) using the chromatid counting method of the invention with four markers per chromosome.

The polar body was lysed and dispensed into 8 aliquots. PBI is expected to contain 2 copies for all chromosomes, so each aliquot comprises an average DNA content of 0.25 genomes per aliquot.

As shown in FIG. 4, PBI was confirmed to be haploid with 2n for the following chromosomes 1 to 9, 11 to 17 and 19-22 and X. Chromosomes 10 and 18 each gave only one positive PCR and are judged as technical failure.

### Example 2

### Investigation of the Ploidy Status of a Fibroblast at Telophase

A diploid fibroblast at telophase is expected to contain 4 copies of each autosome and 4 copies of X in females; or 2 copies of X and two copies of Y in males.

A fibroblast at telophase was selected due to its distinguished shape, lysed and divided into 16 aliquots. As for example 1, this gives an average of 0.25 genomes/aliquot for the autosomes and X in the female fibroblast and 0.125 genomes/aliquot for X and Y in the male fibrobast. Linked markes are used for four chromosomes: namely chromosomes 10, 21, X and Y.

As shown in FIG. 5, it was confirmed that the fibroblast in telophase contained 4 copies of chromatids from chromosomes 10 and 21 and two copies of each of the chromatids from the X and Y chromosomes.

This is the first time that the chromosome content of a single cell has been resolved at the chromatid level allowing one to detect directly not only chromosome disjunctions for all chromosomes but also early sister-chromatid separation.

### Example 3

### Single cell MCC of solar body I and II with sensitivity at the chromatid level

### (a) Examples of Euploid Chromosomes.

After correct meiosis I and II polar body I (PB1, PB2) contains 2 copies for all chromosomes while PB2 contains I copy. This is shown for chromosomes 17, 18 and 21 with a set of 12 markers per chromosomes with 2 linked groups of 6 markers (FIG. 6 (a); marker 4 chrom. 17 and marker 8 chrom. 18 did not work and were removed). In most cases 2 chromatids, i.e. 2 positive PCRs are shown in PB1 (red) and 1 chromatid in PB2 (blue). Discrimination between 1 and 2 copies of the chromosomes can be clearly achieved even in the presence of allele drop out or loss of one region of a chromosome as in PB2 for chromosome 18 (m7-12), which is most likely caused by DNA degradation.

### (b) Example of Euploid Chromosome 14 and Aneuploid Chromosome 15 Due to a Meiosis II Error.

It was shown that while meiosis I and II (MI and MII) were correct for chromosome 14, a MIT error occurred after correct MI for chromosome 15 (FIG. 6b). Both remaining chromatids segregated into PB2 leaving the oocyte without any chromatid of chromosome 15. As a consequence the resulting zygote has a monosomy of chromosome 15. In this case PB1 and 2 were analysed with 4 independent markers per chromosome.

### (c) Meiosis I Error Resulting in a Trisomy of the Zygote.

Due to premature sister chromatid separation at meiosis I, only one chromatid of chromosome 17 segregated into PB1 (FIG. 6c). After correct MII with one chromatid in PB2, the oocyte remains with two chromatids thus leading to trisomy chromosome 17 after fertilisation.

(d) Repair of a Meiosis I Error with Resulting Euploidy.

No mistake was detected for chromosome 10 where PB1 has 2 positive PCRs for 4 markers and PB2 1 positive PCR. For chromosome 16 it was found that the opposite is the case: only 1 signal in PB1 and 2 signals in PB2 (FIG. 6d). This indicates that in MI only 1 chromatid segregated into PB1 leaving the oocyte with 3 chromatids at MII. The inventors predict that this MI error was then rescued by segregation of 2 chromatids into PB2 thus leaving the oocyte with a corrected haploid (in) chromosome 16.

### Materials and Methods

### Polar Body Collection, Cell Lysis and Limiting Dilution

The polar body is deposited in 30 μl of distilled water, frozen and kept until analysis at −20° C. or lower. The first step for single cell MCC is cell lysis and DNA preparation in a system approximating a closed system such that no material is taken from the original vial in which the PB is stored. 10 μl cell lysis buffer is added to the tube containing Triton X-100 (2%, 0.1% final concentration) Tween 20 (2%, 0.1% final concentration) and Proteinase K (20 μg/μl, final concentration 0.25 μg/μl), briefly mixed, overlayed with oil and incubated at 50° C. over night. Cell lysats (40 μl) are dispensed into 8×5μl aliquots, overlayed with oil and proteinase K is heat inactivated by incubation at 95° C. for 5 minutes.

Amplification with Seminested PCR

The protocol is similar to the one described in WO2007/129000 for MCC with genomic DNA. This method has been proven to be robust and to allow multiplexing at very high levels. The following represents a typical protocol; precise conditions (number of multiplexed markers; precise volumes and thermocycling conditions, etc) may be varied as appropriate.

The first round of PCR analysis is a multiplexed amplification step for each PCR well (i.e. aliquot) with all pooled outer primers in each PCR well, so that all copies of any target sequence are amplified to some extent. 5μl mastermix for the multiplex first round PCR is added and thermocycling is carried out with hot start at 93° C. for 9 min, followed by 25 to 50 cycles of 20s at 94° C., 30s at 50° C. and 1 min at 72° C.

The second round of PCR uses the product of the phase 1 multiplex PCR at a dilution of 1:100 in water as a template to amplify individual marker sequences on each chromosome as semi-nested PCR with internal forward and reverse primers in a volume of 10 μl. Thermocycling under oil is carried out with hot start at 93° C. for 9 min, followed by 33 cycles of 20s at 94° C., 30s at 52° C. and 1 min at 72° C. Prior to PCR analysis on 108-well horizontal 6% polyacrylamide gels 8 μl 2× loading buffer (15% w/v Ficoll, 0.1 mg/ml bromophenol blue, 4×SyBr Green I) is added and gels are run at 10V/cm for 10 min digital PCR analysis is performed by scoring presence or absence of PCR product in each sample.

The second round of PCR and digital PCR read out has been automated as melting curve analysis on the BioMark system of Fluidigm company. This system has proven most suitable and convenient as it provides the following set up:

(i) PCRs are run on a 96×96 well chip, which allows amplification of 96 DNA templates with 96 primer pairs. PCR run time is short (2.5 hours) and need of reagents is minute as PCRs are run in a 5 nanoliter scale;
(ii) digital PCR read out can be performed by melting curve analysis on the chip on the same platform within 45 minutes and results can be exported into excel databases which can be easily analysed; and
(iii) the automation procedure meets one important requirement for PB diagnosis, which is that the time for analysis should be as short as possible.

### Primer Sets

Primers are selected using various criteria after masking repetitive elements from the human genomic sequence (Ensembl database, NCBI release 37, retrieval of masked sequence; http://www.ensembl.org). Amplicon length of the external products is a maximum of 120 bp and the internal product between 75 and 100 bp. Amplicons were located such that they build two triplets (see above, under “Statistical considerations and error avoidance”) of linked markers per chromosome; on metacentric chromosomes 1 cluster on the short arm and 1 cluster on the long arm of the chromosome, in the case of acrocentric chromosomes the clusters were situated proximal and distal to the centromere. All primer sets were checked electronically against the reference genome to ensure that they were predicted to give unique products (http://www.ncbi.nlm.nih.gov/projects/e-per). Typically primer length is 18-20 bp with melting temperature of 52-60° C. Design requires at least two guanine or cytosine bases at the 3′ end and at least one at the 5′ end.

As many as 1200 primers have been multiplexed with robust results (Eichinger et al. (2005) Nature. May 5; 435(7038):43-57) therefore a marker set for an all-chromosomes-screen can easily enlarged by addition of more primers for disease specific sequences and mutations.

The primers used in this study are listed in Table 2 (see Appendix I). In this Table: Fex=external forward primer; Fin=internal forward primer; and Rvs=reverse primer.

### Fibroblast Production and Selection

The fibroblasts were remaining amniocytes after karyotyping. Fibroblasts at telophase were picked with a micropipette under a light microscope with 200× magnification.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

APPENDIX I
Table 2

| | | | Fex | Fin | Rvs | |
|---|---|---|---|---|---|---|
| Hsl007a01 | Chr1 | 48000696 | CATGAAGTTATGGGGTTAGG | GCTAGTTTCCTCTTGAAGG | CATGTGGCAGGCACATACG | CATGAAGTTATGGGGTTAGGTGCTAGTTTCCTCTTGAAGGAGAAA |
| | | | | | | CAGATAGTTTGAGTGTGTCAGCATGTTAGATGATGACCATATCGT |
| | | | | | | ATGTGCCTGCCACATG |
| Hsl007a02 | Chr1 | 48001391 | GAACCATCTCTTTCTTTCCC | CTCTGCATACACTTTTCTCG | CTGACCTCAGAGCTCATGG | GAACCATCTCTTTCTTTCCCTGTTTCATGCTCTGCATACACTTTTC |
| | | | | | | TCGCCCAGCTTAGAGTGTTAGCTTGGAGCATCCTTGTTTCAAGAC |
| | | | | | | CATGAGCTCTGAGGTCAG |
| Hsl007a03 | Chr1 | 48001519 | GCCAACAGAGACCTGACC | GTGTGGAATAGGTATGTTGG | GAGAACTTGCATCCATTTGC | GCCAACAGAGACCTGACCTGGTGTGGAATAGGTATGTTGGATAT |
| | | | | | | GCTTGTGAATGCCTGGCCAGGCAGGATGTGTTTTGAGGCTCACT |
| | | | | | | GCAAATGGATGCAAGTTCTC |
| Hsl007a04 | Chr1 | 48002656 | CGTGTTTACAGCCCTTTCC | CACAGGCCAAACAGGAAAGG | GCCTATTGCTTTGAGGAGC | CCTGTTTACAGCCCTTTCCAATTCACAGGCCAAACAGGAAAGGG |
| | | | | | | GGGAGGGGTTAGAGAAGGGCACAAATGTCAGAAATCACAAATCA |
| | | | | | | TACAGTTGCTCCTCAAAGCAATAGGC |
| Hsl007a05 | Chr1 | 48004064 | GAGCTTCTGTTGAGTGACC | GACTGGCTTCTTCTCTTTGC | CACAACAGGTGTTTGAGAGC | GAGCTTCTGTTGAGTGACCCATTGATAGACTGGCTTCTTCTCTTT |
| | | | | | | GCCCCAACTAGACCCCTCTGTGAGCTGTTTGTGCTGACCTTGGG |
| | | | | | | CTGGGAAGATGCTCTCAAACACCTGTTGTG |
| Hsl007a06 | Chr1 | 201000290 | CTTGGAGGCAGCATGTGG | GAGGTCAACCTCTAAAGTGC | GAGTGCTCCATTCACTACC | CTTGGAGGCAGCATGTGGGGAGAGGTCAACCTCTAAAGTGCCAG |
| | | | | | | CTCTCCAGAAATGCAGCCGGAATGAAGGTTTGAAGGGATGGTAG |
| | | | | | | TGAATGGAGCACTC |
| Hsl007a07 | Chr1 | 201000745 | CTACCCTCTAGTGATGAGG | CCCTTGGCCTGGAAAAGG | CAGCACCCCAAATCTGATCC | CTACCCTCTAGTGATGAGGGTCCCTTGGCCTGGAAAAGGGGAAG |
| | | | | | | GAGGAGATAGGGGGCTAGGCCTTGAAGGAAGTCAAACCCTAAGA |
| | | | | | | CAAGAGGATCAGATTTGGGGTGCTG |
| Hsl007a08 | Chr1 | 201001078 | CACCCTCCTTGGTAAGCC | GACACATGTAAACTGTCCC | GGTGTTTCCCCACTAGCC | CACCCTCCTTGGTAAGCCCCCATCCTAACCCTTTTGTGTGGTAAA |
| | | | | | | GACACATGTAAACTGTCCCAAAACAAAAGACAGAGAGCAGAGAC |
| | | | | | | TACCAGAGGGTGAGTGGAGGTACTTGGGTGGGTCTGGCTAGTG |
| | | | | | | GGGAAACACC |
| Hsl007a09 | Chr1 | 201001705 | GTTGGGCTGGTGCTTGGC | GTGTGCAAAGGGTTTCAGG | CTTCTTGTATTCTTGTGAGG | GTTGGGCTGGTGCTTGGCAGGGTGTGCAAAGGGTTTCAGGCCA |
| | | | | | | GATTAGTGGAGGTTGAGTGGGGATTGGAGGGTAGGGGTGGATT |
| | | | | | | GTCATGTGAGCCTCACAAGAATACAAGAAG |
| Hsl007a10 | Chr1 | 201002050 | CAGATGAGGAAACCAAAGGG | GAACATACAAGAGGGAATGG | GAAAGGCTGTCCTGAAACG | CAGATGAGGAAACCAAAGGGCAGAAACATTTTTAGGAGAACATA |
| | | | | | | CAAGAGGGAATGGGAATTTGTATTCTCCAAGTCCAGGGCCTCAC |
| | | | | | | TCTGCTGCACCCTGCACGTTTCAGGACAGCCTTTC |
| Hsl007a11 | Chr1 | 201002871 | GACACCATCACGTTTTCAGC | CTCAATACCAGAATCATCGC | CCAGTTGAGGAAACCAAAGC | GACACCATCACGTTTTCAGCTGACACTCAATACCAGAATCATCGC |
| | | | | | | TTGCCCCTTGTATTTGTGGCCAGTTTATTTTAAAAATGCTTCTGTG |
| | | | | | | CTTTGGTTTCCTCAACTGG |
| Hsl007a12 | Chr1 | 201003176 | GGTGACATGGTACTAGGG | CAGATGCCAGAAGAATGGG | CTGCTAGAGGAGACACTGC | GGTGACATGGTACTAGGGATCAGATGCCAGAAGAATGGGGGCAA |
| | | | | | | GACCTTGTGAAATAGGAGTTGGGGTTAAGGTCAGCCTTGTGTTG |
| | | | | | | GCAGTGTCTCCTCTAGCAG |
| Hsl007b01 | Chr10 | 9863702 | CATGTGAGTGGCTATACAAG | CAACCTAGGCTCAAAATGTG | GAACCTGCTGGAACTGAAG | CATGTGAGTGGCTATACAAGCCAACCTAGGCTCAAAATGTGCAG |
| | | | | | | TGATAGGGACTATTGCCTGTGATCACAAATTTTCGCATCTTTTATT |
| | | | | | | TTCTTCAGTTCCAGCAGGTTC |
| Hsl007b02 | Chr10 | 9863839 | GTCACTCTGAATATCTGAGG | GTGCATTAACGTGGAGCAC | CTCATAGAACTTATTGTGCTG | GTCACTCTGAATATCTGAGGTTCAGTGCATTAACGTGGAGCACAG |
| | | | | | | TGTTTGTTTTGAAAGTCATTCATGAAATAATAACCAATGTTCACCA |
| | | | | | | GCACAATAAGTTCTATGAG |
| Hsl007b03 | Chr10 | 9864315 | GGTAGAATAGAAAGAAACACC | GACATTTAGAAATGGCCTATC | CCAATATCCCTAAATCTCATC | GGTAGAATAGAAAGAAACACCAGGATATGACATTTAGAAATGGCC |
| | | | | | | TATCTTCAGATGTAAAGAACTATTTGGGTTAATTTTTTAATTGATAA |
| | | | | | | TTTGATGAGATTTAGGGATATTGG |
| Hsl007b04 | Chr10 | 9864801 | GGTATGTGAATCTATTTGCAC | CATGCTGAGTATTGTACAAAG | CATAGCCTTCTGTATGTTCC | GGTATGTGAATCTATTTGCACAAAGTAGCATGCTGAGTATTGTAC |
| | | | | | | AAAGCACACTAAACATCTTTTAAGTCACTTTGAAAATGGCAACAGT |
| | | | | | | CTCAGGGAGGAACATACAGAAGGCTATG |
| Hsl007b05 | Chr10 | 9864901 | GAACATACAGAAGGCTATGC | GCTATGTTGATACATCTAAGAC | CTTTGTTGCTTTTGTAATGGG | GAACATACAGAAGGCTATGCTGCTATGTTGATACATCTAAGACAA |
| | | | | | | CTGAGAGAAAAAAATATGCAGGGTAAAATAAACTTCCCATTACAA |
| | | | | | | AAGCAACAAAG |
| Hsl007b06 | Chr10 | 9865100 | CACTCAACCATTCAGTCTTC | GTTAGAATGAATGACTAAGCC | CAGGATTTAGTGGCTGATAG | CACTCAACCATTCAGTCTTCCAAGTTAGAATGAATGACTAAGCCA |
| | | | | | | TGATTCGTTTTTCTTGCTTTGATCTATCAGCCACTAAATCCTG |
| Hsl007b07 | Chr10 | 130654976 | CTTGTAAGTTCCAACATCTTC | GAGCATCAGTCAGTTTTAGC | GGGAATTTCTATAAGATGCAG | CTTGTAAGTTCCAACATCTTCAGAGCATCAGTCAGTTTTAGGAGT |
| | | | | | | GTCATTCTGAAGGCACTCCAAAGCCACCGCTGACTGCATCTTATA |
| | | | | | | GAAATTCCC |
| Hsl007b08 | Chr10 | 130655167 | CCTCAACAGCATGAATTAGC | CCAGATTCTTTACCTGCTAC | CACAATTCCTATCAAAGCTTG | CCTCAACAGCATGAATTAGCCCCAGATTCTTTACCTGCTACCAAA |
| | | | | | | GCTAGCCCAGAGGAAGAGGAACAGAGAGCGGACAAGCTTTGATA |
| | | | | | | GGAATTGTG |
| Hsl007b09 | Chr10 | 130655893 | GTAGACCAAAGGAAGAATGG | CTGCATCAGCTATTCTTTCC | GAGTCAGAAAACCATGACTC | GTAGACCAAAGGAAGAATGGAATCTGCATCAGCTATTCTTTCCTG |
| | | | | | | AACACAGACCCTAGAATATATTTTTTCTAGAAGTTTTTATATCATA |
| | | | | | | GTATCAAGAGTCATGGTTTTCTGACTC |
| Hsl007b10 | Chr10 | 130656501 | CAGCTGATCAAGTGAAGCG | CCTTTCCACAGACTATTGAC | GGTTCAAAGCGAAGACTATC | CAGCTGATCAAGTGAAGCGGCCTTTCCACAGACTATTGACGATCT |
| | | | | | | GTCTCAAGCATTATCTCATAAGTTTCCTTTTATTTTCTCTCCCAAC |
| | | | | | | CCAGATAGTCTTCGCTTTGAACC |
| Hsl007b11 | Chr10 | 130656892 | GATCTTCATGGACACAAGTC | GTCTGTGAAGATAAAGGAAAG | GGATTAGACCTATTTGTTGAG | GATCTTCATGGACACAAGTCTTGTCTGTGAAGATAAAGGAAAGTA |
| | | | | | | AAATCACTTATGCAAAAGTAGATATTTGTGACAGACTCCTGGATG |
| | | | | | | GACCTCAACAAATAGGTCTAATCC |
| Hsl007b12 | Chr10 | 130657597 | GATGAGTGCAGATTTGAAGG | GATACAAGATGTGAACATTGG | CGGAACAATTACAAGTAAAGC | GATGAGTGCAGATTTGAAGGGGAGATACAAGATGTGAACATTGG |
| | | | | | | AAGCAACCACCATAGGATTCATTACATCAATCATGGATGCTTTAC |
| | | | | | | TTGTAATTGTTCCG |
| Hsl007c01 | Chr11 | 36000088 | CAGCTTTGCTTTGCTTGGG | GTGATTCTGACCCAGTACC | CATGAGGCTAAGAAAACAGC | CAGCTTTGCTTTGCTTGGGACATGTGATTCTGACCCAGTACCCCA |
| | | | | | | GACCTGAAGGCCCCTCTATGTGTCAGTCCTGAAAGGATTCGCTG |
| | | | | | | TTTTCTTAGCCTCATG |
| Hsl007c02 | Chr11 | 36000805 | GTGCTGCATTAGAGTTTGG | CTTGACTAGGTGGAAGAGC | CCAAGGGGATCAAGCAAGC | GTGCTGCATTAGAGTTTGGTCACAGGCTTGACTAGGTGGAAGAG |
| | | | | | | CTTTCTGAGAGTTGTGTGCAAAAAAACACTTAGCTGCCGTTCCAT |
| | | | | | | ATTTGCTTGCTTGATCCCCTTGG |
| Hsl007c03 | Chr11 | 36001041 | GTATGATAGAGTTTTCCTTCC | GTTGACCATGGCTTAGTCC | GAGACAGACAGTCTCAACG | GTATGATAGAGTTTTCCTTCCTGAGGTTGACCATGGCTTAGTCCT |
| | | | | | | TGCTATACAGGGTAGTGTGAGGATTGGATTTCTCAGGTTAGCGG |
| | | | | | | AGCTTTAGACGAGCGTTGAGACTGTCTGTCTC |
| Hsl007c04 | Chr11 | 36002360 | CAGATGTGTTTTGATTTCAGC | GTCAATTGCCCAGTGTTTAGG | GGGGTCCCCAGACTGTGG | CAGATGTGTTTTGATTTCAGCCAAGAACAAAGATATTTGATATGTC |
| | | | | | | AATTGCCCAGTGTTTAGGAAAAAGGATAATTTTGGTTACTGCTTTT |
| | | | | | | GAACTAGTGGTGGGAACCTTGGAAATCCCCCACAGTCTGGGGAC |
| | | | | | | CCC |
| Hsl007c05 | Chr11 | 36002551 | GCAAGACTTCCTCGTTTGG | GGTTTTCAGATTGGTTGGG | GCTGTAAGTGGACCATGGC | GCAAGACTTCCTCGTTTGGATTTTGGTTTTCAGATTGGTTGGGGG |
| | | | | | | AAATCTCACATACGGGAAGAAGAAGAAAAACAAATATAAGTAAGT |
| | | | | | | TTCCCTTTGGGGCCATGGTCCACTTACAGC |
| Hsl007c06 | Chr11 | 36003225 | CTGCAGTTTGCCAAAGTCG | CAGGATAGACTTGGAAATGC | CTACAGCTGGTTCCTGTCG | CTGCAGTTTGCCAAAGTCGCATTGGCAGGATAGACTTGGAAATG |
| | | | | | | CAAGGGTGCTTGGCATCTCCCATCAAGTTGGCATTTCCCTGGCTT |
| | | | | | | TAGCTTTCGACAGGAACCAGCTGTAG |
| Hsl007c07 | Chr11 | 118001364 | CTTGCAGGCCATGGAAGG | CCTACATCTTTCCTGTTAGC | CACTGTAGCAGTAGAGCGC | CTTGCAGGCCATGGAAGGGGACCCTACATCTTTCCTGTTAGCAC |
| | | | | | | TGCGGGTGGCTTTGTTTAAGCAATGAGCTATGAGAGAACATCTCC |
| | | | | | | CCTCCTGCTGTGTGCGCTCTACTGCTACAGTG |
| Hsl007c08 | Chr11 | 118001547 | CTGGAGCTCCTGAATTGGG | GGTCTTCATCTTTCTCCGG | GACTTTGCTTTACAATCTTTGG | CTGGAGCTCCTGAATTGGGAGGGTCTTCATCTTTCTCCGGCTTCA |
| | | | | | | ACCTTAAGTCTGCTCTCCAAATGACTTGATAACACCATAGGAACC |
| | | | | | | AAAGATTGTAAAGCAAAGTC |
| Hsl007c09 | Chr11 | 118002383 | GATGCACCTGTGCTATTGC | GTTAGGAGGCATGGATACC | GATTGGGTCGATTGACTCC | GATGCACCTGTGCTATTGCCTCCTCTGTTAGGAGGCATGGATAC |
| | | | | | | CCCCCAGCCTCCTGGAAAGCTGAACATAGGGAGTTAAAGGGTTG |
| | | | | | | TTCTCCACCGGGAGTCAATCGACCCAATC |
| Hsl007c10 | Chr11 | 118003606 | GCAAACACCTACACGTTGG | CATATCCCCAGTTCCTTCC | CTTCACATGAACGCCTACC | GCAAACACCTACACGTTGGTACATATCCCCAGTTCCTTCCCAGGC |
| | | | | | | ACTGGCCTTATGCCCAGCACCCGGAAACTCTTTGGAAGGTAGGC |
| | | | | | | GTTCATGTGAAG |
| Hsl007c11 | Chr11 | 118004318 | GGTATTGTTGTCATCCAAGC | CATGCATAAGATAGTCAAAAG | GCTTTACTTTACTTTGTCCC | GGTATTGTTGTCATCCAAGCCAGAGGAATAAACCATGCATAAGAT |
| | | | | C | | AGTCAAAAGCACTGCATATCAGGTGGGAGGTGGGAGGGTAGGG |
| | | | | | | ACTCCAACCTGGGACAAAGTAAAGTAAAGC |
| Hsl007d2 | Chr11 | 118004587 | CTCCAGATGCCTCAACAGG | GCTCAGGCCAAGAAAGACG | GTAACTGTGGAGTGGATGG | CTCCAGATGCCTCAACAGGCATAGCTCAGGCCAAGAAAGACGGC |
| | | | | | | TCCTCAAATGTCCAGCATCTGCCCATCATGCATCACCCCTTACAT |
| | | | | | | GCAGAGCCCATCCACTCCACAGTTAC |
| Hsl007d01 | Chr12 | 25000837 | CACAAAACTAAAGTTGACTCC | GTCTTTGCCAACTCAACAGG | CTCCTCCTATGCTTCTGACC | CACAAAACTAAAGTTGACTCCAAATGTCTTTGCCAACTCAACAGG |
| | | | | | | ATAATATTAAATGCGGAATATTTTGTTCCCCTTGTACCTCTCCAGG |
| | | | | | | TCAGAAGCATAGGAGGAG |
| Hsl007d02 | Chr12 | 25001003 | GTTACCACCTTCCCTCTTGC | GAGTTCAATACTTTCTTCTCC | CTCAGGTGGACTATGATCC | GTTACCACCTTCCCTCTTGCCATTTTTAATTTATGAGTTCAATACT |
| | | | | | | TTCTTCTCCGGTCTCTTCCTTTCCTAAGAGATTTCAAGTCAATTTC |
| | | | | | | CATGGATCATAGTCCACCTGAG |
| Hsl007d03 | Chr12 | 25001651 | GAGTTTTCAACCTGGCTAGC | GGACACAGGAAGGTGTGC | CTCAATGGGTAGAGAAATCC | GAGTTTTCAACCTGGCTAGCCTAGGACACAGGAAGGTGTGCTCT |
| | | | | | | AAGCCAGAAGGAGAATAGACTTCCTAGTTTTAATGCACTCCATTT |
| | | | | | | GGATTTCTCTACCCATTGAG |
| Hsl007d04 | Chr12 | 25002003 | CTGAGTATGCAAACAGCACC | GATACATGCAAAGCAAGAACC | GGACTTGGCCATGAGTTGG | CTGAGTATGCAAACAGCACCATTTGATACATGCAAAGCAAGAACC |
| | | | | | | CATGCTGCTTAAACCAGTTATTCTCGTTCACCCATAGGGGCATTC |
| | | | | | | CCAACTCATGGCCAAGTCC |
| Hsl007d05 | Chr12 | 25002740 | CAGTTCCACCTTTCCAGGC | CTTACATACTTGGGATTGGC | GCTCTTTGTACTCTTGAGC | CAGTTCCACCTTTCCAGGCTCTTACATACTTGGGATTGGCCCACA |
| | | | | | | GGGACACTGGATTAAAGGTTCCACTTGAAAAATAAGGTCCCACTG |
| | | | | | | GGCTCAAGAGTACAAAGAGC |
| Hsl007d06 | Chr12 | 25003293 | CATCCTTCTGTTTCATAGCC | GACTGCTTCAGGACATGGC | CTACCTGCTAGTTGATGTGG | CATCCTTCTGTTTCATAGCCTAAGTGACTGCTTCAGGACATGGCA |
| | | | | | | GGGTCTTCAGGAGGTGGTAGGTGCAGGCGAATGTGTCATTAGCA |
| | | | | | | CACCTGCCCACATCAACTAGCAGGTAG |
| Hsl007d07 | Chr12 | 58000080 | GTTTCCTTCATTCCATGTTCC | CCATTCTTAGTAACCTATACC | CTTCTCCCAATTCCCATGG | GTTTCCTTCATTCCATGTTCCAAGTAATGCCATTCTTAGTAACCTA |
| | | | | | | TACCCAGGTTTCTGTCTCTGTTCCATGGGCTGCTGGGTTGGGGG |
| | | | | | | CCATGGGAATTGGGAGAAG |
| Hsl007d08 | Chr12 | 58000698 | CAAAGTGACTGTGTCCAAGC | CTTCCTGAGCAAAGAGACC | CATAGATGTCAGAAGTCTCG | CAAAGTGACTGTGTCCAAGCCCGTGTGGGACTTCCTGAGCAAAG |
| | | | | | | AGACCCCAGCCCGGCTGGCCGGGCTTCGGGAGGAGGACCGTGT |
| | | | | | | GTCCATCCTCATAGATGGCGAGACTTCTGACATCTATG |
| Hsl007d09 | Chr12 | 58001335 | CCCAGCTATGAGAAGTACG | CACCATTGTCATCCAGTACG | GCTTGGGGAAAGCCAAAGG | CCCAGCTATGAGAAGTACGGCACCATTGTCATCCAGTACGTCTTC |
| | | | | | | CCGCCCGGTGTCCAGGGGGTAAGAAGACCATGGCCTGCCCTTA |
| | | | | | | CCCTTTGGCTTTCCCCAAGC |
| Hsl007d10 | Chr12 | 58001802 | GAGTAGTCAAGGCCTATAGG | CTGGACAAAGAGTAATGTGC | CCACTGTCTAACTTGTTCC | GAGTAGTCAAGGCCTATAGGTGTCTTCCTGCTGGACAAAGAGTA |
| | | | | | | ATGTGCAATTCTGGCTGCAGAGGGGTGAAGAAGCTGCACAGAAG |
| | | | | | | AGTGATGGAAAATGGAACAAGTTAGACAGTGG |
| Hsl007d11 | Chr12 | 58002815 | GTTCAAACAGCTAACAACCC | CTTTGCTCCCAGGTTTGGG | CTTCAGTCATCTGTGATACC | GTTCAAACAGCTAACAACCCTCACCCTCATTTCTCTTTGCTCCCA |
| | | | | | | GGTTTGGGTACCCAGACCCCACCTACCTGACCCGGGTGCAAGA |
| | | | | | | GGAGCTGAGAGCGAAGGGTATCACAGATGACTGAAG |
| Hsl007d12 | Chr12 | 58003132 | CACTCCCTTCTGGCAGAGG | CTCCAAGGCTCTGTTCTCC | GTGGAGCACAGCACATACC | CACTCCCTTCTGGCAGAGGCCGACCTCCAAGGCTCTGTTCTCCC |
| | | | | | | CTCCCCGTGTACATATACTCCCGGTTTCCCTGCCCCTCCATTGCC |
| | | | | | | CTTGGCTTTTTCTGGTATGTGCTGTGCTCCAC |
| Hsl007e01 | Chr13 | 21000889 | CAATGTCTCCTAACAGTTGG | GTCTGAAGTAAAGCTCAACG | CTTGATTTGTCAGGGTGGG | CAATGTCTCCTAACAGTTGGCAGACATGTCTGAAGTAAAGCTCAA |
| | | | | | | CGATGAAGTTCTGGAATCTCAGGGCCCCATCCAGATGCCCCAGA |
| | | | | | | CCACACCCACCCTGACAAATCAAG |
| Hsl007e02 | Chr13 | 21001057 | GGATGACATCATTCCGAAGG | GCAGAACCCAAGGTCAGC | GGGAATGAATCTGCAACCC | GGATGACATCATTCCGAAGGACAGGCAGAACCCAAGGTCAGCAA |
| | | | | | | TTTCCGAAGCTCATCACCACCAACTCACACCAGCAGGCTGAGAA |
| | | | | | | CCTGCCGAGGGTTGCAGATTCATTCCC |
| Hsl007e03 | Chr13 | 21001506 | CACTCCCTTGGCTATCCG | CCATTCTACCCCACGAAGG | CATCCTGGGCTATGAGACG | CACTCCCTTGGCTATCCGGGTGTCCATTCTACCCCACGAAGGTC |
| | | | | | | TAAGGGCTTACAGAGCTGCAAGGGAACAGAGAGAGAATGGGTGA |
| | | | | | | TGACAGGGGAGCGTCTCATAGCCCAGGATG |
| Hsl007e04 | Chr13 | 21003103 | GCTGTCAAACTTCAACTTGC | GAGGATCCTGAAACAGAAGC | GTGAATGGAATGAGCATTGG | GCTGTCAAACTTCAACTTGCTTTATGAGCCCAGAGGATCCTGAAA |
| | | | | | | CAGAAGCGCCCACACCAGTGAGTCCTAGAGGAGCAGTGAGTCCT |
| | | | | | | AGTTGCCCCCCGACCAATGCTCATTCCATTCAC |
| Hsl007e05 | Chr13 | 21004004 | GCAAGGGTCAAACTTCAACC | CTACTGGAATGCTGGCACG | GCTGACCTTGACCATCACC | GCAAGGGTCAAACTTCAACCTGCTACTGGAATGCTGGCACGCTG |
| | | | | | | GTTGTGACCTTGCTCCTGAAGTAGCTGGCCAACGGAGGTGCTGC |
| | | | | | | CACTGAGCGGTGATGGTCAAGGTCAGC |
| Hsl007e06 | Chr13 | 21004702 | CTCCTCCAGCAGCAAAAGG | CACCAGAGTCCTCCATGG | GAAGGGTTTGGGATTCTGG | CTCCTCCAGCAGCAAAAGGAAACACCAGAGTCCTCCATGGCTCT |
| | | | | | | TGCAATGGAGAGTTCTTTGTGTACACCTCCCACCCGATCCCCTTA |
| | | | | | | CACCAGAATCCCAAACCCTTC |
| Hsl007e07 | Chr13 | 107000502 | CCATACTTTAGATAGGTTACC | CCACAAAAGAGACCATAGGG | GAAGCTGTCAAATGACTAATG | CCATACTTTAGATAGGTTACCTATATTGTTACTGCCACAAAAGAGA |
| | | | | | C | CCATAGGGCTCATAGCAACAGAGGCAGAATAAACGCCTCAGTGA |
| | | | | | | GATTCCAAGAGCATTAGTCATTTGACAGCTTC |
| Hsl007e08 | Chr13 | 107001062 | CCAGAGACTAAGTCAGAAGC | CTGTAGCATAGATCATGGG | GCGAATGCAGAGAAACAGC | CCAGAGACTAAGTCAGAAGCATTTTAGTTTAAATACTGTAGCATA |
| | | | | | | GATCATGGGACATAACCCAGGCATGGAATATATATACTTCAAAAC |
| | | | | | | TATCCCTGCTGTTTCTCTGCATTCGC |
| Hsl007e09 | Chr13 | 107002082 | GTGCAAAGCAAGCATCAGG | CTTTGTTGGCTTTCCAATTCG | GGCATTGCAGATATGTGCC | GTGCAAAGCAAGCATCAGGGTTGCCTTTGTTGGCTTTCCAATTCG |
| | | | | | | TTGCCAGCAGAAGCCCATGTGATAAGAACTTTTTGATTAAGCTCT |
| | | | | | | AAATCTTTGGCACATATCTGCAATGCC |
| Hsl007e10 | Chr13 | 107002701 | GTATCAAAGGCAGTGGAAGC | CGCTCCCTTCCTATGATCG | CAAGCACTGTTTGTTCAAGG | GTATCAAAGGCAGTGGAAGCTGGGCAACGCTCCCTTCCTATGAT |
| | | | | | | CGGTGTGTGAGCCCTGACTTAATGAGCTCCTACTAGAGGTGCTA |
| | | | | | | CCTTGAACAAACAGTGCTTG |
| Hsl007e11 | Chr13 | 107003344 | CATTCTGCAACTGCTTTTCC | CTCACCACAAACCTCATGG | GGAAACAACAGGATCATAGG | CATTCTGCAACTGCTTTTCCTAGCTCACCACAAACCTCATGGTTG |
| | | | | | | TATCTCTTTGTCTTTTGGACTCGGATTCTTCAAGCACTCGAATCCT |
| | | | | | | ATGATCCTGTTGTTTCC |
| Hsl007e12 | Chr13 | 107003766 | GTGTTTGTAGGGTCCCACG | GCAGAGCAGAAATCACTACC | GGAGATTGCTAATGATTTGC | GTGTTTGTAGGGTCCCACGTAAGCAGAGCAGAAATCACTACCGC |
| | | | | | | TGATCAAGGAGAGATGAACAGCATCACTAAACAGTGTTCAGAGA |
| | | | | | | CTTAGCAAATCATTAGCAATCTCC |
| Hsl007f01 | Chr14 | 30000501 | GGAACATCTCTGCATACAGG | CTTCCACCTCATGACTAGC | GAGACAGTGACCAGATCGG | GGAACATCTCTGCATACAGGTGTTAAAAGAAGCTTCCACCTCATG |
| | | | | | | ACTAGCATAAAACTTAAACCAATGGTTGTTATTCAGCTGAAGACA |
| | | | | | | GTATCAGTGTAAAGTGCCACCGATCTGGTCACTGTCTC |
| Hsl007f02 | Chr14 | 30001001 | CATTCCCTAACCCCACAGC | CAAAGCTTTCCTGTACACC | CACCTCTCAGTGGATAGGC | CATTCCCTAACCCCACAGCTCAAAGCTTTCCTGTACACCTGCTCT |
| | | | | | | ACTCAGCTCATCAATTTTCTGTGAGCCAGTTAAGTTCCTTTAAGC |
| | | | | | | CTATCCACTGAGAGGTG |
| Hsl007f03 | Chr14 | 39001001 | GTAGAAGCTTCTTTTCTTAGC | CAACACAGCCTGCATCTCC | GACCTCAAGTCATGGTAGG | GTAGAAGCTTCTTTTCTTAGCCAAAGAAACAACACAGCCTGCATC |
| | | | | | | TCCAGTGTAATGCCTTGACCAAACATGGAAATAGCAATGATAGGG |
| | | | | | | AATCAGTGCCTACCATGACTTGAGGTC |
| Hsl007f04 | Chr14 | 39002501 | CTAGAAGAGAAACTACAAGC | CTCAAAGCTGGGGTAACG | GGTTTGAAGAACTTACCAAGC | CTAGAAGAGAAACTACAAGCTGCTTAATCTCAAAGCTGGGGTAAC |
| | | | | | | GTAAGTAAAGTGCATTCAGGTCGAAGCCTGGAGGAGAGATGACC |
| | | | | | | TGAAGCTTGGTAAGTTCTTCAAACC |
| Hsl007f05 | Chr14 | 39003001 | GGGTACAATGAACTGTAATGG | GAGATACTCCTGAGATGGC | CAGACATTACTAAAGAACGC | GGGTACAATGAACTGTAATGGTGAGATACTCCTGAGATGGCAGC |
| | | | | | | CTTCAGAAAAGACTTTTTGACACATAAAGCTTGTCGATACTGACC |
| | | | | | | CTTGTTTGTAAGCGTTCTTTAGTAATGTCTG |
| Hsl007f06 | Chr14 | 39004501 | CAAGGATGCAACACTGAGG | GCTCCCAACAGGCATTACC | CTTCAGAATTCTTCAACATGG | CAAGGATGCAACACTGAGGTGGGGCTCCCAACAGGCATTACCCC |
| | | | | | | AGCAAATGAGGCCAAAGACCACAGCTAAAGTGATCTTAACCATGT |
| | | | | | | TGAAGAATTCTGAAG |
| Hsl007f07 | Chr14 | 82002001 | CTACCCTTTCTCCAACTGC | CTTGCTTCTTTCACTTAGCC | GGTTGGAGAAGTGTGATCC | CTACCCTTTCTCCAACTGCCCTTGCTTCTTTCACTTAGCCATAACT |
| | | | | | | CTGGCATCCTTCCCAATTTCATTCACATTTCGTCTTGGATCACACT |
| | | | | | | TCTCCAACC |
| Hsl007f08 | Chr14 | 82002501 | GAGCTGCTAGAGCTTTTGC | CAGCAATGAGTAGCTGACG | GAACCACTTTGGAGACTTGG | GAGCTGCTAGAGCTTTTGCCTTTAGCCAGCAATGAGTAGCTGAC |
| | | | | | | GTGCTCTGAGAATTCTCATAGGACCTGACTTCCTGGGGAAGTTC |
| | | | | | | CAAGTCTCCAAAGTGGTTC |
| Hsl007f09 | Chr14 | 82003001 | GAACCTGAACGTGTTGAGG | CAACTTGCTTTTCACTTAAGG | CTAGAGTTGGTGACAATTGC | GAACCTGAACGTGTTGAGGACATAAATCCAACTTGCTTTTCACTT |
| | | | | | | AAGGATGGTGAGACAACCTCCAGAGACTTTTCCTGAGAATGGGG |
| | | | | | | CAATTGTCACCAACTCTAG |
| Hsl007f10 | Chr14 | 82003501 | CAGATCATAGATTGTGGAGG | GATCTACCTAATGTTTGAAGC | GAGCAAATGTCACCTCACG | CAGATCATAGATTGTGGAGGAGTATGTTTGATCTACCTAATGTTT |
| | | | | | | GAAGCTGATAGAAGATGAAAGGGGGGAGGGAGCCTCAGGCTGT |
| | | | | | | TTACCAAGTTTCATCGTGAGGTGACATTTGCTC |
| Hsl007f11 | Chr14 | 96001879 | CTGGAGTAGAGTCTGGGC | GGTGTAGTTGATTTCACTGG | GAAGTGAGGATAAGTGAACC | CTGGAGTAGAGTCTGGGCTGAGGGTGTAGTTGATTTCACTGGGT |
| | | | | | | CTTGAGGATCTGGGGCTCTGTACTGTTGCCAACTTGAGCAGTAG |
| | | | | | | GTAAAGTCCTAAAGGTTCACTTATCCTCACTTC |
| Hsl007f12 | Chr14 | 96003169 | GGGTGGGACCTAGAAAGC | GAGTTGAGGAGTCGAGAGG | GTTGACAAGGAAGACAAAAGG | GGGTGGGACCTAGAAAGCATGTTGAGTTGAGGAGTCGAGAGGG |
| | | | | | | CAGGTTCAAATTACCACGTATATGTAATATTACCATGTGTTATTCT |
| | | | | | | CATGACCCTTTTGTCTTCCTTGTCAAC |
| Hsl007g01 | Chr15 | 61000243 | CTAACTGTCACCTCCTTGG | CTGAGGCTTAGAGTTTAGGG | CTCCTCTATTGCCAGAATGC | CTAACTGTCACCTCCTTGGACTGAGGCTTAGAGTTTAGGGTTTTC |
| | | | | | | AGGATAGAGAGCTTATCTGTTAGGTCCTTTGAACCGCTCCCTAGC |
| | | | | | | ATTCTGGCAATAGAGGAG |
| Hsl007g02 | Chr15 | 61000843 | CATAGAAATCCTAACATCTTCC | CCCAAGCCTTTTCAGTTCC | GAATACCAAACAGACTTAGC | CATAGAAATCCTAACATCTTCCCCTCCCTCCCAAGCCTTTTCAGT |
| | | | | | | TCCCTACACTTTCCCCCCAACCCTGTTCCCAGGGTATAGCGGCA |
| | | | | | | ATAGAGCAGCTAAGTCTGTTTGGTATTC |
| Hsl007g03 | Chr15 | 61001150 | CAAGGCCTTGATGTAGTGC | CTAGCAAAGAATACGTGAGC | GTTTCCTGAAGGCCTCTGG | CAAGGCCTTGATGTAGTGCCTGCATAGCTAGCAAAGAATACGTG |
| | | | | | | AGCAGCTAGTCATTCCTATCCTAGGGAAGCTCCTGAGCCCATGA |
| | | | | | | GCATGGGGAAAATCCCAGAGGCCTTCAGGAAAC |
| Hsl007g04 | Chr15 | 61002774 | GTAACCCGTCTAAGATGTGG | GGATATGTTCAAGTCTCAACC | GCATGCCAGGTGAAGGCC | GTAACCCGTCTAAGATGTGGTGCAGGATATGTTCAAGTCTCAACC |
| | | | | | | CAGGCAAGAGCTCTGTGATGAGAAGTTGACTATTAATGGCTGGG |
| | | | | | | TGGCCTTCACCTGGCATGC |
| Hsl007g05 | Chr15 | 61003364 | CCCTGCTTTGAGTAACTCC | GTCTCCGTGCCCTCAAGG | CAGTTTAGAAGTAGGAGTGC | CCCTGCTTTGAGTAACTCCCAACACAGTCTCCGTGCCCTCAAGG |
| | | | | | | CTATGTGTATTTCTCACTTTCCCTGGAACTAGTCACTCATGGACA |
| | | | | | | CTCTGCACTCCTACTTCTAAACTG |
| Hsl007g06 | Chr15 | 61003869 | CTATCCTTCAGTTTTCTAACC | CTGTCTCTTTTGGTCCTACC | CTGGAGGTCCAATCAAAGG | CTATCCTTCAGTTTTCTAACCTTCTGTCTCTTTTGGTCCTACCTTC |
| | | | | | | AGCTCAAGGGCTTAAGAAAGAAGATATTTCTTTTGGGGAAGATGA |
| | | | | | | TTTAACCTTTGATTGGACCTCCAG |
| Hsl007g07 | Chr15 | 61004786 | GATAGGACCCAGTGTATTGC | GCATTACATGACGGACTGG | GTGCAGTTTGCAAGAAAGGC | GATAGGACCCAGTGTATTGCAAGGCATTACATGACGGACTGGAC |
| | | | | | | CCAATTCAAGCTCTGGTACTTGTTCCCGAGGCCAGAAGACAAGC |
| | | | | | | CTTTCTTGCAAACTGCAC |
| Hsl007g08 | Chr15 | 93000191 | CTAAGACGAAGTCCTCAGC | CTCCAATACTGCAGAGATGG | CGGCTGTCCTTTCTTTGGG | CTAAGACGAAGTCCTCAGCTCTCCAATACTGCAGAGATGGTGTCT |
| | | | | | | CATTCTGAGATATCCTGCAGCACACCAGAGGCCTCAAGAGTGTT |
| | | | | | | CCCAAAGAAAGGACAGCCG |
| Hsl007g09 | Chr15 | 93000752 | GTGCACTGTCAATACAACG | GGATGCACCCAGCTAACC | CCTTTCCTTAGGATAACAGC | GTGCACTGTCAATACAACGTCCCGGATGCACCCAGCTAACCTCA |
| | | | | | | TTTGGGAAGGCAAAATTAATTAGTTTGTGTTTTAACACCCAGCTGT |
| | | | | | | TATCCTAAGGAAAGG |
| Hsl007g10 | Chr15 | 93001078 | GAGCTCTGGATTCATTCCG | CCTCATTTGCTGTTAACACC | GGACAGGAATAGAAATGCC | GAGCTCTGGATTCATTCCGGAGCCTCATTTGCTGTTAACACCTTT |
| | | | | | | TCCAGTTAGCAATTCTGGGTGAAAAGCCTGGCCCCAGATCTGAG |
| | | | | | | AGGTTGGGCATTTCTATTCCTGTCC |
| Hsl007g11 | Chr15 | 93001829 | GCAGTCATAGTTCTTGAGG | CCTCAGCACAGAGGCAGC | CCAGTCTTATGCATTGTGC | GCAGTCATAGTTCTTGAGGCCCTCAGCACAGAGGCAGCAGGACC |
| | | | | | | AACGACCTTCCCAGGAGCCCACAGATCAGCGGGAAAGGCAGGT |
| | | | | | | GTGCACAATGCATAAGACTGG |
| Hsl007g12 | Chr15 | 93002103 | GCTGATGGTAATCATCTGG | GTGGTTAACAGTCTGACTGG | GAAACTAAGCACGTGCATCC | GCTGATGGTAATCATCTGGAGGTGGTTAACAGTCTGACTGGGGA |
| | | | | | | GATGACAGTAGAACAAAGGCAATATTTCCAGGAAGACAGGATGC |
| | | | | | | ACGTGCTTAGTTTC |
| Hsl007h01 | Chr16 | 52000016 | CATCTGTCAGCAAACTGTTCC | GGCAGACCCAATTCTTAGC | CAGTCTTTGGTAGACGATGG | CATCTGTCAGCAAACTGTTCCAGGCAGACCCAATTCTTAGCACCA |
| | | | | | | CAATAAAATGAAGGACATCAGGATAATCCATCAAACAAAAGCAGC |
| | | | | | | TGGGAGCACCATCGTCTACCAAAGACTG |
| Hsl007h02 | Chr16 | 52000747 | CTATGGGTATGATATGTTCGG | CCTACAGCAATACTTTGTCC | GTAGCCACAGGTGGCACC | CTATGGGTATGATATGTTCGGCCCTACAGCAATACTTTGTCCTCC |
| | | | | | | TACATTATTTAAGCAGAGCTCATTAAGGGACTGGACAACCAGATG |
| | | | | | | ACAGCCCAGGGTGCCACCTGTGGCTAC |
| Hsl007h03 | Chr16 | 52002355 | GTAGGGAACATGCAAATCCC | CTGTTCTGTTCTACATTCACC | CTTCCATTCTGTAGGGAGG | GTAGGGAACATGCAAATCCCTCTTCTGTTCTGTTCTACATTCACC |
| | | | | | | CCCCAGAGCATTCTGGATGCTTCTCAGAATTTCCAAATCCTATTC |
| | | | | | | ATCCCCTCCCTACAGAATGGAAG |
| Hsl007h04 | Chr16 | 52002765 | CAACCACTATGTCACAAAGC | CAAGAACAGAGCCCATGGC | GCTCATTTCCTGTAAACAGC | CAACCACTATGTCACAAAGCCCAAGAACAGAGCCCATGGCTGAC |
| | | | | | | TGAAGTCAGCAGTTGCAATCAGGATAATTCTGTAACTGAATAATG |
| | | | | | | CATGCTGGAATGCTGTTTACAGGAAATGAGC |
| Hsl007h05 | Chr16 | 52003709 | GTCTTCATCCATCAGACTGG | CCAGCTCCCCATGAAGGC | GGAGACTATGCATCTTTCC | GTCTTCATCCATCAGACTGGACCAGCTCCCCATGAAGGCTGAGA |
| | | | | | | AAATAGTCAAGTAAGAAAATAGGAGGGTAGCCAAGACCGGCTGC |
| | | | | | | CCTCTCTAGGAAAGATGCATAGTCTCC |
| Hsl007h06 | Chr16 | 52004798 | CACTCAATAGACTTTCAGGG | GCAATAGCTCAGGCAAACC | CTGGTCAGTGGGCAGCCG | CACTCAATAGACTTTCAGGGAAATGCAATAGCTCAGGCAAACCTT |
| | | | | | | GCTTACCTCAAACTTTTACTAAGCAAATAAACAGATTTTGAAAGTC |
| | | | | | | GGCTGCCCACTGACCAG |
| Hsl007h07 | Chr16 | 79000135 | GAGGCTATAGGTTAAGAGG | GCTCAGAAACAAATCATTTCC | GGGGTGTACAGTAAACGG | GAGGCTATAGGTTAAGAGGAGATAACAGACATGCTCAGAAACAA |
| | | | | | | ATCATTTCCTGATAGCTGTTTCAGATGGAACCAAATGGAAAACAG |
| | | | | | | TGCTTCTTTCGTTTACTGTACACCCC |
| Hsl007h08 | Chr16 | 79000890 | GTCTCCACTGGAAGAAGAGC | GCAGACTATTCAAATGCTTCC | CAGATGCATGACTATGGGG | GTCTCCACTGGAAGAAGAGCCTGTAGAATATGCAGACTATTCAAA |
| | | | | | | TGCTTCCTTGGTCCATTGTTGTCCTTTCTTTTCTCTTCTAGAACT |
| | | | | | | TTCCCCATAGTCATGCATCTG |
| Hsl007h09 | Chr16 | 79001030 | CCTGTTTTCCCAAGTTTACC | CTCTGAGAAGCCCATCAGC | GGCTAGATTCATCCACTTGC | CCTGTTTTCCCAAGTTTACCTGCCTCTCTGAGAAGCCCATCAGCC |
| | | | | | | CTGAGAGATACCTGGAAGGAAAGAGGAAAATGCGTGATTCAAAT |
| | | | | | | CATGTTGCAAGTGGATGAATCTAGCC |
| Hsl007h10 | Chr16 | 79001583 | GGTGTTAGGTTCCCACAGG | GAAGGATCACCATGAACGG | CAAAGATTTGGAACTCTGTGC | GGTGTTAGGTTCCCACAGGATGAAGGATCACCATGAACGGTCAG |
| | | | | | | GACCTGACTTAGGAGGACTCAGAAGCTGGAGACTGCAGAGGATG |
| | | | | | | GCACAGAGTTCCAAATCTTTG |
| Hsl007h11 | Chr16 | 79002130 | CAAGTGAATGAGTGAATGGG | GACTATCCAGAAACTGTGC | GCTCAGAGCACATGGTTCC | CAAGTGAATGAGTGAATGGGCGATTTCCAGACTATCCAGAAACT |
| | | | | | | GTGCCCCATAGTCCTACCCGTAGGAATCCAACAGGGAACTGTCA |
| | | | | | | CCACCGACCCGAGGCAGGAACCATGTGCTCTGAGC |
| Hsl007h12 | Chr16 | 79003012 | GAGCAGTCAGGGGACTCC | CTGTGTCTGGTCTTATGGG | CTTTGTCCCCTGAGGTAGC | GAGCAGTCAGGGGACTCCCTGGCTGTTTCTGTGTCTGGTCTTAT |
| | | | | | | GGGTCTGGGCACTGAATTCAGTCACAAACCCTAGCATGCTCCTTT |
| | | | | | | GCTACCTCAGGGGACAAAG |
| Hsl008a01 | Chr17 | 4003001 | CCAGACCAAGTGACAGTGG | GACTGCCAGGAACGTTAGC | CCACTTTTGGACAAGTGCC | CCAGACCAAGTGACAGTGGTGACTGCCAGGAACGTTAGCCCCCT |
| | | | | | | GAAGTATCAGCGTTTGAGTTCTCTGGGCATTCTGTGGGCCCTGC |
| | | | | | | AGTGGCACTTGTCCAAAAGTGG |
| Hsl008a02 | Chr17 | 4003501 | CTGGCCATGAGTACTTTCC | CTTGTCTTGTCCCTTAAGGG | CAGCCATCACTATCTATTGC | CTGGCCATGAGTACTTTCCTCTTGTCTTGTCCCTTAAGGGTTACT |
| | | | | | | TTTTGCAGTGCAGCAAGAGAGACCGACATCAACCCTGAGTTACA |
| | | | | | | AGCAATAGATAGTGATGGCTG |
| Hsl008a03 | Chr17 | 5000001 | CTCAGGTTTTGGAATGAAGC | GACCTGCCTGGGTGAACC | CATGTGATCGCCAGAATCG | CTCAGGTTTTGGAATGAAGCTATGTCAAAAAGACCTGCCTGGGTG |
| | | | | | | AACCCCTGCAAATGGAGGTCAGCTGGACCTCAGTAAAAGCCCAG |
| | | | | | | TGGGAAGGAGCGGGGACGATTCTGGCGATCACATG |
| Hsl008a04 | Chr17 | 5000501 | CACCATGTACTCTTCACAGG | GTGGACCCAACTCTGTTGG | CTCTTACCTCTCGGATACC | CACCATGTACTCTTCACAGGCAGGTGTCTTCTGGTGGACCCAAC |
| | | | | | | TCTGTTGGTACTTGTCGTCTCCAAAAAGTCCCCAAATGCGCTAGA |
| | | | | | | GGCCAGCCAGCCCGGTATCCGAGAGGTAAGAG |
| Hsl008a05 | Chr17 | 5002001 | GTGGAGTTGATCATTTGAGG | CTTTGGCTAAGAGGGACGG | CCTTTCTTGATGATTCTCTGG | GTGGAGTTGATCATTTGAGGCCTTTGGCTAAGAGGGACGGTGGT |
| | | | | | | TATGTGCTGGGAGTGGGCAGAGGTCTGGGAGGCTTTCTGGCAG |
| | | | | | | ATTATCCAGAGAATCATCAAGAAAGG |
| Hsl008a06 | Chr17 | 5002501 | CACTAGTATGTAGAGTGTGG | CTTGAGATGGAATTCTCACC | GAACTGGGCTGGTCTTTCC | CACTAGTATGTAGAGTGTGGGAAAGCCTTGAGATGGAATTCTCAC |
| | | | | | | CTTCGAGTTCATCAGGAAATTCACACTGGAGAGGACCTTTGAATG |
| | | | | | | CCAGTGTGGGAAAGACCAGCCCAGTTC |
| Hsl008a07 | Chr17 | 47001643 | GTGTTTTGAAGCTAAGATGCG | GCCCTCCCAGAATCTTAGG | CTACTGTTTCTGTGATCAACC | GTGTTTTGAAGCTAAGATGCGTTCAGCCCTCCCAGAATCTTAGGG |
| | | | | | | ATTATATGAATCCTCTATTTAAATTCTGTTCCCAGCCCTGAGGGTT |
| | | | | | | GATCACAGAAACAGTAG |
| Hsl008a08 | Chr17 | 47002246 | GACTAATGTAAACCACCTGG | GTAAGAGAATGAGAATTCTCC | CGAGTATCCCATTTCTAAGC | GACTAATGTAAACCACCTGGTTGGTAAGAGAATGAGAATTCTCCT |
| | | | | | | GTCCTGGAGAATCAGTTCTTGGGTGTTTGGATCATCTTACTGGTG |
| | | | | | | GTCTTGCTTAGAAATGGGATACTCG |
| Hsl008a09 | Chr17 | 47002726 | CGTACTATGTCTGTTCACC | CCAACACCAACAGCGTAGG | GGAAAGTCCTTGAAAGAAGG | CGTACTATGTCTGTTCACCCACCCCAACACCAACAGCGTAGGAG |
| | | | | | | GAGATGACTTATGCCCTCCAGTGCDACTTATAAATGGTAGTTTTC |
| | | | | | | CCTTCCTTCTTTCAAGGACTTTCC |
| Hsl008a10 | Chr17 | 47003505 | GGATGGGAATGGAGTGACG | CCTGGGGAGGAGTACAGG | GGTAATCTGCTTTTCTAAGG | GGATGGGAATGGAGTGACGAGTCCCTGGGGAGGAGTACAGGTG |
| | | | | | | CTTATCTGAAAGTCAGAACTCTTGAATTCTAGACCTGCTTCTGAC |
| | | | | | | CTTAGAAAAGCAGATTACC |
| Hsl008a11 | Chr17 | 47004235 | GAGCTTTCATTTCACATGGG | CCGAAGTTGCTTTCTCTAGG | CTCCAAAAGGGTCCTGTGG | GAGCTTTCATTTCACATGGGCCCGAAGTTGCTTTCTCTAGGATCA |
| | | | | | | GCCACCCAGACTTGAATCTTCCATCCCCTTGTCTCCTTTCCCCAC |
| | | | | | | AGGACCCTTTTGGAG |
| Hsl008a12 | Chr17 | 47004713 | CCATTCATCCCGTATCAGG | GCCAAGGTACCTTTACAGG | CCACTTATCCCTAAGGAGC | CCATTCATCCCGTATCAGGGGCCAAGGTACCTTTACAGGAGCAC |
| | | | | | | CTAGAGCGAGGGCCTTTGGCAAAAACAAAACAACCAACACACCT |
| | | | | | | CTCCACAGGGCCAGCTCCTTAGGGATAAGTGG |
| Hsl008b01 | Chr18 | 8000599 | CTGGGAATAGGATCCTTAGG | CGGACATTAGTCTAAAGTGG | GTGTGAAATGGATGAGGCG | CTGGGAATAGGATCCTTAGGAATAAATATTTATGTTCACGGACAT |
| | | | | | | TAGTCTAAAGTGGCATCTTTAAACCTACCTTTTTTGTGTGTGATAG |
| | | | | | | AAACATAGAGTTACACCTTATGGTGACCGCCTCATCCATTTCACA |
| | | | | | | C |
| Hsl008b02 | Chr18 | 8001206 | CAAGTCTCTGCTGAGAAGG | CACATTTCTTTCCTGTGTCC | CACTTACAGGCCTAACTAGG | CAAGTCTCTGCTGAGAAGGGCTGGCACATTTCTTTCCTGTGTCCT |
| | | | | | | CTGTTAGGGGATAGCGATAGACTCCTCGTAAACTCCAGGATGGA |
| | | | | | | GCCTAGTTAGGCCTGTAAGTG |
| Hsl008b03 | Chr18 | 8001633 | GTGAAGTGATTCCAAGAATCC | CTGTATGGCTCCCAAAACC | CCAACTGGCTGCTAGAGC | GTGAAGTGATTCCAAGAATCCAGTAGTTAAGTCTGTATGGCTCCC |
| | | | | | | AAAACCCATGTCCCCTTCTCTGCCTAATCTTCCTTAATAAAAAGCC |
| | | | | | | AGTTGATAGTTTTTCTTTGCTGAGCTCTAGGAGCCAGTTGG |
| Hsl008b04 | Chr18 | 8002273 | GAAGCAAATGTTCAGAAGGG | GAAGGTCCTGCCATCAGG | GAGCTAGCATGCATTCAGG | GAAGCAAATGTTCAGAAGGGAATGAAGGTCCTGCCATCAGGACA |
| | | | | | | AGACATTTGGGTAGTAGAGCACATAATTCCTTACCAGGTATGATT |
| | | | | | | TGACCTGAATGCATGCTAGCTC |
| Hsl008b05 | Chr18 | 8002776 | CAGAGGTGGAGTAAAGTGG | GAACATTTCTCCGTGATTGC | CTCAAGTTGTCAAATCAGTGG | CAGAGGTGGAGTAAAGTGGATTTCACAGAACATTTCTCCGTGATT |
| | | | | | | GCAATTCTCAGGCTGAGATGGACAAGAAATGCTGATACATCTCTG |
| | | | | | | CCCACTGATTTGACAACTTGAG |
| Hsl008b06 | Chr18 | 8003814 | CTGCTCTCCTAGTGTTGCC | GGCCTTCTGTCTGTGACC | GAACTTGGTGCTTCTATGGC | CTGCTCTCCTAGTGTTGCCTCTTGGCCTTCTGTCTGTGACCATTC |
| | | | | | | TACTTAAAGAAACTTAGGGAAGAAGGAAGATAAATATTCGCTTTC |
| | | | | | | CTTTTCTTGGCCATAGAAGCACCAAGTTC |
| Hsl008b07 | Chr18 | 59000111 | GTCGATGAGTGAGGTTTCC | CATGCCATCTTCCCCTACC | GGAAATGAGTACCAACTCG | GTCGATGAGTGAGGTTTCCCTCACACATGCCATCTTCCCCTACCT |
| | | | | | | CTCCTCTTGAAAACAATGTCTTTTGCACCCTCAAGGTCAAGGTTA |
| | | | | | | AACCCGAGTTGGTACTCATTTCC |
| Hsl008b08 | Chr18 | 59001708 | CAAGGAAAGCTCTGAATTGC | GCTTGTTGTAGTTACTCTGG | CTCGGTAACGTTCTCTTTGC | CAAGGAAAGCTCTGAATTGCGCTCGCTGTTTGGTTTTTGCTTGTT |
| | | | | | | GTAGTTACTCTGGGGGAAGAGCCGGGGGCAAGGGGGTCAAATG |
| | | | | | | GGGCTAAAGTTTCAGATTTGCAAAGAGAACGTTACCGAG |
| Hsl008b09 | Chr18 | 59002384 | GAACCCTGAAGGCATAGCC | GAGTTGACCCAGCGTTTCC | GCATGTCCAACGAGACTGC | GAACCCTGAAGGCATAGCCATCTTGAGAGTTGACCCAGCGTTTC |
| | | | | | | CCTTTCATTTATTTATATAACCTGGGAAAATCTTTTCCCTTTAGTGT |
| | | | | | | CACCCTTGCAGTCTCGTTGGACATGC |
| Hsl008b10 | Chr18 | 59002822 | CTCTGGCCATTGACTTTGG | GTGACCTTTCTTTTCAGTGC | CATCTCACGACAACTGTCC | CTCTGGCCATTGACTTTGGCGTGACCTTTCTTTTCAGTGCTTCTG |
| | | | | | | ATTTTCGCTCTCTGCAGATACTCAAGTAACTGTGCCTTTCTAACA |
| | | | | | | GGACAGTTGTCGTGAGATG |
| Hsl008b11 | Chr18 | 59003252 | CCAGTTCTCACCGGAAAGG | GTTGTGACTGTAGTAAGTGC | CGATTCCAGTCTCTGAACC | CCAGTTCTCACCGGAAAGGCGTTGTGACTGTAGTAAGTGCTGAG |
| | | | | | | GGTTGAGAGGAGAGATTGAGAGTTGTTAGGGGAACTGTTACACA |
| | | | | | | GGGTTCAGAGACTGGAATCG |
| Hsl008b12 | Chr18 | 59004131 | GCATCCAGGGCTGAAACC | GCAGCTGATGCCGAGAGG | CGTGTTTACAGCAATCTTTGG | GCATCCAGGGCTGAAACCAAGGCAGCTGATGCCGAGAGGAGCC |
| | | | | | | AAAGGGCAGTTCTTCTTAGTTTAGAAACAGCAAGACAGCCTCTGC |
| | | | | | | CAAAGATTGCTGTAAACACG |
| Hsl008c01 | Chr19 | 11000001 | GCCAATGCATTTCCAAGCC | GGATCCAACCGTGGACCC | CTGCATTCGTCTTCATTCC | GCCAATGCATTTCCAAGCCCGGATCCAACCGTGGACCCTGGCCT |
| | | | | | | TTTGGGCCAGCAGAAGAGGTGGCTGTTTTTTCTCATGAAATATTT |
| | | | | | | TTGAAGGAATGAAGACGAATGCAG |
| Hsl008c02 | Chr19 | 11000501 | CTTGCCCATGGAATGAAGC | CCAATCCCCTCCCCAGGG | GTTACAGGTTAGCTTTTCAGG | CTTGCCCATGGAATGAAGCCCCCCATCCAATCCCCTCCCCAGGG |
| | | | | | | AACAGCTTTATACTAACTCTGGTGGTCGGCTTTTGGAGGGGCCAT |
| | | | | | | AAATGGCCTGAAAAGCTAACCTGTAAC |
| Hsl008c03 | Chr19 | 11002501 | GATCCAGGTGTATCTCTGC | GCTTGTAGCATACATAAGGC | CATCAGAACTATGTCTGAGC | GATCCAGGTGTATCTCTGCAAGTAGAGCTTGTAGCATACATAAGG |
| | | | | | | CCCTGCAAAGGGATTTCTGGGCCGGAAGTTTTTGATCAGTTGCT |
| | | | | | | CAGACATAGTTCTGATG |
| Hsl008c04 | Chr19 | 11003001 | GACGACATCGGAGGATCC | CAGGTTACGGCAGGAGAGG | CATCAGCAACAGATCAATGC | GACGACATCGGAGGATCCGACTCAGGTTACGGCAGGAGAGGGA |
| | | | | | | GGCCAGGCCGGGTTAGGGTTCTGGGGTTTGGGATTCTCTTCCGA |
| | | | | | | GGCTGGCATTGATCTGTTGCTGATG |
| Hsl008c05 | Chr19 | 16003501 | GCCTAACATGGCGTGTAGG | GTCAGGGTTCCAGCATGC | CATTCTGTAGAATGCTGAGC | GCCTAACATGGCGTGTAGGAGCTATGTCAGGGTTCCAGCATGCC |
| | | | | | | TTGACATGCCTCCTACACGATCCAACATGTTCCGCAACCCCTGAG |
| | | | | | | CACAGCTCAGCATTCTACAGAATG |
| Hsl008c06 | Chr19 | 16004001 | CCAGACATGAGCAAACAGC | GTTAGACAGGTGGAAGTCC | GAACACGTGACCGATGTGC | CCAGACATGAGCAAACAGCAACAGAGGTTAGACAGGTGGAAGTC |
| | | | | | | CAGGGGCGATGGAGGCAATGGCATCTCCACCACAGCCCCTCCT |
| | | | | | | GTCTGCACATCGGTCACGTGTTC |
| Hsl008c07 | Chr19 | 45002590 | CTGTCTTTCCACCAAACTGG | CCATCAGGCTGTGATCAGG | GTGTTTGGTTGGGAAACAGG | CTGTCTTTCCACCAAACTGGGCACCATCAGGCTGTGATCAGGGT |
| | | | | | | TCCAATCACACAAAGACCCCAGCACCCTCTGTCTAAAACTCATCT |
| | | | | | | CCGGCCTCCTGTTTCCCAACCAAACAC |
| Hsl008c08 | Chr19 | 48000001 | GTTCTAGGGCTGACAGACC | CAGCAGACAGTGGAAACGG | GTATGTGTCTTCAAACTGCC | GTTCTAGGGCTGACAGACCGAGACTGTGGCAGCAGACAGTGGA |
| | | | | | | AACGGTGGCAAAAAGGGGGCAGATGAGGAGGAAGGGGAGAGAA |
| | | | | | | CACAACCTAAATCCGGCAGTTTGAAGACACATAC |
| Hsl008c09 | Chr19 | 43002501 | GATGCCCAGCTGCTGAGG | GCAACTGGTCAGTCTAAGG | CCTTTGTCGTGATCTGACG | GATGCCCAGCTGCTGAGGAGCAACTGGTCAGTCTAAGGACAGAG |
| | | | | | | AAGAGCTACTGGTCAACACAAATTCATCCTCATCTGGGAACTAAC |
| | | | | | | ACGTCAGATCACGACAAAGG |
| Hsl008c10 | Chr19 | 58000001 | CAGTTCCTCATGTACAGTCC | GGACAAAAGGAAACGTCAGC | GTTGATCATCCCTCCTGTGC | CAGTTCCTCATGTACAGTCCGTATGGACAAAAGGAAACGTCAGC |
| | | | | | | CAGGCTGCTGGAGCAGCCCCTTTGGTGCCTAAGTTTCCCTAGCC |
| | | | | | | GTCAGCACAGGAGGGATGATCAAC |
| Hsl008c11 | Chr19 | 58003501 | CCTTCATGCCTGCTTGGG | GTTGTGACTTCAGCCATACC | CTACTGGTATGATATGAATCC | CCTTCATGCCTGCTTGGGAAGTTGTGACTTCAGCCATACCGAGA |
| | | | | | | GATAGTTGGTGGGTGGAGCTCAGGGAGGTGTGAACTCAGGGAT |
| | | | | | | GGATTCATATCATACCAGTAG |
| Hsl008c12 | Chr19 | 56308828 | CAGGCATTGTATGAAGTTCC | GGATACAGCAGAAAACTGG | GGCACATGATACATTCAGC | CAGGCATTGTATGAAGTTCCTGGGATACAGCAGAAAACTGGAAG |
| | | | | | | AAATACGATGGAATTCTAGCATTGTAAAGACAGGGCTGAATGTAT |
| | | | | | | CATGTGCC |
| Hsl008d01 | Chr2 | 30000161 | CATGACCTTCTTAGAGACC | GGTCTCTTGAAATCATCACC | CTTCTTCCCTACAAACTAGC | CATGACCTTCTTAGAGACCAGGGTCTCTTGAAATCATCACCCAGC |
| | | | | | | CTACGAGTCACTGGCTGAGGTCACCTGACAGTGAGTCACTGGCA |
| | | | | | | GCTAGTTTGTAGGGAAGAAG |
| Hsl008d02 | Chr2 | 30002357 | CTGAGTCCGAATTCAAGCC | CTCTTCACCAGCAATACGG | GGTGACTTCTCTAAACATCC | CTGAGTCCGAATTCAAGCCAAGGCTCTCTCTTCACCAGCAATACG |
| | | | | | | GACTCCTGAATAGAGTCTGCATCATTCTCTCTGCAGAATGCTCGG |
| | | | | | | TGGATGTTTAGAGAAGTCACC |
| Hsl008d03 | Chr2 | 30002837 | GGCTTTGGGACAAGATTCC | CTTGGGAATGCTGAGAACC | GAGAGCACCTGTAGAGATCC | GGCTTTGGGACAAGATTCCTTGATCTTGGGAATGCTGAGAACCA |
| | | | | | | AATAACCAGCATCATTGTGGACCAAGCATCCCAGCCCCAAACAC |
| | | | | | | AGTGAGTATTGACTCTGGATCTCTACAGGTGCTCTC |
| Hsl008d04 | Chr2 | 30003549 | CGTTAGCACAACCCATGGC | GGGTAACAGATGCCACAGC | CTTCATCAACTGAAAAGATGC | CGTTAGCACAACCCATGGCGTTTCGGGGTAACAGATGCCACAGC |
| | | | | | | AAAAAGCCCATGCTGGTTAAGGAAGATGATACTGGCGAGAGTGT |
| | | | | | | CTCCAAATCTTTCTAGCATCTTTTCAGTTGATGAAG |
| Hsl008d05 | Chr2 | 30004277 | CCCATCCTCCTTGCATGG | CCTTGCATGTCACCAAAAGG | GTGTGCCTATTGCATTGGC | CCCATCCTCCTTGCATGGGCCTTCCATGTCACCAAAAGGCTCCC |
| | | | | | | CACCTCCAGGAAGGAGAGAGAACATGCCTGCAATCACACAGCCA |
| | | | | | | ATGCAATAGGCACAC |
| Hsl008d06 | Chr2 | 30004655 | GGCTGTCTTCTTTGTCTCC | GTCCTCTGCTAACCTGTCC | CAGTTCTTTCTGTCTAGAGG | GGCTGTCTTCTTTGTCTCCTGTCCTCTGCTAACCTGTCCTACGAC |
| | | | | | | ACAAAATAAACCTTCTCACAGCTTTTGGGTGTATGAACTGCCCAC |
| | | | | | | AGGGAGTTTCCTCTAGACAGAAAGAACTG |
| Hsl008d07 | Chr2 | 205000501 | GCAGTTAGGGAAGGTTCCC | CTGCTAGTCTGAAGACTCC | CAGTGAAACAGAGCAGTGC | GCAGTTAGGGAAGGTTCCCAGAGGCTGCTAGTCTGAAGACTCCT |
| | | | | | | GGGACCTCCTGATGTCTTTTAAGCCCACACATTGTGGCCCAGTG |
| | | | | | | ACTGATTTAGCACTGCTCTGTTTCACTG |
| Hsl008d08 | Chr2 | 205001001 | CAAGCCACAAACTGTAGGG | GTCGCAACAATACCACAAGG | CTGACTCCTGAACAATGTCC | CAAGCCACAAACTGTAGGGCAGTCGCAACAATACCACAAGGATA |
| | | | | | | AACTTAGGGCAAAATTCAGAAAAGAAATTGTGGTAACAGACTTGG |
| | | | | | | GACATTGTTCAGGAGTCAG |
| Hsl008d09 | Chr2 | 205002001 | GTGTGATTACTCACTAATCCC | CTCTCACTTTTGACCAGACC | CTTGAGTGGCTTTCCAACC | GTGTGATTACTCACTAATCCCTTTCCCCTCTCACTTTTGACCAGA |
| | | | | | | CCCATATGTTGAACTCCAGAATGACTTGTGGATGGAGGCTTGAAC |
| | | | | | | TTGGAGCATTTGGTTGGAAAGCCACTCAAG |
| Hsl008d10 | Chr2 | 163291858 | CTGTCATTGTAACGTTTCCC | CTGTCCTAAGGAATCCAACC | GATTGCTCACTGGCTGGCTTG | CTGTCATTGTAACGTTTCCCAATTTGCTGTCCTAAGGAATCCAAC |
| | | | | | C | CATCCGATTTTGTCAGTCAGGTAAGGCCTTTCTACATTCCCAATC |
| | | | | | | GCATACCAAATGCAAGCCAGCCAGTGAGCAATC |
| Hsl008d11 | Chr2 | 236000001 | CTTGTGACTTACCCTTACGC | CTTTCTGTCTCATCTGAAGG | CTAGGAGAAGACATCCCTCG | CTTGTGACTTACCCTTACGCAACCTGGTGGGCACCCACTTTCTGT |
| | | | | | | CTCATCTGAAGGCTGACTGGCTCTGCCCCTCACAGGCGGGGGC |
| | | | | | | CAAGGACACCAGATCTCCCGAGGGATGTCTTCTCCTAG |
| Hsl008d12 | Chr2 | 238335461 | CAGGTTAGTAGTACCATGGC | GCTGTGTACTGCAAAGATGG | GCAAGCCTGAATGTATTTTGG | CAGGTTAGTAGTACCATGGCAACAGCTGTGTACTGCAAAGATGG |
| | | | | | G | TGGAAGATAGTTTCCTAAAACATAAGGATCTTCTCTTTCCACATCC |
| | | | | | | TCTCTTTTCCCAAAATACATTCAGGCTTGC |
| Hsl008e01 | Chr20 | 21000366 | GTTCCGTCCGATTCTTCCC | GTGCTCAAGCCACAATACC | CATCTTGGAGATATCTACCC | GTTCCGTCCGATTCTTCCCTCATATTGTGCGTGCTCAAGCCACAA |
| | | | | | | TACCTAGAATCCTGAGCATTGTAAGTGTTTAGTAAACACCTGCTT |
| | | | | | | CCAAACAGTGGGTAGATATCTCCAAGATG |
| Hsl008e02 | Chr20 | 21000532 | CTGATTCTATGGGCAGCGC | GCGTTTGTTTGCTTGAAAGC | CGGAATTCAACATTCCAAGC | CTGATTCTATGGGCAGCGCCTGGCGTTTGTTTGCTTGAAAGCCC |
| | | | | | | TGACTGATGGGGTTAGACAATTATGACCTTGGTTCCTAAAGAGCA |
| | | | | | | AAGTGCTTGCTTGGAATGTTCAATTCCG |
| Hsl008e03 | Chr20 | 21001220 | CTCCAATACTGCACAATCCG | CACTCATTTGCTCCGTTGC | GTGCTTAGAGTTGCCTGGC | CTCCAATACTGCACAATCCGCCCTCACTCATTTGCTCCGTTGCCT |
| | | | | | | GTCGAAAGCACAGAGCGTAATTACTAAAGTTAAGAAAACATCCCT |
| | | | | | | GTAATTAGCCAGGCAACTCTAAGCAC |
| Hsl008e04 | Chr20 | 21001521 | CTTTCGTAGACAGCAGCC | CTGGGGAAACAGACACAAGC | CCCTAGGTTAACAGATGCC | CTTTCGTAGACAGCAGCCAGAATAAAGTCTAATATTCCGGCTGGG |
| | | | | | | GAAACAGACACAAGCAAACAGTGANNNNNNNNNNCCTGTTGATT |
| | | | | | | TTATTTTCCTTTGTGGGCATCTGTTAACCTAGGG |
| Hsl008e05 | Chr20 | 21002551 | CAGCTCCACAACTAGTAGG | GGGAAATGTAAAGTCTGAGG | CCTTGTCCAAAACTTGAACG | CAGCTCCACAACTAGTAGGTACATTGACTCAACATAGAGAAAACG |
| | | | | | | GGAAATGTAAAGTCTGAGGGTTGTGTGTTTGGGAGAGGTGGGGT |
| | | | | | | GGGGGTGTCTCATTTTTAAAATACACGTTCAAGTTTTGGACAAGG |
| Hsl008e06 | Chr20 | 21003101 | CAACCACATTGATGTGAGC | CATACATCTTCAGCCAAGGC | CTCACCTGGCATTAGATCC | CAACCACATTGATGTGAGCTCCTCATACATCTTCAGCCAAGGCAC |
| | | | | | | ACAGAAAAAGGAAATGCCTGACAAACAACCCTTCTGAGTGAAGAA |
| | | | | | | TGATGGGATCTAATGCCAGGTGAG |
| Hsl008e07 | Chr20 | 58000746 | CTGCAGCACCTGTCATGG | CTCTGTGTCACGTAGTAGC | CTTGACAATCCACTGTTTCC | CTGCAGCACCTGTCATGGGGGACTCGTGCTCTGTGTCACGTAGT |
| | | | | | | AGCTGCTCAATAATTCCTCCTGGAGGGGATTGCTGATGGAGTCC |
| | | | | | | TTGCTTTTCCCTGGAAACAGTGGATTGTCAAG |
| Hsl008e08 | Chr20 | 58001127 | CTTTGCTCAGACCAACACG | CTGAGTTGCCATGCATTCG | GGTACCCAGGCATATCTGG | CTTTGCTCAGACCAACACGTCTGAGTTGCCATGCATTCGAAGAGT |
| | | | | | | GGGTGCCATGGTTCCCAGCAGACATGTGGCTCAGGACTTGGCCA |
| | | | | | | GATATGCCTGGGTACC |
| Hsl008e09 | Chr20 | 58001573 | CAGCTCAGGATGGAAAAGG | GAGCTAGGAGAGGTACAGG | GAGGTTGAGTAACATGTTCC | CAGCTCAGGATGGAAAAGGCAAATTGGGAGCGGGGCCAGAGCT |
| | | | | | | AGGAGAGGTACAGGATGGAGAGAAGTTGCTGGGAAGGGAGGCC |
| | | | | | | GAGGTAAGGCCGGGCCGGTGAAAATGGGAACATGTTACTCAACC |
| | | | | | | TC |
| Hsl008e10 | Chr20 | 58002128 | CAGAGCAAGAGGGATGGG | CTGGTGCTGAGACTCTGG | GAAGCACAGTTTAGAAATGGC | CAGAGCAAGAGGGATGGGACTGAGTCCTGGTGCTGAGACTCTG |
| | | | | | | GGGAGGGACAGACTACTTTGTGATTACTCAAAAGGCGAGGAGGG |
| | | | | | | GTGATGAAATAAGCCATTTCTAAACTGTGCTTC |
| Hsl008e11 | Chr20 | 58002880 | GAGGGACCAAACTATGAAGG | CTGATGAGCCTTAGAATTGG | GAAAGGGCTCCTATAGATGC | GAGGGACCAAACTATGAAGGAATGCTGATGAGCCTTAGAATTGG |
| | | | | | | TTTCTGTCTGCGTCCCACTTCTTGCAAACCCCTAGTTATTAAACAC |
| | | | | | | TGTAATTCTTGCATCTATAGGAGCCCTTTC |
| Hsl008e12 | Chr20 | 58004256 | GAAGTGTCAACAGCATAGCC | GGAAGATTCTGGAGATACC | CTTCCACCATAACATTTGGC | GAAGTGTCAACAGCATAGCCCAGGAAGATTCTGGAGATACCTAA |
| | | | | | | ATTAAAGCAGCATGAGTGTAGGGGAGCCCCTGTTTCTCAAAGCC |
| | | | | | | GGGGGTGCCAAATGTTATGGTGGAAG |
| Hsl008f01 | Chr21 | 38001523 | GCATCCACACGTGATGTGC | CAAGCTTCTGAAGCTACGC | CTTAGGATGGAAACCATCGC | GCATCCACACGTGATGTGCGTCAAGCTTCTGAAGCTACGCTCCT |
| | | | | | | GAGGAAGGCTTTGTGCAGCTCAGACTTCCCCACCATCTGCTAAC |
| | | | | | | CATGCCCTGCGATGGTTTCCATCCTAAG |
| Hsl008f02 | Chr21 | 38002847 | CTAACCTATTGCCAGCTGC | GGTTACAATTCATCCCACCC | GACCATCTAACATCACAAGG | CTAACCTATTGCCAGCTGCACACAGGAGTTAGAAAAAGGTTACAA |
| | | | | | | TTCATCCCACCCGATTTGAGATTTTTCCAGTTAAAGACATGGCGA |
| | | | | | | GGTAGAAAGACCAAGTCCCTTGTGATGTTAGATGGTC |
| Hsl008f03 | Chr21 | 38003592 | GGACTGCAGCTAGTATGGC | CCCATAGCTATTGAAATGCC | GGATGGCTGTTGTTCATCC | GGACTGCAGCTAGTATGGCCCCCATAGCTATTGAAATGCCCGAG |
| | | | | | | ACACGTCAGTGTCTAAACATCTCTCAGACCACCCCTTCCACTTGG |
| | | | | | | ATGAACAACAGCCATCC |
| Hsl008f04 | Chr21 | 40002740 | CTTTGTTAAGCTCACTTTGC | GGAATTCAGAGCTCATAGGG | GTAGTGCTTCTCAGTTTAGC | CTTTGTTAAGCTCACTTTGCAACATAAGAGGAATTCAGACCTCAT |
| | | | | | | AGGGATGTGAGCTACATAGTTATTCACCATATACCCTCAGGAAGA |
| | | | | | | AGTAGAGCTAAACTGAGAAGCACTAC |
| Hsl008f05 | Chr21 | 40003005 | CAGGATGTGACCACTGGC | CATTCCTAATGTTTCAGGTGG | GTTGAAGGAATTGGAAGAGG | CAGGATGTGACCACTGGCTCATTCCTAATGTTTCAGGTGGGTAAC |
| | | | | | | AAATATTTGCTTTTTCCGGGGAGTTGACCACACACCCTCTTCCAA |
| | | | | | | TTCCTTCAAC |
| Hsl008f06 | Chr21 | 40003784 | CCACAGACAGTTCTAGAGG | GTGCTTGACTTTGGAAACCC | CTTGAGGAACGAGTTTCTGG | CCACAGACAGTTCTAGAGGGTGTGCTTGACTTTGGAAACCCAGT |
| | | | | | | TTAACTGGCTTCTGCTTGAATCATCACTCCATTAACATCATCATTC |
| | | | | | | CAGAAAGTCGTTGCTCAAG |
| Hsl008f07 | Chr21 | 40302108 | GAAGTTTCTGGGACACAAAG | GCTTTCTGGCTTTGTCAAGC | GGAAAACTTGGTAAAAGTGAC | GAAGTTTCTGGGACACAAAGGGCTTTCTGGCTTTGTCAAGCTGG |
| | | | | | | TCTTGAGAGATGAAACAGGCACCCCGCGCCATGTGCTAACAGTC |
| | | | | | | ACTTTTACCAAGTTTTCC |
| Hsl008f08 | Chr21 | 40303890 | GTCATTGCTGGAAATTGATTC | GAGTTTCAGAGCTTCTCTAG | GTGTCAGGATCCCTGAATC | GTCATTGCTGGAAATTGATTCATAGAGTTTCAGAGCTTCTCTAGA |
| | | | | | | AGGCCTCAGCCATGTCCTTAAAAGTTGCATAAAACTTTTGGCATA |
| | | | | | | TGAGTGATTCAGGGATCCTGACAC |
| Hsl008f09 | Chr21 | 40304216 | CTTCTTCTCTTCAAGGGTAG | CATGGTGGACGTGGATGC | GGACTCAGCACTCACAATG | CTTCTTCTCTTCAAGGGTAGACATGGTGGACGTGGATGCAGGAT |
| | | | | | | AGCAGGCATCAGGCAGATGTGAATGGCATGGAAAACCAGGCTCC |
| | | | | | | TGGAGACATTGTGAGTGCTGAGTCC |
| Hsl008f10 | Chr21 | 40304979 | CGTCAACACGGATTACATTC | GAAACCATGGATGCACACC | GATTCAGTGACACAGAATGG | CGTCAACACGGATTACATTCTGAAACCATGGATGCACACCTCACA |
| | | | | | | TTCCTGGAGTCATCTAACACTAGCATCAGCAGGTGGTCTTGACAT |
| | | | | | | GGTCCTGGACCCATTCTGTGTCACTGAATC |
| Hsl008f11 | Chr21 | 44002659 | CACCAGCCAGCATTCAGC | GCTTTGAGGTGGCGATCG | CTTCCTTTGTGAGTTGTGG | CACCAGCCAGCATTCAGCACAGCAGCTTTGAGGTGGCGATCGCT |
| | | | | | | ATTTCCCCAACTCAATGAACTAAAGTACTAGAAGAAAATCTCCCA |
| | | | | | | CAACTCACAAAGGAAG |
| Hsl008f12 | Chr21 | 44003281 | CTCCAGCCTGTCTGTAGG | CTACTCCTGGAAGCTCACC | CTGAGACGCACAGTATAGC | CTCCAGCCTGTCTGTAGGTAGGAAAAACTACTCCTGGAAGCTCA |
| | | | | | | CCTCAGTGAATGCACCTCAGAGTCCAAGAGCTGCCGCGAATACA |
| | | | | | | GGGCCTGGTGGCTGCTATACTGTGCGTCTCAG |
| Hsl008g01 | Chr22 | 20000269 | GTAAGCCCTGTGGTTCTGG | CGGTATCCATGGTCCAACC | CTGTAGCTTGCCAATCTGG | GTAAGCCCTGTGGTTCTGGCACGGTATCCATGGTCCAACCAGAG |
| | | | | | | GGCTGAGAGGTCTCACACTGGGGCATAAGCCTGGCCCAGGCCA |
| | | | | | | CACAGCCAGATTGGCAAGCTACAG |
| Hsl008g02 | Chr22 | 20000673 | GCTCAATGACAATGCTGTCC | GCAAAACCGAGTGTTCTCC | GGTCTGTGCCTCAATGTCC | GCTCAATGACAATGCTGTCCACTACAGCAAAACCGAGTGTTCTCC |
| | | | | | | TAGGCCTGCTGCCACCCTGGGCACATAGTGAGAACACGCCCACT |
| | | | | | | TCTGCTGTGGACATTGAGGCACAGACC |
| Hsl008g03 | Chr22 | 20001252 | GCTCTGGGTCATCTTCCC | CAGGCCAAGATATGAAGGC | GTCTTGGGTCACTCTGAGG | GCTCTGGGTCATCTTCCCGACCTGAAACAGGCCAAGATATGAAG |
| | | | | | | GCCCTGAGCCAGGAAAACCTACTAAGGGATCCGTGATCCCAAGT |
| | | | | | | CCCCTCAGAGTGACCCAAGAC |
| Hsl008g04 | Chr22 | 20002510 | CACCTCTGGAGGGAGTGC | GCAAACATGGGAGCCAAGC | GGGAGAACAAGTTCTGACC | CACCTCTGGAGGGAGTGCCAGAGCAAACATGGGAGCCAAGCAG |
| | | | | | | CCCAGATGTGGTGGGTGGGGAGACTCAAATTTAGCAGGATTGAG |
| | | | | | | GGTCAGAACTTGTTCTCCC |
| Hsl008g05 | Chr22 | 20003050 | GACCAGACCTCTAAACACC | CCAGATCCCAGAGTAAAGG | CACCTCTCCCGACCTTAGC | GACCAGACCTCTAAACACCGCCCAGATCCCAGAGTAAAGGCAGA |
| | | | | | | TAAGGCAGTAGTTAAGAAGTAGGAAGAAGTAAAGGCAGCTACCC |
| | | | | | | CAGAGAAGCTAAGGTCGGGAGAGGTG |
| Hsl008g06 | Chr22 | 36001711 | GCATACGAATTCCCAAATCC | GCAGAAAGGAAGAAGGTTCC | GTGGACACGTCCCAAATCC | GCATACGAATTCCCAAATCCTGGCGCAGAAAGGAAGAAGGTTCC |
| | | | | | | CCTTTAATGCGGTTGTCTGGTGCCACCGCACAGCCTGGTAAATA |
| | | | | | | AGTGTGCTAGGATTTGGGACGTGTCCAC |
| Hsl008g07 | Chr22 | 48000357 | GTTTGGAGGGATGGAAATGG | CTGGAGAAACTAGGAAGGC | CACATGGGTTACTCTTAGGG | GTTTGGAGGGATGGAAATGGAGCAGGAGGAGGCTGGAGAAACT |
| | | | | | | AGGAAGGCCCAGACCACACAGGCCTGCTGAGACACATTGCGGA |
| | | | | | | GTTTTGGCCTTTTCCCTAAGAGTAACCCATGTG |
| Hsl008g08 | Chr22 | 48000513 | CTGTGAGGATGATGGACAGG | CAGGGGACACGCATTAGC | CTCTGTTCGTGTGCTTCCC | CTGTGAGGATGATGGACAGGAGGGCAGCAGGGGACACGCATTA |
| | | | | | | GCTCCCCTGTCATCCCTCTGCCAGCACCTCCCAAGAGCAGTTTG |
| | | | | | | TGCTAGGTGTGGGAAGCACAGGAACAGAG |
| Hsl008g09 | Chr22 | 48001021 | CAGTCATCTTCCAAGTTGC | GTGGACGGATTCAATGATCC | CCTTCCACAAACTCTGTGC | CAGTCATCTTCCAAGTTGCACGTGGACGGATTCAATGATCCCAG |
| | | | | | | CTATCCCCTCCCGAAATTAAACTGATGAGCAAATGAAATGCAAGC |
| | | | | | | ACAGAGTTTGTGGAAGG |
| Hsl008g10 | Chr22 | 48001872 | GAGAGCAGAGGGCTTCTGG | GAGGACACTCCCATTCTGG | CAGCATTACAGCCCTCCC | GAGAGCAGAGGGCTTCTGGTAAATGAGGACACTCCCATTCTGGC |
| | | | | | | AGTGTCAGGAGAGTGGTTTCGGGGAGCGTGGGTAGTAGGGAGA |
| | | | | | | GCAGTGGGGAGGGCTGTAATGCTG |
| Hsl008g11 | Chr22 | 48002641 | CAGTGGATTAGCCTAAACGC | CTGAATGAGGCCACTTTTCC | CTCTGTTCTCTTTGCAGTGC | CAGTGGATTAGCCTAAACGCGGTCTGCAGCCACTATTCAGACTG |
| | | | | | | AATGAGGCCACTTTTCCCCCCAGAAGGATGTGTGTGCATGGGGT |
| | | | | | | CACAGTCCTGCGAGGGAGACCTGGCCGCACTGCAAAGAGAACA |
| | | | | | | GAG |
| Hsl008g12 | Chr22 | 48003056 | CTTCATGCTCTCATCAAACC | GTAACTTCCTGGTTCTTGCC | CAGTTCTCTGATTGAGATGG | CTTCATGCTCTCATCAAACCGGTAACTTCCTGGTTCTTGCCATGC |
| | | | | | | AGCCTACAAATGTGTCTCCAGCCCTCGCTGCTGTGTGGGTTTGC |
| | | | | | | CATCTCAATCAGAGAACTG |
| Hsl008h01 | Chr3 | 134517897 | GTTGACAAGTAGTGGGTTCC | CATTGCTGATGCATGAGTGC | GTACAGTGAAATTCAGTGC | GTTGACAAGTAGTGGGTTCCCAGTAGGCATTGCTGATGCATGAG |
| | | | | | | TGCACGACTAAATTACTGTGCCCCTTTGTGGCGTGCCCCAACGT |
| | | | | | | GAAATGCTAGGGCACTGAATTTCACTGTAC |
| Hsl008h02 | Chr3 | 44000641 | GTGAATGACATGGGTGAGG | GCATGGTGAATGCAGAACG | GCTTGTTTCTACCTGTAGC | GTGAATGACATGGGTGAGGGGTGCAGGGCATGGTGAATGCAGA |
| | | | | | | ACGGTGCCAGGTAAAGGAAGCGCCTGCTGTGCCATGCAGGTAA |
| | | | | | | GTTTAGCTACAGGTAGAAAGAAGC |
| Hsl008h03 | Chr3 | 44001242 | CACAGACAGCTGCTCAGG | CAACTGTGTAAACCTTTGCC | CTGGATCCTCCACTTGTGC | CACAGACAGCTGCTCAGGGAGCCCCAACTGTGTAAACCTTTGCC |
| | | | | | | AGTGGATTCTGAGGAGAACCCCGATATCAAGCAGATTAACGCCG |
| | | | | | | GGCTACTTTGGTTAAAGCACAAGTGGAGGATCCAG |
| Hsl008h04 | Chr3 | 44001682 | GAGCAAAGCTAATCCATTCC | CACATAGCAGCACAGAAGC | GTAGTCCTTGGAAAAGTAGC | GAGCAAAGCTAATCCATTCCCAGGTGGCACATAGCAGCACAGAA |
| | | | | | | GCCATCTGCTGCTTGCATCCACCCTGGGGGCCTCACCTGCTCAC |
| | | | | | | CACAGCTACTTTTCCAAGGACTAC |
| Hsl008h05 | Chr3 | 44002894 | GCGCTTGTCTCTTTTCTGG | CTGTGTTCTGCACATACTGC | GAATGGGCTGAATGAAGGC | GCGCTTGTCTCTTTTCTGGTCAATTCCTGTGTTCTGCACATACTG |
| | | | | | | CAGAAGATTTTCTGCCCACTGGGAAGGCTCTGTGTCTATGTTGG |
| | | | | | | CCTTCATTCAGCCCATTC |
| Hsl008h06 | Chr3 | 44003138 | GTTGGTCCTCCATAGAAGC | CCACTTGTCTGGTATTCACC | GATGAGTTCTGTGCCTTCC | GTTGGTCCTCCATAGAAGCCAAATAATCCACTTGTCTGGTATTCA |
| | | | | | | CCACTGCCCAGGAAAAGAAATGAGTGAAAGAGGCACCTGGTGAG |
| | | | | | | GTCCATTGCAGGGAGGCAGGAAGGCACAGAACTCATC |
| Hsl008h07 | Chr3 | 123000854 | GTATCCCCTTCACTTCTGG | CCTACTCAGCTCTTGTTCC | GAACACTGGTGACCATAGC | GTATCCCCTTCACTTCTGGTCCCTACTCAGCTCTTGTTCCTCAGC |
| | | | | | | CTCCTGCTCTGGGTGCACCTGCTCCCCAGCCCCATGCACTGACT |
| | | | | | | CTTGCTATGGTCACCAGTGTTC |
| Hsl008h08 | Chr3 | 123001360 | GAACATGGGATGAACTCAGC | CACACTTTACTCAGGTTGG | GCTGTTGTTCTCAAGTTCCC | GAACATGGGATGAACTCAGCACACACACTTTACTCAGGTTGGAA |
| | | | | | | GCAGAACGAAAACCCAACACCACTGGCGGGCGATGTGGAGGGG |
| | | | | | | CAGGGAACTTGAGAACAACAGC |
| Hsl008h09 | Chr3 | 123001728 | GAGGTGAAGATCATTCTAACC | GAAGCAATGGAAAGATTTGGG | CCTATGAGGAAGACATTTGG | GAGGTGAAGATCATTCTAACCTGAGAAGCAATGGAAAGATTTGG |
| | | | | | | GCATGGGCCCTAAGGATTCCACTGAATTCTGTGCTAGAGTATCAT |
| | | | | | | TTTCCAAATGTCTTCCTCATAGG |
| Hsl008h10 | Chr3 | 171001001 | CCAAAACCATTCACTTAGGG | CTTTGGTGCTAAAGCTTCC | CCCTTAACTGGCAGTCAGC | CCAAAACCATTCACTTAGGGGAATTTCAAACTTTGGTGCTAAAGC |
| | | | | | | TTCCAAATAATCAGCATCACCATTCACCAAGGAGCAGAGGAGTTC |
| | | | | | | GGTCTTGCTGACTGCCAGTTAAGGG |
| Hsl008h11 | Chr3 | 171001501 | GCCATCTTCCAGGTTTTCC | CCCACAAAGGTCTTTCAGG | GAGATCCCATTGTCTTTGCC | GCCATCTTCCAGGTTTTCCACAACCCACAAAGGTCTTTCAGGTGG |
| | | | | | | GTATAATTTGGGGTTACTTGTTAAGATGGAGTTACAGCACAGCTT |
| | | | | | | CATTGGCAAAGACAATGGGATCTC |
| Hsl008h12 | Chr3 | 196515646 | GAAGAGCCTGTTTCAGTGG | CTACAGGAGGGATCAGAGC | GTAGTGCCTACATACACCC | GAAGAGCCTGTTTCAGTGGCCCACCTACAGGAGGGATCAGAGCA |
| | | | | | | CATCCATGGAGCTGAGTGCCGCCCGGTGTTACTGGAAAGCAGAG |
| | | | | | | AGGGAAGGACAGAGAATTACAGCAGGGTGTATGTAGGCACTAC |
| Hsl009a01 | Chr4 | 25002345 | GTAGAAGAAAGATCCACCCC | CTCCACTGGGGACGGTCC | CTGCAAGAAGCATTCTTCC | GTAGAAGAAAGATCCACCCCCTCCTTCAAGCTGATCTCCACTGG |
| | | | | | | GGACGGTCCACATATTTCTCTGCTTTGCATTTTTGCTGTTGCTTG |
| | | | | | | GTTGGTTTTTTGTTTTACATTATTACTGGAAGAATGCTTCTTGCAG |
| Hsl009a02 | Chr4 | 25002882 | CAGCTGGGAATGTGATACC | GCAACACTGTGAAAAGATGC | CAGTCCCAGCAACTATGCC | CAGCTGGGAATGTGATACCTCTCTGCAGTAGCAACACTGTGAAA |
| | | | | | | AGATGCCACCTTGCCATCTCTACAGGTGGCTGGAATTGGGAACA |
| | | | | | | TCACTTTGATCTGGCATAGTTGCTGGGACTG |
| Hsl009a03 | Chr4 | 25003068 | CAACAGAAAGAATAGCTTGCC | CTTTAGGACTGGAGGAATGG | CTCTTGGTTTCTTTGAACAGG | CAACAGAAAGAATAGCTTGCCATCTTTAGGACTGGAGGAATGGC |
| | | | | | | AAAGCTCTTTCCCTTTCAGCCTCCAATGGGGGGACCTGGGCATT |
| | | | | | | TGTAGCCTGTTCAAAGAAACCAAGAG |
| Hsl009a04 | Chr4 | 25003539 | GTGCTACTTTCATGGCTAGG | GTTTCTCTTTCAGAGCTACC | GATTCTTAGTGGATGTTCCG | GTGCTACTTTCATGGCTAGGAATGAAGTTGTTGGGTTTCTCTTTC |
| | | | | | | AGAGCTACCCCTAAAGGCATTCACTTTATATTCTCTGAAGAGAAC |
| | | | | | | CAGCTAACCAGGCGGAACATCCACTAAGAATC |
| Hsl009a05 | Chr4 | 25004256 | GTTACCCACAAACTCAACGG | GTTCTGAGAAGCAGATGAGC | CTCTTTCTTACGGTTCAAAGG | GTTACCCACAAACTCAACGGGTGGGTTCTGAGAAGCAGATGAGC |
| | | | | | | CATGAAGAGCATCAAACAAGCATTACTGCTCTGGCCACCACCAG |
| | | | | | | GGTCACCTTTGAACCGTAAGAAAGAG |
| Hsl009a06 | Chr4 | 25004641 | CTTTTCAGCAGACTTTTGGC | GCTAAAGTGGAATGAGAGG | GATGAGGAAGAAGACAATTGG | CTTTTCAGCAGACTTTTGGCTTTAAGAGTTCTTACCAAAAAGATTG |
| | | | | | | CTAAAGTGGAATGAGAGGGGCTCAAAGNNNNNNNNNATTTCATT |
| | | | | | | ATAAGTGCTGTCCCATCTTATCCAATTGTCTTCTTCCTCATC |
| Hsl009a07 | Chr4 | 154000870 | GTCATGACAACTTCTGTCC | CGCTTACCGGAAACAAACC | CACTTCCCAGGCCAAGGG | GTCATGACAACTTCTGTCCCCTTCACACAGACCGCTTACCGGAAA |
| | | | | | | CAAACCTTGAACTCCCCCTGCTTAAGACTGAAGCCTCTGTCCATC |
| | | | | | | TGACCTTCCCTTGGCCTGGGAAGTG |
| Hsl009a08 | Chr4 | 154001138 | CCCAACAGGGACATGTCC | GTTACATCATGTCAGATGGC | CATCCAGATAAGCAGATTGC | CCCAACAGGGACATGTCCGTCACGCTGGTGTGTTACATCATGTC |
| | | | | | | AGATGGCAATTGAATGCGCTGTTAACATAAGCTGACAGGAAGGC |
| | | | | | | TCTAGCAATCTGCTTATCTGGATG |
| Hsl009a09 | Chr4 | 154001718 | CCAGTTTCATAGACATCTTGC | GCAAAAGATTCTTTCCTTTGC | CAAGCTGGGGATGTTTTGG | CCAGTTTCATAGACATCTTGCAAAGGAAAAGATTCTTTCCTTTGCA |
| | | | | | | AGATACTATGAAAAGTATTCATAGGAGAAGGCATCTGCACCAATC |
| | | | | | | CATAGACCCTCCAAAACATCCCCAGCTTG |
| Hsl009a10 | Chr4 | 154002285 | GGGATGTGTTGCACAAAAGC | CAGTCTGTCAACTCTTTAGG | CAACACTCTGACTTTCTAGC | GGGATGTGTTGCACAAAAGCAGGGCTCAGTCTGTCAACTCTTTA |
| | | | | | | GGTTCTGAGGGGGCCAGATGCTCCCCGTTGTTATTCCAGGCCCG |
| | | | | | | GCGGCTAGAAAGTCAGAGTGTTG |
| Hsl009al1 | Chr4 | 154002860 | GGTTCTCCTGACCTCTCC | CCAGTTGTGTTTCTGTTCCC | GTTCATCAATTGTACTCAGC | GGTTCTCCTGACCTCTCCTCCAGTTGTGTTTCTGTTCCCAAGGTG |
| | | | | | | GTGCCTCGGTGCTAATACCTCGTAATTTTTCTGTCAAACCTTTCC |
| | | | | | | AGTGGCTGAGTACAATTGATGAAC |
| Hsl009a12 | Chr4 | 154003669 | CAGCTCTACCAACCACAGC | GAAGTGGTAAAGTTTCTTCGC | CACACCCAATGAATGAACGC | CAGCTCTACCAACCACAGCAGGAGACAGAAGTGGTAAAGTTTCT |
| | | | | | | TCGCTCATGCAGACGAGAGCCATGGGCACGGGGCCGGCACCAG |
| | | | | | | GAAGAAGCGCGTTCATTCATTGGGTGTG |
| Hsl009b01 | Chr5 | 3000224 | CTTCGGTCTGTGTTGAAGG | CTGTTCTTCTCTGGGCTGG | GACATGAGATCCACAACCC | CTTCGGTCTGTGTTGAAGGGACAGCCCCCACCCGCTGTTCTTCT |
| | | | | | | CTGGGCTGGCGCTGAGCTTTGCCTGTGCATGGAATCACCCAGAG |
| | | | | | | CTGTCGGATGGGTTGTGGATCTCATGTC |
| Hsl009b02 | Chr5 | 3000686 | CTGTGGCTTGATTTCTTCCC | CCATGAATGCGGAGGAAGC | CTGTGTCCTTTGCTAGACG | CTGTGGCTTGATTTCTTCCCCCTCCCATGAATGCGGAGGAAGCC |
| | | | | | | GACTTTGAGAGATGAATGAACCATGTCAGTCCTGTCTTGAGAAGC |
| | | | | | | CCCTCGTCTAGCAAAGGACACAG |
| Hsl009b03 | Chr5 | 3002024 | GTACCAGTCAGGTTATGCC | CAGCTGAGTAACAAACATCC | GCCCTCTAGAAGAATCTTGC | GTACCAGTCAGGTTATGCCGTATTCAGCTGAGTAACAAACATCCT |
| | | | | | | CACAACTTCCAAGCCAGGCAGGAGCCCAGGGAGAGTTGGAACAT |
| | | | | | | GCAAGATTCTTCTAGAGGGC |
| Hsl009b04 | Chr5 | 3002784 | GATGCCAAAACTAAACTCTCC | CTCAGAGGTCCAAGAAAGC | GTCCAGAAACACCCACCC | GATGCCAAAACTAAACTCTCCTCTCAGAGGTCCAAGAAAGCACAT |
| | | | | | | TAGTTTTAAGATAAATCAGAGTTCCATTTCTGTCCTTGGACGTGTT |
| | | | | | | GGCAAGGGGGTGGGTGTTTCTGGAC |
| Hsl009b05 | Chr5 | 3003357 | CAAGCAGGAGAGGCATGC | GCTCTTGGAAGAACTTTAGG | CCTCTACAGATACATCATGC | CAAGGAGGAGAGGCATGCATTTGCTCTTGGAAGAACTTTAGGAA |
| | | | | | | CAGTCTTATGATGGGGGCTGCTTCCCACCCACAGCTTTTTGCATG |
| | | | | | | ATGTATCTGTAGAGG |
| Hsl009b06 | Chr5 | 3004394 | GGCCACAGCAATGTTGGG | GTTTCACTCTGGCTAACAGG | CCTGAATTGAATAGGCACCC | GGCCACAGCAATGTTGGGGAGTTTCACTCTGGCTAACAGGTTGG |
| | | | | | | TTTCTAACTCAAGTTTCCATTTAACCTCATAACTGAAAGGGGTGC |
| | | | | | | CTATTCAATTCAGG |
| Hsl009b07 | Chr5 | 174000178 | CACCTCAGAGCCAATAGCC | GAACAGCTGTTTGGACATGG | CAGAAGTCACCAGAGATCC | CACCTCAGAGCCAATAGCCCAGAACAGCTGTTTGGACATGGATT |
| | | | | | | GTTCTCTCTTTTGCTTCTGATGTGGAACTTTCTTTCCAGCAGGGAT |
| | | | | | | CTCTGGTGACTTCTG |
| Hsl009b08 | Chr5 | 174000879 | CAGCCTGAAACAACAACGG | GTCAAGGCAAGGGTAATCC | GACCAAGAAAGGCAGTAGC | CAGCCTGAAACAACAACGGATGGTCAACGCAAGGGTAATCCACC |
| | | | | | | AGAGGAACACTGAGCGGAGCTGTACCGCCCCAGCCACATCAGC |
| | | | | | | TACTGCCTTTCTTGGTC |
| Hsl009b09 | Chr5 | 174001818 | GAATGCAGCTTGATGATCCC | GGAGAGGAAGTGTCACAGG | GAGAGCCAAACACCTCCG | GAATGCAGCTTGATGATCCCAAATAACCAGGAGAGGAAGTGTCA |
| | | | | | | CAGGGTGAGGACAATGCAGAAACTACCCACTTCTTCCTGTGCCC |
| | | | | | | TTGATCCTCGGAGGTGTTTGGCTCTC |
| Hsl009b10 | Chr5 | 174002223 | GACAATGGAGGAAGTAGGC | GGACTGCCAGAGGCTTGG | CAATGGCATAGGCTTTTGG | GACAATGGAGGAAGTAGGCTGGACTGCCAGAGGCTTGGATTTTA |
| | | | | | | CTGACATCTGATCTTAGGGCATCACACATGGGTTGGCCATTTGAA |
| | | | | | | AGAATTCCAAAAGCCTATGCCATTG |
| Hsl009b11 | Chr5 | 174003634 | GGGTTGCTGGGAAACAGC | GCTTGACAACAGCAACAACC | GTGGACTCTTTTCTCATAACC | GGGTTGCTGGGAAACAGCATTTAAGACCTTGTTAACAATATGCTT |
| | | | | | | GACAACAGCAACAACCAGGCATAACATAACTAATAGTAGCATGCT |
| | | | | | | CTAATCAGCCCCCTATAGAGACAAGTCCAGGTTATGAGAAAAGA |
| | | | | | | GTCCAC |
| Hsl009b12 | Chr5 | 174004074 | CCAAGGAAGAAACCCATGC | GCGATGAAACCAGTATCCC | GTTTCAGTGTTTACATACTGG | CCAAGGAAGAAACCCATGCATAAGGCGATGAAACCAGTATCCCT |
| | | | | | | GTGACCTCTAGGCCGTTCGCTCTTAGACAGGCAGGTCCTTTGGG |
| | | | | | | TGATGGCCAGTATGTAAACACTGAAAC |
| Hsl009c01 | Chr6 | 5001280 | GCAAGTTTACTATCATCAAGC | CTCAGAACGGAACGTGACC | GAACTCTGTCTCTGAGAGG | GCAAGTTTACTATCATCAAGCAAAAAACTGACTCAGAACGGAACG |
| | | | | | | TGACCTTTGGGGATGCGCGGAGAGGGGTCCAAAGTAGAATTCTA |
| | | | | | | GATAAACATACACCTCTCAGAGACAGAGTTC |
| Hsl009c02 | Chr6 | 5001820 | GAAACAAACCAGTCCAACCC | GCAGATATGGGTGGAAATGG | GAGCAAAGTGCTTGTTGGG | GAAACAAACCAGTCCAACCCAGCAGATATGGGTGGAAATGGGGT |
| | | | | | | GAGTAGAGGAGGGGTTATGGCTACAAAATCTAAGCAGAAGACAC |
| | | | | | | TGGACCCCAACAAGCACTTTGCTC |
| Hsl009c03 | Chr6 | 5002147 | GAAACCACCACCTAAAGAGG | GAAAGAACAGGATGAGAATGC | CTGTTGTTTTGTTTCAAACAGG | GAAACCACCACCTAAAGAGGGTACAAGAAAGAACAGGATGAGAA |
| | | | | | | TGCGGGGGAGAACGCGTGTGCACCTGACCACACAGACTATACCA |
| | | | | | | CAGGCCCTGTTTGAAACAAAACAACAG |
| Hsl009c04 | Chr6 | 5003843 | GAGTGAGCAGCCAGAACG | CTCCAGACTGGGTACCGC | GAGTTGTAGTCTCTTAACTGC | GAGTGAGGAGCCAGAACGCCCCTGACAACAGCTCCCTCCAGACT |
| | | | | | | GGGTACCGCCCCCACGCCCGGCGCATCCTGGGAGTTGTAGTCC |
| | | | | | | TGTAGCCCTGCAAGCCACTGGCTTAGAGCAGTTAAGAGACTACA |
| | | | | | | ACTC |
| Hsl009c05 | Chr6 | 5004040 | CTAGCACTCTCCCCAAACC | CGAAAAGCCGAGGACAGC | CTTCGGCAACCACAAGTCG | CTAGCACTCTCCCCAAACCTCTCTCGCACGCGGGGACTGAGCAC |
| | | | | | | GGCCCGAAAAGCCGAGGACAGCCGGACTCACCCTGTAGTTATAG |
| | | | | | | TAGTGCGTCTGCACAAGATGCCGGTGGCGCGACTTGTGGTTGCC |
| | | | | | | GAAG |
| Hsl009c06 | Chr6 | 5004763 | CACACTGTTTGGTTCACAGG | CCATTGGGGACCTCTTGG | CAACCTTCCCTAATGTTTTGG | CACACTGTTTGGTTCACAGGACTCTGTTACCCATTGGGGACCTCT |
| | | | | | | TGGCCATTATTAACACAGGCCAACAGGACTAAAAGTTTGTATCAG |
| | | | | | | TCCTTCCCAAAACATTAGGGAAGGTTG |
| Hsl009c07 | Chr6 | 167002321 | GTGCTCACTGTCAACCCG | GCAGAGGCCATGCATAGG | GTCAGCCCTGAGAAAGCG | GTGCTCACTGTCAACCCGGCCAGCAGAGGCCATGCATAGGTGG |
| | | | | | | CCAGGTGCGACTACCTGTGTTCCAGCAAGTAGATGGAAAAGGAA |
| | | | | | | CACTGTCGCTTTCTCAGGGCTGAC |
| Hsl009c08 | Chr6 | 167002845 | CTAAGTATGCACTTTTGTGAG | CCATTACATATCCACACTGG | GCATAGAAGATACTCTGACC | CTAAGTATGCACTTTTGTGAGCACTTGTTCTAAATTATTGCCATTA |
| | | | C | | | CATATCCACACTGGAATTGAAAAATAACCCAGCTCAATTCATCGG |
| | | | | | | CCAAAGACACCCAGCCTCCATGGTCAGAGTATCTTCTATGC |
| Hsl009c09 | Chr6 | 167003342 | CTGTGGCATGAACAGAATGG | GAGACTTGGGATCTTACCG | GTGTATCTCACTTGCATGCC | CTGTGGCATGAACAGAATGGAGAGAGACTTGGGATCTTACCGGG |
| | | | | | | AGACAAGATCATACCCACCAACCCAACAAATGAGGCCACAGGCA |
| | | | | | | TGCAAGTGAGATACAC |
| Hsl009c10 | Chr6 | 167003857 | GATTCCCAGTGTGAACTCC | CAGACTCTGCTTTAGGAGG | GTTCTCACCCTAAGTCATGC | GATTCCCAGTGTGAACTCCGTGTCAGACTCTGCTTTAGGAGGAG |
| | | | | | | ACAGATCCTATTTCAGGGCTGGGCACACCTAAAGATGGAGCCTG |
| | | | | | | GCGAGGAGCATGACTTAGGGTGAGAAC |
| Hsl009c11 | Chr6 | 167004026 | CCTCATTGAGGACTTCAGG | GTTGCACTGTACTATACAGG | CTTTCAGTTATGCACGTGCG | CCTCATTGAGGACTTCAGGTCGTTGCACTGTACTATACAGGGGAT |
| | | | | | | TCGTGTGGAATGAGTTGATTGCTGCTGCTCTTGCCCCACAACACA |
| | | | | | | CACACGCACGTGCATAACTGAAAG |
| Hsl009c12 | Chr6 | 167004744 | CAACGCGACCAACAGTGC | CACTGGAGTGCCTTCTGG | CATCAAACCATGCCCATGC | CAACGCGACCAACAGTGCCACACTGGAGTGCCTTCTGGGATGAG |
| | | | | | | CAGAATGCCTTTAGACCAGTCACAGTGTGGCTGCTTCCGTCCAA |
| | | | | | | ATGGCGCTCGGCATGGGCATGGTTTGATG |
| Hsl009d01 | Chr7 | 24000376 | CTATGGATAACAAGCAGAGG | CAACCCACTTTTCATCAGC | GACTGAATGGTAACTGGACG | CTATGGATAACAAGGAGAGGTAACAACCCACTTTTCATCAGCATA |
| | | | | | | TTCTTTTTTCCAGAACACAATGCAATTACTGAGTGTGAGCTTCATC |
| | | | | | | GTCCAGTTACCATTCAGTC |
| Hsl009d02 | Chr7 | 24001065 | GTCAGGATCCTTGCAAAGC | CAAGTGCATGGTGAGATATGG | CATTACTCAAATGGGGTCTGG | GTCAGGATCCTTGCAAAGCAAGATAAGAGTAAATCAGATCAAGTG |
| | | | | | | CATGGTGAGATATGGCTGTATGAGAGTTTGCAGAGATATTTTTCT |
| | | | | | | TTTCCCTTCCAGACCCCATTTGAGTAATG |
| Hsl009d03 | Chr7 | 24002836 | GAACATCACTCTGGAAAGCC | CTGATGAGGCAATACATTGG | GAGGTTGACAGAGGGTAGG | GAACATCACTCTGGAAAGCCAGGGAGATTTTGTGCAAATCTGATG |
| | | | | | | AGGCAATACATTGGGAAATTAAACATGGTAATGACTCTCTGGTGA |
| | | | | | | ACTGATATACGACTCTCCTTTTACCTACCCTCTGTCAACCTC |
| Hsl009d04 | Chr7 | 24003315 | GATGACAACAGACTATTCGG | GAGTTCTCTGAAATGATTAGC | CTCCATTTGGGCTAGTGG | GATGACAACAGACTATTCGGAAGGTACTTTGTCTCAGAGTTCTCT |
| | | | | | | GAAATGATTAGCTATGTCTTACTTTTACCCGCTACTGAAGTGAAG |
| | | | | | | ATTGTAGGACACCTTCTCAGGCCCCCACTAGCCCAAATGGAG |
| Hsl009d05 | Chr7 | 24003547 | GTTCCCTCCTGTCTTTACG | CAGCTGTGTCTCAAGAGG | CATCTACACTAAGAAGAAGC | GTTCCCTCCTGTCTTTACGAACAGCTGTGTCTCAAGAGGTCACTG |
| | | | | | | AGGGAGCACTGGCTCTTCTCACAGCCAGCTCTCTCTTGAAGCTT |
| | | | | | | CTTCTTAGTGTAGATG |
| Hsl009d06 | Chr7 | 24004247 | GTGGAAAAGAAACCAGGCC | CAGTCTGAGGAGGAAAGAGG | CAACTTCAGCTAATCCATGC | GTGGAAAAGAAACCAGGCCCATTTTCAGCCAGTCTGAGGAGGAA |
| | | | | | | AGAGGTCCCTGAAGAGGCCTGGGGTTTGACTGCTGGGACCTAGT |
| | | | | | | GGGGCAAGTGGCATGGATTAGCTGAAGTTG |
| Hsl009d07 | Chr7 | 130001048 | GGCAACAGCTTTGAAAACC | GTCCATTCTTGTCCTGAAGG | GATCAATCTTATGCCAGAGG | GGCAACACCTTTGAAAACCAGTCCATTCTTGTCCTGAAGGTAAAA |
| | | | | | | GCCCACAATGTCAACCTGTAGACTCTACCTTGAGGGCCTCTGGC |
| | | | | | | ATAAGATTGATC |
| Hsl009d08 | Chr7 | 130001526 | CTTCACAGGAGCCACTGG | CAGAATTCAAGCAACTCAGG | CTAAGAATTGCTTTCTGATGG | CTTCACAGGAGCCACTGGAACAGAATTCAAGCAACTCAGGACCC |
| | | | | | | TGCAGTTCCTTGGGTCATGAAGGAAGTAGGAGATGTGAATGCAG |
| | | | | | | AGCCCATCAGAAAGCAATTCTTAG |
| Hsl009d09 | Chr7 | 130002216 | GTAGCCCAGAGACAGTAGC | GAGTTCAAACCTCGGTTTGG | GTGAATTCCAGTGTCAATCC | GTAGCCCAGAGACAGTAGCTGTCTGAGTTCAAACCTCGGTTTGG |
| | | | | | | GGCCCGATTCTTTTTCTCAGTTCAGCACTGGAGGTTCTCGGCAC |
| | | | | | | CCAGCCATCTGGATTGACACTGGAATTCAC |
| Hsl009d10 | Chr7 | 130002776 | CATGGACATCTTCATAGAGC | CAAACCCTGATGGGTTTGC | GTGACCTTTTCTCCATCCC | CATGGACATCTTCATAGAGCTCGTCACAAACCCTGATGGGTTTGC |
| | | | | | | TTTTACCCACAGCATGGTGAGGGAACCTGGGAAGGATGGAAGGA |
| | | | | | | GGGGGTCAGCTCTAGGGGGATGGAGAAAAGGTCAC |
| Hsl009d11 | Chr7 | 130003323 | CAACAGGAACTGGAAGTCG | GTTTTGGAGGTATGGCAACC | CTGACTGAGTGGGAGAACC | CAACAGGAACTGGAAGTCGGGTTTTGGAGGTATGGCAACCTGCT |
| | | | | | | GTCCTGGGGCAGGGTTGGAGAAGAGGTGTTGGCCCATGGCAGG |
| | | | | | | TTCTCCCACTCAGTCAG |
| Hsl009d12 | Chr7 | 130003733 | CTAGCCCTGCCCTGAAGG | GCACAACATGAAGAAATGCC | GGACACTTGAAACTATTGCC | CTAGCCCTGCCCTGAAGGGAGCACAACATGAAGAAATGCCTCTG |
| | | | | | | AACTCTTTCCCCGAGAGCTAGGACCTGAAATCTGCCCTCTGGGG |
| | | | | | | AGGCCAGGGCAATAGTTTCAAGTGTCC |
| Hsl009e01 | Chr8 | 10001122 | CAGAAGCAGCAAATGCAAGC | CTTTTGCAAGGAAATCAGGG | GTGATTGGAAACGAAAGTGG | CAGAAGCAGCAAATGCAAGCTGAAGTCTAACTTTTGCAAGGAAAT |
| | | | | | | CAGGGCTCCTTCTGGCTCCTCTGATATCTACCCTCATGACAGAAT |
| | | | | | | TCCAAGCAAGAGGGCCACTTTCGTTTCCAATCAC |
| Hsl009e02 | Chr8 | 10001529 | GATGGTGGCTTGCTTTTCC | GTCTGGTGGTAACAGTACC | CCTGACTTTCCTAAAGATGG | GATGGTGGCTTGCTTTTCCCATTTGTGAAGTCTGGTGGTAACAGT |
| | | | | | | ACCCAGACAGGGAAGTGAACAACCCTATAGTATAGTGACCGGAT |
| | | | | | | TTAGCAGGGCCGGATCGCCACCATCTTTAGGAAAGTCAGG |
| Hsl009e03 | Chr8 | 10002349 | CTTCTCTGTTAACTCTGTGC | GACAAGACACATGTAAACCC | CCAAAACGAGCCCAGCAGC | CTTCTCTGTTAACTCTGTGCCTTGATTGCTTAAGACAAGACACAT |
| | | | | | | GTAAACCCCATGATTATTGCCATTTTTTTGGACTTTGCAAAGACTC |
| | | | | | | TGCCTTCAAACATAAAGCTGCTGGGCTCGTTTTGG |
| Hsl009e04 | Chr8 | 10002667 | GTCAAACTCCAGGGACAGG | GCCTATGCAGTGCGAGGC | CTATTGTTTGTCTTAAGGAAGG | GTCAAACTCCAGGGACAGGCAGGGCCTATGCAGTGCGAGGCGA |
| | | | | | | GAACCGTCCGATCGGAGCACCTGTTCTATGTGGGGATCAGCTTT |
| | | | | | | TCCTTCCTTAAGACAAACAATAG |
| Hsl009e05 | Chr8 | 10003037 | GCATCCTCTGAAGAGGCG | GTTTGTGAGCACTCCATCC | GAAAGTGAACAGGTCACAGG | GCATCCTCTGAAGAGGCGTGTTTGTGAGCACTCCATCCACGGGG |
| | | | | | | CGGGTGGCCTTCTTGTACTTTTGATGTTTATACATTCTGATGATGT |
| | | | | | | GACCCTGTGACCTGTTCACTTTC |
| Hsl009e06 | Chr8 | 10003657 | GACCACATAACCCTAGAGC | GCAAAGAATGGTGCGATCG | CAGTTTACTCTAACATCACC | GACCACATAACCCTAGAGCAGCAAAGAATGGTGCGATCGTAAAG |
| | | | | | | GAAGAACCCATATTTGCTTTGGGNNNNNNNNCCCCTAGCTATTTG |
| | | | | | | GGTGATGTTAGAGTAAACTG |
| Hsl009e07 | Chr8 | 95001022 | GAATGTCAAGTGGATGTCC | CCTTCATCTGACATAGTTAGC | GCCAGAAACATCCATGGC | GAATGTCAAGTGGATGTCCAGACCTTCATCTGACATAGTTAGCTT |
| | | | | | | AGCAAAAACAAAAGTAAGATCTTTGTTCAGAGGGAGGAAATTCCA |
| | | | | | | TGCCATGGATGTTTCTGGC |
| Hsl009e08 | Chr8 | 95001714 | GGTTAGCAAAGCCTTCTCC | CCTTTCCTATTCTCAATGGC | CAGACTAAGTTCCTTGTTGC | GGTTAGCAAAGCCTTCTCCTGAATCCTTTCCTATTCTCAATGGCA |
| | | | | | | TGATATGTCAAGAACGTCTTTTGAGCCTGTTTGATCCAGTGATGT |
| | | | | | | TCAAATGTGCAACAAGGAACTTAGTCTG |
| Hsl009e09 | Chr8 | 95002098 | GACCTGTGTTTAGATGTGC | CTTCTGAAGGAAGTCATCCG | GACTTATGGTGGTCCTTAAGG | GACCTGTGTTTAGATGTGCTGTCACTTCTGAAGGAAGTCATCCGA |
| | | | | | | GCTTAAACTTATGGGATCTCACAAGGGGCCTGCAGTATCTCCTTA |
| | | | | | | AGGACCACCATAAGTC |
| Hsl009e10 | Chr8 | 95002612 | CGCTTACTGGAGACTGTGC | GCCAAGAGGTAATCTTCGG | GACTCTTAGGCAACTTGGC | CGCTTACTGGAGACTGTGCTCAAGAAAAAGCCAAGAGGTAATCTT |
| | | | | | | CGGCAGCTGCTGTGATATCTGCATATTTTAATTTTTTCCATCTATT |
| | | | | | | TAAAGCCTGCCAAGTTGCCTAAGAGTC |
| Hsl009e11 | Chr8 | 95003090 | GTAGCTGTTGTGGAGTAGC | GGTGAGGTGTATAGAGATCC | GCAATGTCCTGCCTTTTGC | GTAGCTGTTGTGGAGTAGCAGTGGGTGAGGTGTATAGAGATCCA |
| | | | | | | TTCATCCATGCAGCAAAACACTTGACTGGCTTGAGATGTGACATG |
| | | | | | | CGGAGCAAAAGGCAGGACATTGC |
| Hsl009e12 | Chr8 | 95003647 | GAATCTGAGGCTCAGGGC | GCAGAAGAGGGCTCTTGG | CTGAAATCAAAGGGGTTAGC | GAATCTGAGGCTCAGGGCAGCAGAAGAGGGCTCTTGGAGAAGA |
| | | | | | | GATGACAGTTGGCTGAAGTCGTCAACAGAGGGAGCTGGGAGGC |
| | | | | | | TGCTAACCCCTTTGATTTCAG |
| Hsl009f01 | Chr9 | 38000659 | GGCCTCCAAAGTCTTTGGG | CTGCTCCTCAATTCAGTCC | CCTATGTGGCAAGTAAAGCC | GGCCTCCAAAGTCTTTGGGGGCTGCTCCTCAATTCAGTCCTATAA |
| | | | | | | AGTGCATGGCATTTGGCCCTCGGAAGCCCCTCAAGGCTGAGAG |
| | | | | | | GCTTTACTTGCCACATAGG |
| Hsl009f02 | Chr9 | 38001855 | CCCAAAGGAGATGAACAGG | GAAGAGAAAAGGCCATCTGC | CTGTGAGGTGGGATCAGG | CCCAAAGGAGATGAACAGGAGAGAAGAGAAAAGGCCATCTGCAT |
| | | | | | | CCTCCCCATGAGCTCCAGAGAGCACGAGTGGTGGTGAGTGACTT |
| | | | | | | TCATCCACCCTGATCCCACCTCACAG |
| Hsl009f03 | Chr9 | 38002374 | GTGGAAAAGCCATCACTCC | GACCTAGAGGACAGGAACC | CGGTAGTGCTCTTTCAAAGC | GTGGAAAAGCCATCACTCCCTGCAGAGGACCTAGAGGACAGGAA |
| | | | | | | CCCTGACCGACCTGGACAGGGTTCCTACAGGGGAAGGCAGGGC |
| | | | | | | AGGAGGCCTTGCTTTGAAAGAGCACTACCG |
| Hsl009f04 | Chr9 | 38003209 | CAAAGGCATAGGGACCTGC | GCTTTTCACAATTCTGAGTCC | CAAGGGTGGAGTTGGAAGG | CAAAGGCATAGGGACCTGCCCCAGGTGGGTGCTTTTCACAATTC |
| | | | | | | TGAGTCCCCTTCAGCTAAACACAGGACCTCCTTGGGTTCTGTCCT |
| | | | | | | AGGCTGAGGCCTCTACTTCCTTCCAACTCCACCCTTG |
| Hsl009f05 | Chr9 | 38003751 | GCTCAGCACTAACCCTTCC | CCAGTAAAGACTCACTGAGC | CTTCCTTGACCTCTTCTAGG | GCTCAGCACTAACCCTTCCCCCAGTAAAGACTCACTCAGCAGAA |
| | | | | | | ACAGTTCTCCGTAAGGTAAAGGACACAGCACAGAAATGGAAGCA |
| | | | | | | AATCCTAGAAGAGGTCAAGGAAG |
| Hsl009f06 | Chr9 | 92000190 | GAATGTCCACACCAGGGG | CTTCATTGTAATGAGAAGTCC | CCATCGTGCTGTTCAGTGG | GAATGTCCACACCAGGGGCCCAATCTTCATTCTAATGAGAAGTCC |
| | | | | | | ACATTTTAGAGATGTTGTAGGTGCCTGCCCAGTCTGGCTGAGGC |
| | | | | | | CACTGAACAGCACGATGG |
| Hsl009f07 | Chr9 | 92000834 | CAGAGTCTCAGCCACAGG | CAGCTTTACAGATGAGACG | GTGCAGACTGCATCTGTGG | CAGAGTCTCAGCCACAGGTGGAGACAGCTTTACAGATGAGACGA |
| | | | | | | ACCACTTCTCTTGTGGATTTTCCACGGTGACGAGTCAGCTGTATC |
| | | | | | | ACTATCATATCCCACAGATGCAGTCTGCAC |
| Hsl009f08 | Chr9 | 92001027 | CCAACAACTCAATGACATTCC | CAACTTCGAAGAGAAAGTCC | GTTTGACACAGAGCCATTCC | CCAACAACTCAATGACATTCCAGCAACTTCGAAGAGAAAGTCCCG |
| | | | | | | TCTCCCCAGGTCTGCCTTCCTGCCTTCCCCAATTCAGATCCCACA |
| | | | | | | GCTCACGGAATGGCTCTGTGTCAAAC |
| Hsl009f09 | Chr9 | 92001631 | CTGTTCCATGGTTGACCCC | GAATCCTCACCAACAGTCG | GCACTTACCAGTGACACC | CTGTTCCATGGTTGACCCCAAGAATCCTCACCAACAGTCGACATT |
| | | | | | | ACACTTGAGGCTAAGTGCCACATGAGGGGGCTCCCATGCTCCAC |
| | | | | | | CAGCCCTCGGGGTGTCACTGGTAAGTGC |
| Hsl009F10 | Chr9 | 92002145 | CTTCTCAGAAATCTTCTTACG | CAAACCTCCCAGGTCACC | CCTCTGGTAGGAAAACTGG | CTTCTCAGAAATCTTCTTACGCTCCCACAAACCTCCCAGGTCACC |
| | | | | | | TCGAGGGAGGCAATGGAACTACTCACGAAAGAATAATTGGATTTC |
| | | | | | | CCAGTTTTCCTACCAGAGG |
| Hsl009f11 | Chr9 | 92002545 | CTGTTAACGTGCTCGTGTCC | CACGCAACGGGTGCTTCC | GCTACCCTCATTTCAAGGC | CTGTTAACGTGCTCGTGTCCCACCACGCAACGGGTGCTTCCACA |
| | | | | | | CAAGCAGCCAACGCAGGGGGGGCTGCAAAACCTGGAAACCACA |
| | | | | | | CAATGCATGCCTTGAAATGAGGGTAGC |
| Hsl009f12 | Chr9 | 92003110 | GCACATGCCTGTCACACC | CATGTGAGGGAAGGAATCG | CTGTCCACTAGTCAACAGG | GCACATGCCTGTCACACCCATTTCCCCATGTGAGGGAAGGAATC |
| | | | | | | GGCCTGGAAATTCCCAATTTCTACATAAAGTTCACTATATTTAGGA |
| | | | | | | GGAAAAAATGTGACTCCTGTTGACTAGTGGACAG |
| Hsl009g01 | ChrX | 40000047 | GGTAACTCTTGGAGCATGG | CACACTTATGACAAGTGAGC | GGCAATTGTGGACACTCG | GGTAACTCTTGGAGCATGGATGCCACACTTATGACAAGTGAGCA |
| | | | | | | GTGATTCTCAGCACAGAATGTGATATTTTTCTGTTGCACAAAGTTA |
| | | | | | | AACAGTGACCGAGTGTCCACAATTGCC |
| Hsl009g02 | ChrX | 40000788 | GTGCAGTGCTAAACCTTGG | CTCAGGTTTGTTTTGTTAAGG | CACAGCTTATCCCCAAAAGC | GTGCAGTGCTAAACCTTGGAGATTCTCAGGTTTGTTTTGTTAAGG |
| | | | | | | GAGGCAGTATTCCCTTACCAGCTCCCCCAGAGAGCCTACATTTG |
| | | | | | | TCCAGGAGCTTTTGGGGATAAGCTGTG |
| Hsl009g03 | ChrX | 40001110 | GGTAGGGTTTGGCTCAGG | CCATAGAGGGGTCCATTGC | CCAACCACTCTGGGTTCC | GGTAGGGTTTGGCTCAGGGAGGCCATAGAGGGGTCCATTGCTA |
| | | | | | | CAGGTTGCCCTCTGGCCTCGATGCCCACCTGTAAACTGCTATCTT |
| | | | | | | CAAGAGTGGAACCCAGAGTGGTTGG |
| Hsl009g04 | ChrX | 40001664 | GGTTGGGTCACTTCGATCC | GTGCTAGTAGGGTCTTTAGC | CAAGAGTCCAAGGACTAGG | GGTTGGGTCACTTCGATCCTGCCTGGGCCCAGGTGCTAGTAGG |
| | | | | | | GTCTTTAGCCTTCAGCTGAAGGTTCTCCCCTGCTCCTCCACCATC |
| | | | | | | TGTTTGGCTTTACAACACACACCTAGTCCTTGGACTCTTG |
| Hsl009g05 | ChrX | 40002012 | CCATTTCTCCTTGATTTCAGC | CCAAGTGAACATGCACTCC | GAAGAGAAAGTGAATCTTCCG | CCATTTCTCCTTGATTTCAGCACCCAAGTGAACATGCACTCCAAG |
| | | | | | | GCTCTGCTGAGGGTAAACAGAAAGCACCATCGCAGGGGTCCTTC |
| | | | | | | CTCCTCTCTCTCGGAAGATTCACTTTCTCTTC |
| Hsl009g06 | ChrX | 40003341 | GAAGGTGGTACAAGGAACC | CAGGAGACTGCAGTATCAGG | GAAGACTCTGGTGTTGTGC | GAAGGTGGTACAAGGAACCTGCAGGAGACTGCAGTATCAGGTG |
| | | | | | | GCAGTATCAGGAGGCTGATAAATCCAGGCTAATGGAAATTACTAT |
| | | | | | | TGCACAACACCAGAGTCTTC |
| Hsl009g07 | ChrX | 110001161 | CCCATGCTCTGGGTCTGG | CATGCCTCAACCTTCTTCC | CAGAAGTCTCCAAAAGTGG | CCCATGCTCTGGGTCTGGGTCATGCCTCAACCTTCTTCCCAGGG |
| | | | | | | AAGAACAATCTTTACACAGAAGTTTAGATAAGTTCCTATGACATTA |
| | | | | | | GACCACTTTTGGAGACTTCTG |
| Hsl009g08 | ChrX | 110001737 | GAGTTTGGGTGTTTCTTCTCC | GGAACATTTCAGTTGACTGG | GAAACCAAATGTATCCAGGC | GAGTTTGGGTGTTTCTTCTCCATTNNNNNNNNNTTCTCCACTCTT |
| | | | | | | GGAACATTTCAGTTGACTGGGTTTTCATTGAACCCCATTCGCAGC |
| | | | | | | CTTATTCCTAACATTTTTGCCTGGATACATTTGGTTTC |
| Hsl009g09 | ChrX | 110002145 | CCCAAGAGTGTCAAGTAGC | CTAGGATTGCCACTGGGC | CTTTGTTCATGTCTGACTGG | CCCAAGAGTGTCAAGTAGCTTTTTCTAGGATTGCCACTGGGCCC |
| | | | | | | ACAGGCATTCTCTGAATCACTCCACACGCTTTTGGGGTGGGAAT |
| | | | | | | CGGGCCCCAGTCAGACATGAACAAAG |
| Hsl009g10 | ChrX | 110003503 | GGACGAGCTAGAGTTTGC | GCTGATTAGGTAGTATGCC | GGTTGTGAGCTGTCAGAGC | GGACGAGCTAGAGTTTGGAATTTAGCTGATTAGGTAGTATGCCTG |
| | | | | | | GGTGGGGCGACTGGGTCCCTGCCTGATTTACAATTACAAGACCC |
| | | | | | | CTCGCTCTGACAGCTCACAACC |
| Hsl009g11 | ChrX | 110004046 | GTGTTGCATTTGGCAACACC | GTATCACACTCCTCAGAGG | GATTCACTTTAGACCTCAGC | GTGTTGCATTTGGCAACACCACAGAAGCTCCTCAGGTATCACACT |
| | | | | | | CCTCAGAGGCAGGTGGTATAATCTTGAATTGAGATCACTGAAGCA |
| | | | | | | CATCAGAAACCACACCTCCCAGCTGAGGTCTAAAGTGAATC |
| Hsl009g12 | ChrX | 110004631 | CTCTAGCTGGGCATGAGG | GTGCAGTCCTTACAAAAGG | GGAGGCCTTGTACTAGGC | CTCTAGCTGGGCATGAGGGAAGAGGTGCAGTCCTTACAAAAGGT |
| | | | | | | CTCAGGTAAGAAGCTGGTCTTGAAAATTCTTTGTGTAAGTTCAGA |
| | | | | | | ATTCTCAATGCCTAGTACAAGGCCTCC |
| Hsl009h01 | Y | 13400975 | GGTAAGAAAATGGTCCATCC | CCTATTCCACAGAAAGGATG | CAACATTAGAGACTATTCCAC | GGTAAGAAAATGGTCCATCCCCCTATTCCACAGAAAGGATGCTCA |
| | | | | | | TAACTACATGATGGATGAAAAAGAAAATATTAACAAATTCTGTTTG |
| | | | | | | CAAATCTAATATACTTTGTGGAATAGTCTCTAATGTTG |
| Hsl009h02 | Y | 13401213 | GTCAGGGTTCTTTCAAGGC | CAGTGATGAACAACAGTCTC | GGTATATCCAGTAATGAAAGG | GTCAGGGTTCTTTCAAGGCTCCCAGTGATGAACAACAGTCTCCTA |
| | | | | | | CCTCATCCATCTATCAAAGAAAACTCACCCTCAAGGTTTACCTTTC |
| | | | | | | ATTACTGGATATACC |
| Hsl009h03 | Y | 13401686 | GTGCTTTGTTCTCTTTGACAC | CTATCATTCTGGGACTTCTG | CTCAAGAAAGATGCAAGACC | GTGCTTTGTTCTCTTTGACACAGCTATCATTCTGGGACTTCTGTAT |
| | | | | | | ACAGCCTTTCCTTTGGTGGTCTTCTGGTGCTCCTTGGTCTTGCAT |
| | | | | | | CTTTCTTGAG |
| Hsl009h04 | Y | 13400027 | CCGTAATCATTACAATGATGG | CCCAATCTAGAGGTGGAAAG | GAACTATTCTACACATTTCTTC | CCGTAATCATTACAATGATGGTCCCAATCTAGAGGTGGAAAGTTG |
| | | | | | | TTTGCCTGGGGTGGTGAGTAATTCTCTATTCAAAATATGAAGAAA |
| | | | | | | TGTGTAGAATAGTTC |
| Hsl009h05 | Y | 13400391 | CTCATATGTAAAGGAACAACA | CTACCTTTCTTAGCCTTTCC | GACTTAAACCTCCCTAATGC | CTCATATGTAAAGGAACAACAGCTTCTACCTTTCTTAGCCTTTCCC |
| | | | G | | | TCAGCCTCTTAAAAATTATGCCTACAATTATACCAGTCACTTCAGC |
| | | | | | | ATTAGGGAGGTTTAAGTC |
| Hsl009h06 | Y | 13594365 | GAAGGGATGAATTACAAAGTG | GTGAGAAATGTTTGAGTGATG | CTGAAGCATGATATACAACAC | GAAGGGATGAATTACAAAGTGGTGTGAGAAATGTTTGAGTGATG |
| | | | | | | GAAGCTTTTGTTGTCTTTGTCAAAATGATAAAATTGTACAATAAAA |
| | | | | | | ATGTGTTGTATATCATGCTTCAG |
| Hsl009h07 | Y | 13597957 | GCTAAGTCAAAGAACAAGGG | GCTATCAGGGTCAACCAAG | GGCTATTGTTACCTCAGTTG | GCTAAGTCAAAGAACAAGGGTGGCTATCAGGGTCAACCAAGCAG |
| | | | | | | CAAGGTGCCAAGGCAGTCCCCAGGGGTTGTTTGCAGAGGATACT |
| | | | | | | GGCACACTTACACACACAACTGAGGTAACAATAGCC |
| Hsl009h08 | Y | 13595748 | GGTAATGTAGATAAGGTATCC | CAGCACCCTGATCAATAAGG | CTCTGTACCACATGAGTATC | GGTAATGTAGATAAGGTATCCCTCAGCACCCTGATCAATAAGGAA |
| | | | | | | TCACTTTTCACATTATATTGTTTAACAAATTCTATGCTCCAACTGCT |
| | | | | | | CCAAATTATGGATACTCATGTGGTACAGAG |
| Hsl009h09 | Y | 13598283 | CAACAGCAGCATCTCATGC | CTGAAACTCTAATAGACAAGC | GTGTTTATCTTCTAAAAGTGAC | CAACAGCAGCATCTCATGCATCTGAAACTCTAATAGACAAGCCAC |
| | | | | | | AATTTCTGGGAGCTAACTATGGCTTCCAGGCCTGGGTCACTTTTA |
| | | | | | | GAAGATAAACAC |
| Hsl009h10 | Y | 13595894 | GTGAGAAATGCTGAGGTCAC | CAGTTGGGTCAATGGTCAG | GGTCATAATGCCCAAACTTG | GTGAGAAATGCTGAGGTCACTGCAGTTGGGTCAATGGTCAGGAG |
| | | | | | | ACAGTAAAGAATTTCATGGAAAGAAGAAGCCTGTCAGCAGACTTC |
| | | | | | | AAAACAAGTTTGGGCATTATGACC |
| Hsl009h11 | Y | 15681453 | GGTTTCATTTGACTGTAAAGC | GTATCTCCTTCTTTCTTGGC | CCATTCTTTCACTAACATGAG | GGTTTCATTTGACTGTAAAGCTGTATCTCCTTCTTTCTTGGCATGT |
| | | | | | | AAAGATGGCAGGTGGAGCATTCTTTGCCTGCTACCCTCTCCCAG |
| | | | | | | CCACTCTCATGTTAGTGAAAGAATGG |
| Hsl009h12 | Y | 15630997 | GAGAAATAGCCTTCAAGGAG | CAGTTCATGATAGCTTGCTG | GTTCTCATGAAATCCTTGGG | GAGAAATAGCCTTCAAGGAGACAGTTCATGATAGCTTGCTGTTTA |
| | | | | | | AAGTGTTCTTATTTAAATTCCCAAGGATTTCATGAGAAC |

1. A method for counting the absolute copy number of a nucleic acid sequence in a cell, comprising the following steps:
(i) dividing a lysate of the cell or a lysate of a sample of the cell into a plurality of aliquots;
(ii) providing conditions suitable for the amplification of the nucleic acid sequence in each aliquot;
(iii) counting the number of aliquots in which the nucleic acid was amplified in step (ii) thus the copy number of the nucleic acid sequence in the cell.

2. The method according to claim 1, wherein, in step (i) the lysate is divided into at least 8 aliquots per cell used to make the lysate.

3. The method according to claim 1, wherein the lysate of step (i) is from a sample of 10 or fewer cells.

4. The method according to claim 1, wherein the lysate of step (i) is from a single cell.

5. A method for counting the absolute copy number of a chromatid in a cell comprising:
(i) dividing a lysate of the cell or a lysate of a sample of the cell into a plurality of aliquots;
(ii) providing conditions suitable for the amplification of one or more nucleic acid marker(s) of the chromatid in each aliquot;
(iii) counting the number of aliquots in which the nucleic acid marker was amplified in step (ii) thus the copy number of the chromatid in the cell.

6. The method according to claim 5, wherein the copy number of a plurality of nucleic acid markers from the chromatid is determined in order to analyse multiple loci on the chromatid.

7. The method according to claim 6, wherein the plurality of nucleic acid markers comprises one or more pairs or multiples of markers which occur in close proximity on the chromatid.

8. The method according to claim 6, wherein the highest number indicated by the plurality of nucleic acid markers gives the absolute copy number of the chromatid in the cell.

9. The method according to claim 5, wherein the chromatid is from chromosome 21, 18 or 13.

10. The method according to claim 9, further comprising repeating steps (i)-(iii) for chromatids for each of chromosome 21, 18 and 13.

11. (canceled)

12. A method for investigating the ploidy status of a cell, comprising counting the absolute copy number of chromatids for each chromosome in the cell by the method according to claim 5.

13. The method according to claim 1, wherein the cell is a polar body.

14. The method according to claim 1, wherein the cell is derived from a cleavage stage embryo.

15. The method according to claim 1, where wherein the cell is a trophectoderm cell of a blastocyst.

16. The method according to claim 1, wherein the cell is a fetal cell from an amniotic fluid or chorionic villus sample.

17. The method according to claim 16, wherein the cell is in telophase.

18. The method of claim 5, wherein the cell is an oocyte.

19-22. (canceled)

23. The method according to claim 13, wherein the ploidy status of both polar body I and polar body II are investigated.

24. (canceled)

25. The method according to claim 5, wherein the amplification of the one or more nucleic acid marker(s) of the chromatid is performed with a plurality of primers capable of amplifying the nucleic acid markers from the chromatid.

26-29. (canceled)

30. A primer set comprising one or more primer(s) from those listed in Table 2.


SEQUENCE LISTING (1152 sequences)

SEQ ID NO: 1 - DNA, 20 bp, Artificial Sequence
  External forward primer
catgaagttatggggttagg

SEQ ID NO: 2 - DNA, 20 bp, Artificial Sequence
  External forward primer
gaaccatctctttctttccc

SEQ ID NO: 3 - DNA, 18 bp, Artificial Sequence
  External forward primer
gccaacagagacctgacc

SEQ ID NO: 4 - DNA, 19 bp, Artificial Sequence
  External forward primer
cctgtttacagccctttcc

SEQ ID NO: 5 - DNA, 19 bp, Artificial Sequence
  External forward primer
gagcttctgttgagtgacc

SEQ ID NO: 6 - DNA, 18 bp, Artificial Sequence
  External forward primer
cttggaggcagcatgtgg

SEQ ID NO: 7 - DNA, 19 bp, Artificial Sequence
  External forward primer
ctaccctctagtgatgagg

SEQ ID NO: 8 - DNA, 18 bp, Artificial Sequence
  External forward primer
caccctccttggtaagcc

SEQ ID NO: 9 - DNA, 18 bp, Artificial Sequence
  External forward primer
gttgggctggtgcttggc

SEQ ID NO: 10 - DNA, 20 bp, Artificial Sequence
  External forward primer
cagatgaggaaaccaaaggg

SEQ ID NO: 11 - DNA, 20 bp, Artificial Sequence
  External forward primer
gacaccatcacgttttcagc

SEQ ID NO: 12 - DNA, 18 bp, Artificial Sequence
  External forward primer
ggtgacatggtactaggg

SEQ ID NO: 13 - DNA, 20 bp, Artificial Sequence
  External forward primer
catgtgagtggctatacaag

SEQ ID NO: 14 - DNA, 20 bp, Artificial Sequence
  External forward primer
gtcactctgaatatctgagg

SEQ ID NO: 15 - DNA, 21 bp, Artificial Sequence
  External forward primer
ggtagaatagaaagaaacacc

SEQ ID NO: 16 - DNA, 21 bp, Artificial Sequence
  External forward primer
ggtatgtgaatctatttgcac

SEQ ID NO: 17 - DNA, 20 bp, Artificial Sequence
  External forward primer
gaacatacagaaggctatgc

SEQ ID NO: 18 - DNA, 20 bp, Artificial Sequence
  External forward primer
cactcaaccattcagtcttc

SEQ ID NO: 19 - DNA, 21 bp, Artificial Sequence
  External forward primer
cttgtaagttccaacatcttc

SEQ ID NO: 20 - DNA, 20 bp, Artificial Sequence
  External forward primer
cctcaacagcatgaattagc

SEQ ID NO: 21 - DNA, 20 bp, Artificial Sequence
  External forward primer
gtagaccaaaggaagaatgg

SEQ ID NO: 22 - DNA, 19 bp, Artificial Sequence
  External forward primer
cagctgatcaagtgaagcg

SEQ ID NO: 23 - DNA, 20 bp, Artificial Sequence
  External forward primer
gatcttcatggacacaagtc

SEQ ID NO: 24 - DNA, 20 bp, Artificial Sequence
  External forward primer
gatgagtgcagatttgaagg

SEQ ID NO: 25 - DNA, 19 bp, Artificial Sequence
  External forward primer
cagctttgctttgcttggg

SEQ ID NO: 26 - DNA, 19 bp, Artificial Sequence
  External forward primer
gtgctgcattagagtttgg

SEQ ID NO: 27 - DNA, 21 bp, Artificial Sequence
  External forward primer
gtatgatagagttttccttcc

SEQ ID NO: 28 - DNA, 21 bp, Artificial Sequence
  External forward primer
cagatgtgttttgatttcagc

SEQ ID NO: 29 - DNA, 19 bp, Artificial Sequence
  External forward primer
gcaagacttcctcgtttgg

SEQ ID NO: 30 - DNA, 19 bp, Artificial Sequence
  External forward primer
ctgcagtttgccaaagtcg

SEQ ID NO: 31 - DNA, 18 bp, Artificial Sequence
  External forward primer
cttgcaggccatggaagg

SEQ ID NO: 32 - DNA, 19 bp, Artificial Sequence
  External forward primer
ctggagctcctgaattggg

SEQ ID NO: 33 - DNA, 19 bp, Artificial Sequence
  External forward primer
gatgcacctgtgctattgc

SEQ ID NO: 34 - DNA, 19 bp, Artificial Sequence
  External forward primer
gcaaacacctacacgttgg

SEQ ID NO: 35 - DNA, 20 bp, Artificial Sequence
  External forward primer
ggtattgttgtcatccaagc

SEQ ID NO: 36 - DNA, 19 bp, Artificial Sequence
  External forward primer
ctccagatgcctcaacagg

SEQ ID NO: 37 - DNA, 21 bp, Artificial Sequence
  External forward primer
cacaaaactaaagttgactcc

SEQ ID NO: 38 - DNA, 20 bp, Artificial Sequence
  External forward primer
gttaccaccttccctcttgc

SEQ ID NO: 39 - DNA, 20 bp, Artificial Sequence
  External forward primer
gagttttcaacctggctagc

SEQ ID NO: 40 - DNA, 20 bp, Artificial Sequence
  External forward primer
ctgagtatgcaaacagcacc

SEQ ID NO: 41 - DNA, 19 bp, Artificial Sequence
  External forward primer
cagttccacctttccaggc

SEQ ID NO: 42 - DNA, 20 bp, Artificial Sequence
  External forward primer
catccttctgtttcatagcc

SEQ ID NO: 43 - DNA, 21 bp, Artificial Sequence
  External forward primer
gtttccttcattccatgttcc

SEQ ID NO: 44 - DNA, 20 bp, Artificial Sequence
  External forward primer
caaagtgactgtgtccaagc

SEQ ID NO: 45 - DNA, 19 bp, Artificial Sequence
  External forward primer
cccagctatgagaagtacg

SEQ ID NO: 46 - DNA, 20 bp, Artificial Sequence
  External forward primer
gagtagtcaaggcctatagg

SEQ ID NO: 47 - DNA, 20 bp, Artificial Sequence
  External forward primer
gttcaaacagctaacaaccc

SEQ ID NO: 48 - DNA, 19 bp, Artificial Sequence
  External forward primer
cactcccttctggcagagg

SEQ ID NO: 49 - DNA, 20 bp, Artificial Sequence
  External forward primer
caatgtctcctaacagttgg

SEQ ID NO: 50 - DNA, 20 bp, Artificial Sequence
  External forward primer
ggatgacatcattccgaagg

SEQ ID NO: 51 - DNA, 18 bp, Artificial Sequence
  External forward primer
cactcccttggctatccg

SEQ ID NO: 52 - DNA, 20 bp, Artificial Sequence
  External forward primer
gctgtcaaacttcaacttgc

SEQ ID NO: 53 - DNA, 20 bp, Artificial Sequence
  External forward primer
gcaagggtcaaacttcaacc

SEQ ID NO: 54 - DNA, 19 bp, Artificial Sequence
  External forward primer
ctcctccagcagcaaaagg

SEQ ID NO: 55 - DNA, 21 bp, Artificial Sequence
  External forward primer
ccatactttagataggttacc

SEQ ID NO: 56 - DNA, 20 bp, Artificial Sequence
  External forward primer
ccagagactaagtcagaagc

SEQ ID NO: 57 - DNA, 19 bp, Artificial Sequence
  External forward primer
gtgcaaagcaagcatcagg

SEQ ID NO: 58 - DNA, 20 bp, Artificial Sequence
  External forward primer
gtatcaaaggcagtggaagc

SEQ ID NO: 59 - DNA, 20 bp, Artificial Sequence
  External forward primer
cattctgcaactgcttttcc

SEQ ID NO: 60 - DNA, 19 bp, Artificial Sequence
  External forward primer
gtgtttgtagggtcccacg

SEQ ID NO: 61 - DNA, 20 bp, Artificial Sequence
  External forward primer
ggaacatctctgcatacagg

SEQ ID NO: 62 - DNA, 19 bp, Artificial Sequence
  External forward primer
cattccctaaccccacagc

SEQ ID NO: 63 - DNA, 21 bp, Artificial Sequence
  External forward primer
gtagaagcttcttttcttagc

SEQ ID NO: 64 - DNA, 20 bp, Artificial Sequence
  External forward primer
ctagaagagaaactacaagc

SEQ ID NO: 65 - DNA, 21 bp, Artificial Sequence
  External forward primer
gggtacaatgaactgtaatgg

SEQ ID NO: 66 - DNA, 19 bp, Artificial Sequence
  External forward primer
caaggatgcaacactgagg

SEQ ID NO: 67 - DNA, 19 bp, Artificial Sequence
  External forward primer
ctaccctttctccaactgc

SEQ ID NO: 68 - DNA, 19 bp, Artificial Sequence
  External forward primer
gagctgctagagcttttgc

SEQ ID NO: 69 - DNA, 19 bp, Artificial Sequence
  External forward primer
gaacctgaacgtgttgagg

SEQ ID NO: 70 - DNA, 20 bp, Artificial Sequence
  External forward primer
cagatcatagattgtggagg

SEQ ID NO: 71 - DNA, 18 bp, Artificial Sequence
  External forward primer
ctggagtagagtctgggc

SEQ ID NO: 72 - DNA, 18 bp, Artificial Sequence
  External forward primer
gggtgggacctagaaagc

SEQ ID NO: 73 - DNA, 19 bp, Artificial Sequence
  External forward primer
ctaactgtcacctccttgg

SEQ ID NO: 74 - DNA, 22 bp, Artificial Sequence
  External forward primer
catagaaatcctaacatcttcc

SEQ ID NO: 75 - DNA, 19 bp, Artificial Sequence
  External forward primer
caaggccttgatgtagtgc

SEQ ID NO: 76 - DNA, 20 bp, Artificial Sequence
  External forward primer
gtaacccgtctaagatgtgg

SEQ ID NO: 77 - DNA, 19 bp, Artificial Sequence
  External forward primer
ccctgctttgagtaactcc

SEQ ID NO: 78 - DNA, 21 bp, Artificial Sequence
  External forward primer
ctatccttcagttttctaacc

SEQ ID NO: 79 - DNA, 20 bp, Artificial Sequence
  External forward primer
gataggacccagtgtattgc

SEQ ID NO: 80 - DNA, 19 bp, Artificial Sequence
  External forward primer
ctaagacgaagtcctcagc

SEQ ID NO: 81 - DNA, 19 bp, Artificial Sequence
  External forward primer
gtgcactgtcaatacaacg

SEQ ID NO: 82 - DNA, 19 bp, Artificial Sequence
  External forward primer
gagctctggattcattccg

SEQ ID NO: 83 - DNA, 19 bp, Artificial Sequence
  External forward primer
gcagtcatagttcttgagg

SEQ ID NO: 84 - DNA, 19 bp, Artificial Sequence
  External forward primer
gctgatggtaatcatctgg

SEQ ID NO: 85 - DNA, 21 bp, Artificial Sequence
  External forward primer
catctgtcagcaaactgttcc

SEQ ID NO: 86 - DNA, 21 bp, Artificial Sequence
  External forward primer
ctatgggtatgatatgttcgg

SEQ ID NO: 87 - DNA, 20 bp, Artificial Sequence
  External forward primer
gtagggaacatgcaaatccc

SEQ ID NO: 88 - DNA, 20 bp, Artificial Sequence
  External forward primer
caaccactatgtcacaaagc

SEQ ID NO: 89 - DNA, 20 bp, Artificial Sequence
  External forward primer
gtcttcatccatcagactgg

SEQ ID NO: 90 - DNA, 20 bp, Artificial Sequence
  External forward primer
cactcaatagactttcaggg

SEQ ID NO: 91 - DNA, 19 bp, Artificial Sequence
  External forward primer
gaggctataggttaagagg

SEQ ID NO: 92 - DNA, 20 bp, Artificial Sequence
  External forward primer
gtctccactggaagaagagc

SEQ ID NO: 93 - DNA, 20 bp, Artificial Sequence
  External forward primer
cctgttttcccaagtttacc

SEQ ID NO: 94 - DNA, 19 bp, Artificial Sequence
  External forward primer
ggtgttaggttcccacagg

SEQ ID NO: 95 - DNA, 20 bp, Artificial Sequence
  External forward primer
caagtgaatgagtgaatggg

SEQ ID NO: 96 - DNA, 18 bp, Artificial Sequence
  External forward primer
gagcagtcaggggactcc

SEQ ID NO: 97 - DNA, 19 bp, Artificial Sequence
  External forward primer
ccagaccaagtgacagtgg

SEQ ID NO: 98 - DNA, 19 bp, Artificial Sequence
  External forward primer
ctggccatgagtactttcc

SEQ ID NO: 99 - DNA, 20 bp, Artificial Sequence
  External forward primer
ctcagcttttggaatgaagc

SEQ ID NO: 100 - DNA, 20 bp, Artificial Sequence
  External forward primer
caccatgtactcttcacagg

SEQ ID NO: 101 - DNA, 20 bp, Artificial Sequence
  External forward primer
gtggagttgatcatttgagg

SEQ ID NO: 102 - DNA, 20 bp, Artificial Sequence
  External forward primer
cactagtatgtagagtgtgg

SEQ ID NO: 103 - DNA, 21 bp, Artificial Sequence
  External forward primer
gtgttttgaagctaagatgcg

SEQ ID NO: 104 - DNA, 20 bp, Artificial Sequence
  External forward primer
gactaatgtaaaccacctgg

SEQ ID NO: 105 - DNA, 19 bp, Artificial Sequence
  External forward primer
cgtactatgtctgttcacc

SEQ ID NO: 106 - DNA, 19 bp, Artificial Sequence
  External forward primer
ggatgggaatggagtgacg

SEQ ID NO: 107 - DNA, 20 bp, Artificial Sequence
  External forward primer
gagctttcatttcacatggg

SEQ ID NO: 108 - DNA, 19 bp, Artificial Sequence
  External forward primer
ccattcatcccgtatcagg

SEQ ID NO: 109 - DNA, 20 bp, Artificial Sequence
  External forward primer
ctgggaataggatccttagg

SEQ ID NO: 110 - DNA, 19 bp, Artificial Sequence
  External forward primer
caagtctctgctgagaagg

SEQ ID NO: 111 - DNA, 21 bp, Artificial Sequence
  External forward primer
gtgaagtgattccaagaatcc

SEQ ID NO: 112 - DNA, 20 bp, Artificial Sequence
  External forward primer
gaagcaaatgttcagaaggg

SEQ ID NO: 113 - DNA, 19 bp, Artificial Sequence
  External forward primer
cagaggtggagtaaagtgg

SEQ ID NO: 114 - DNA, 19 bp, Artificial Sequence
  External forward primer
ctgctctcctagtgttgcc

SEQ ID NO: 115 - DNA, 19 bp, Artificial Sequence
  External forward primer
gtcgatgagtgaggtttcc

SEQ ID NO: 116 - DNA, 20 bp, Artificial Sequence
  External forward primer
caaggaaagctctgaattgc

SEQ ID NO: 117 - DNA, 19 bp, Artificial Sequence
  External forward primer
gaaccctgaaggcatagcc

SEQ ID NO: 118 - DNA, 19 bp, Artificial Sequence
  External forward primer
ctctggccattgactttgg

SEQ ID NO: 119 - DNA, 19 bp, Artificial Sequence
  External forward primer
ccagttctcaccggaaagg

SEQ ID NO: 120 - DNA, 18 bp, Artificial Sequence
  External forward primer
gcatccagggctgaaacc

SEQ ID NO: 121 - DNA, 19 bp, Artificial Sequence
  External forward primer
gccaatgcatttccaagcc

SEQ ID NO: 122 - DNA, 19 bp, Artificial Sequence
  External forward primer
cttgcccatggaatgaagc

SEQ ID NO: 123 - DNA, 19 bp, Artificial Sequence
  External forward primer
gatccaggtgtatctctgc

SEQ ID NO: 124 - DNA, 18 bp, Artificial Sequence
  External forward primer
gacgacatcggaggatcc

SEQ ID NO: 125 - DNA, 19 bp, Artificial Sequence
  External forward primer
gcctaacatggcgtgtagg

SEQ ID NO: 126 - DNA, 19 bp, Artificial Sequence
  External forward primer
ccagacatgagcaaacagc

SEQ ID NO: 127 - DNA, 20 bp, Artificial Sequence
  External forward primer
ctgtctttccaccaaactgg

SEQ ID NO: 128 - DNA, 19 bp, Artificial Sequence
  External forward primer
gttctagggctgacagacc

SEQ ID NO: 129 - DNA, 18 bp, Artificial Sequence
  External forward primer
gatgcccagctgctgagg

SEQ ID NO: 130 - DNA, 20 bp, Artificial Sequence
  External forward primer
cagttcctcatgtacagtcc

SEQ ID NO: 131 - DNA, 18 bp, Artificial Sequence
  External forward primer
ccttcatgcctgcttggg

SEQ ID NO: 132 - DNA, 20 bp, Artificial Sequence
  External forward primer
caggcattgtatgaagttcc

SEQ ID NO: 133 - DNA, 19 bp, Artificial Sequence
  External forward primer
catgaccttcttagagacc

SEQ ID NO: 134 - DNA, 19 bp, Artificial Sequence
  External forward primer
ctgagtccgaattcaagcc

SEQ ID NO: 135 - DNA, 19 bp, Artificial Sequence
  External forward primer
ggctttgggacaagattcc

SEQ ID NO: 136 - DNA, 19 bp, Artificial Sequence
  External forward primer
cgttagcacaacccatggc

SEQ ID NO: 137 - DNA, 18 bp, Artificial Sequence
  External forward primer
cccatcctccttgcatgg

SEQ ID NO: 138 - DNA, 19 bp, Artificial Sequence
  External forward primer
ggctgtcttctttgtctcc

SEQ ID NO: 139 - DNA, 19 bp, Artificial Sequence
  External forward primer
gcagttagggaaggttccc

SEQ ID NO: 140 - DNA, 19 bp, Artificial Sequence
  External forward primer
caagccacaaactgtaggg

SEQ ID NO: 141 - DNA, 21 bp, Artificial Sequence
  External forward primer
gtgtgattactcactaatccc

SEQ ID NO: 142 - DNA, 20 bp, Artificial Sequence
  External forward primer
ctgtcattgtaacgtttccc

SEQ ID NO: 143 - DNA, 20 bp, Artificial Sequence
  External forward primer
cttgtgacttacccttacgc

SEQ ID NO: 144 - DNA, 20 bp, Artificial Sequence
  External forward primer
caggttagtagtaccatggc

SEQ ID NO: 145 - DNA, 19 bp, Artificial Sequence
  External forward primer
gttccgtccgattcttccc

SEQ ID NO: 146 - DNA, 19 bp, Artificial Sequence
  External forward primer
ctgattctatgggcagcgc

SEQ ID NO: 147 - DNA, 20 bp, Artificial Sequence
  External forward primer
ctccaatactgcacaatccg

SEQ ID NO: 148 - DNA, 18 bp, Artificial Sequence
  External forward primer
ctttcgtagacagcagcc

SEQ ID NO: 149 - DNA, 19 bp, Artificial Sequence
  External forward primer
cagctccacaactagtagg

SEQ ID NO: 150 - DNA, 19 bp, Artificial Sequence
  External forward primer
caaccacattgatgtgagc

SEQ ID NO: 151 - DNA, 18 bp, Artificial Sequence
  External forward primer
ctgcagcacctgtcatgg

SEQ ID NO: 152 - DNA, 19 bp, Artificial Sequence
  External forward primer
ctttgctcagaccaacacg

SEQ ID NO: 153 - DNA, 19 bp, Artificial Sequence
  External forward primer
cagctcaggatggaaaagg

SEQ ID NO: 154 - DNA, 18 bp, Artificial Sequence
  External forward primer
cagagcaagagggatggg

SEQ ID NO: 155 - DNA, 20 bp, Artificial Sequence
  External forward primer
gagggaccaaactatgaagg

SEQ ID NO: 156 - DNA, 20 bp, Artificial Sequence
  External forward primer
gaagtgtcaacagcatagcc

SEQ ID NO: 157 - DNA, 19 bp, Artificial Sequence
  External forward primer
gcatccacacgtgatgtgc

SEQ ID NO: 158 - DNA, 19 bp, Artificial Sequence
  External forward primer
ctaacctattgccagctgc

SEQ ID NO: 159 - DNA, 19 bp, Artificial Sequence
  External forward primer
ggactgcagctagtatggc

SEQ ID NO: 160 - DNA, 20 bp, Artificial Sequence
  External forward primer
ctttgttaagctcactttgc

SEQ ID NO: 161 - DNA, 18 bp, Artificial Sequence
  External forward primer
caggatgtgaccactggc

SEQ ID NO: 162 - DNA, 19 bp, Artificial Sequence
  External forward primer
ccacagacagttctagagg

SEQ ID NO: 163 - DNA, 20 bp, Artificial Sequence
  External forward primer
gaagtttctgggacacaaag

SEQ ID NO: 164 - DNA, 21 bp, Artificial Sequence
  External forward primer
gtcattgctggaaattgattc

SEQ ID NO: 165 - DNA, 20 bp, Artificial Sequence
  External forward primer
cttcttctcttcaagggtag

SEQ ID NO: 166 - DNA, 20 bp, Artificial Sequence
  External forward primer
cgtcaacacggattacattc

SEQ ID NO: 167 - DNA, 18 bp, Artificial Sequence
  External forward primer
caccagccagcattcagc

SEQ ID NO: 168 - DNA, 18 bp, Artificial Sequence
  External forward primer
ctccagcctgtctgtagg

SEQ ID NO: 169 - DNA, 19 bp, Artificial Sequence
  External forward primer
gtaagccctgtggttctgg

SEQ ID NO: 170 - DNA, 20 bp, Artificial Sequence
  External forward primer
gctcaatgacaatgctgtcc

SEQ ID NO: 171 - DNA, 18 bp, Artificial Sequence
  External forward primer
gctctgggtcatcttccc

SEQ ID NO: 172 - DNA, 18 bp, Artificial Sequence
  External forward primer
cacctctggagggagtgc

SEQ ID NO: 173 - DNA, 19 bp, Artificial Sequence
  External forward primer
gaccagacctctaaacacc

SEQ ID NO: 174 - DNA, 20 bp, Artificial Sequence
  External forward primer
gcatacgaattcccaaatcc

SEQ ID NO: 175 - DNA, 20 bp, Artificial Sequence
  External forward primer
gtttggagggatggaaatgg

SEQ ID NO: 176 - DNA, 20 bp, Artificial Sequence
  External forward primer
ctgtgaggatgatggacagg

SEQ ID NO: 177 - DNA, 19 bp, Artificial Sequence
  External forward primer
cagtcatcttccaagttgc

SEQ ID NO: 178 - DNA, 19 bp, Artificial Sequence
  External forward primer
gagagcagagggcttctgg

SEQ ID NO: 179 - DNA, 20 bp, Artificial Sequence
  External forward primer
cagtggattagcctaaacgc

SEQ ID NO: 180 - DNA, 20 bp, Artificial Sequence
  External forward primer
cttcatgctctcatcaaacc

SEQ ID NO: 181 - DNA, 20 bp, Artificial Sequence
  External forward primer
gttgacaagtagtgggttcc

SEQ ID NO: 182 - DNA, 19 bp, Artificial Sequence
  External forward primer
gtgaatgacatgggtgagg

SEQ ID NO: 183 - DNA, 18 bp, Artificial Sequence
  External forward primer
cacagacagctgctcagg

SEQ ID NO: 184 - DNA, 20 bp, Artificial Sequence
  External forward primer
gagcaaagctaatccattcc

SEQ ID NO: 185 - DNA, 19 bp, Artificial Sequence
  External forward primer
gcgcttgtctcttttctgg

SEQ ID NO: 186 - DNA, 19 bp, Artificial Sequence
  External forward primer
gttggtcctccatagaagc

SEQ ID NO: 187 - DNA, 19 bp, Artificial Sequence
  External forward primer
gtatccccttcacttctgg

SEQ ID NO: 188 - DNA, 20 bp, Artificial Sequence
  External forward primer
gaacatgggatgaactcagc

SEQ ID NO: 189 - DNA, 21 bp, Artificial Sequence
  External forward primer
gaggtgaagatcattctaacc

SEQ ID NO: 190 - DNA, 20 bp, Artificial Sequence
  External forward primer
ccaaaaccattcacttaggg

SEQ ID NO: 191 - DNA, 19 bp, Artificial Sequence
  External forward primer
gccatcttccaggttttcc

SEQ ID NO: 192 - DNA, 19 bp, Artificial Sequence
  External forward primer
gaagagcctgtttcagtgg

SEQ ID NO: 193 - DNA, 20 bp, Artificial Sequence
  External forward primer
gtagaagaaagatccacccc

SEQ ID NO: 194 - DNA, 19 bp, Artificial Sequence
  External forward primer
cagctgggaatgtgatacc

SEQ ID NO: 195 - DNA, 21 bp, Artificial Sequence
  External forward primer
caacagaaagaatagcttgcc

SEQ ID NO: 196 - DNA, 20 bp, Artificial Sequence
  External forward primer
gtgctactttcatggctagg

SEQ ID NO: 197 - DNA, 20 bp, Artificial Sequence
  External forward primer
gttacccacaaactcaacgg

SEQ ID NO: 198 - DNA, 20 bp, Artificial Sequence
  External forward primer
cttttcagcagacttttggc

SEQ ID NO: 199 - DNA, 19 bp, Artificial Sequence
  External forward primer
gtcatgacaacttctgtcc

SEQ ID NO: 200 - DNA, 18 bp, Artificial Sequence
  External forward primer
cccaacagggacatgtcc

SEQ ID NO: 201 - DNA, 21 bp, Artificial Sequence
  External forward primer
ccagtttcatagacatcttgc

SEQ ID NO: 202 - DNA, 20 bp, Artificial Sequence
  External forward primer
gggatgtgttgcacaaaagc

SEQ ID NO: 203 - DNA, 18 bp, Artificial Sequence
  External forward primer
ggttctcctgacctctcc

SEQ ID NO: 204 - DNA, 19 bp, Artificial Sequence
  External forward primer
cagctctaccaaccacagc

SEQ ID NO: 205 - DNA, 19 bp, Artificial Sequence
  External forward primer
cttcggtctgtgttgaagg

SEQ ID NO: 206 - DNA, 20 bp, Artificial Sequence
  External forward primer
ctgtggcttgatttcttccc

SEQ ID NO: 207 - DNA, 19 bp, Artificial Sequence
  External forward primer
gtaccagtcaggttatgcc

SEQ ID NO: 208 - DNA, 21 bp, Artificial Sequence
  External forward primer
gatgccaaaactaaactctcc

SEQ ID NO: 209 - DNA, 18 bp, Artificial Sequence
  External forward primer
caagcaggagaggcatgc

SEQ ID NO: 210 - DNA, 18 bp, Artificial Sequence
  External forward primer
ggccacagcaatgttggg

SEQ ID NO: 211 - DNA, 19 bp, Artificial Sequence
  External forward primer
cacctcagagccaatagcc

SEQ ID NO: 212 - DNA, 19 bp, Artificial Sequence
  External forward primer
cagcctgaaacaacaacgg

SEQ ID NO: 213 - DNA, 20 bp, Artificial Sequence
  External forward primer
gaatgcagcttgatgatccc

SEQ ID NO: 214 - DNA, 19 bp, Artificial Sequence
  External forward primer
gacaatggaggaagtaggc

SEQ ID NO: 215 - DNA, 18 bp, Artificial Sequence
  External forward primer
gggttgctgggaaacagc

SEQ ID NO: 216 - DNA, 19 bp, Artificial Sequence
  External forward primer
ccaaggaagaaacccatgc

SEQ ID NO: 217 - DNA, 21 bp, Artificial Sequence
  External forward primer
gcaagtttactatcatcaagc

SEQ ID NO: 218 - DNA, 20 bp, Artificial Sequence
  External forward primer
gaaacaaaccagtccaaccc

SEQ ID NO: 219 - DNA, 20 bp, Artificial Sequence
  External forward primer
gaaaccaccacctaaagagg

SEQ ID NO: 220 - DNA, 18 bp, Artificial Sequence
  External forward primer
gagtgagcagccagaacg

SEQ ID NO: 221 - DNA, 19 bp, Artificial Sequence
  External forward primer
ctagcactctccccaaacc

SEQ ID NO: 222 - DNA, 20 bp, Artificial Sequence
  External forward primer
cacactgtttggttcacagg

SEQ ID NO: 223 - DNA, 18 bp, Artificial Sequence
  External forward primer
gtgctcactgtcaacccg

SEQ ID NO: 224 - DNA, 22 bp, Artificial Sequence
  External forward primer
ctaagtatgcacttttgtgagc

SEQ ID NO: 225 - DNA, 20 bp, Artificial Sequence
  External forward primer
ctgtggcatgaacagaatgg

SEQ ID NO: 226 - DNA, 19 bp, Artificial Sequence
  External forward primer
gattcccagtgtgaactcc

SEQ ID NO: 227 - DNA, 19 bp, Artificial Sequence
  External forward primer
cctcattgaggacttcagg

SEQ ID NO: 228 - DNA, 18 bp, Artificial Sequence
  External forward primer
caacgcgaccaacagtgc

SEQ ID NO: 229 - DNA, 20 bp, Artificial Sequence
  External forward primer
ctatggataacaagcagagg

SEQ ID NO: 230 - DNA, 19 bp, Artificial Sequence
  External forward primer
gtcaggatccttgcaaagc

SEQ ID NO: 231 - DNA, 20 bp, Artificial Sequence
  External forward primer
gaacatcactctggaaagcc

SEQ ID NO: 232 - DNA, 20 bp, Artificial Sequence
  External forward primer
gatgacaacagactattcgg

SEQ ID NO: 233 - DNA, 19 bp, Artificial Sequence
  External forward primer
gttccctcctgtctttacg

SEQ ID NO: 234 - DNA, 19 bp, Artificial Sequence
  External forward primer
gtggaaaagaaaccaggcc

SEQ ID NO: 235 - DNA, 19 bp, Artificial Sequence
  External forward primer
ggcaacagctttgaaaacc

SEQ ID NO: 236 - DNA, 18 bp, Artificial Sequence
  External forward primer
cttcacaggagccactgg

SEQ ID NO: 237 - DNA, 19 bp, Artificial Sequence
  External forward primer
gtagcccagagacagtagc

SEQ ID NO: 238 - DNA, 20 bp, Artificial Sequence
  External forward primer
catggacatcttcatagagc

SEQ ID NO: 239 - DNA, 19 bp, Artificial Sequence
  External forward primer
caacaggaactggaagtcg

SEQ ID NO: 240 - DNA, 18 bp, Artificial Sequence
  External forward primer
ctagccctgccctgaagg

SEQ ID NO: 241 - DNA, 20 bp, Artificial Sequence
  External forward primer
cagaagcagcaaatgcaagc

SEQ ID NO: 242 - DNA, 19 bp, Artificial Sequence
  External forward primer
gatggtggcttgcttttcc

SEQ ID NO: 243 - DNA, 20 bp, Artificial Sequence
  External forward primer
cttctctgttaactctgtgc

SEQ ID NO: 244 - DNA, 19 bp, Artificial Sequence
  External forward primer
gtcaaactccagggacagg

SEQ ID NO: 245 - DNA, 18 bp, Artificial Sequence
  External forward primer
gcatcctctgaagaggcg

SEQ ID NO: 246 - DNA, 19 bp, Artificial Sequence
  External forward primer
gaccacataaccctagagc

SEQ ID NO: 247 - DNA, 19 bp, Artificial Sequence
  External forward primer
gaatgtcaagtggatgtcc

SEQ ID NO: 248 - DNA, 19 bp, Artificial Sequence
  External forward primer
ggttagcaaagccttctcc

SEQ ID NO: 249 - DNA, 19 bp, Artificial Sequence
  External forward primer
gacctgtgtttagatgtgc

SEQ ID NO: 250 - DNA, 19 bp, Artificial Sequence
  External forward primer
cgcttactggagactgtgc

SEQ ID NO: 251 - DNA, 19 bp, Artificial Sequence
  External forward primer
gtagctgttgtggagtagc

SEQ ID NO: 252 - DNA, 18 bp, Artificial Sequence
  External forward primer
gaatctgaggctcagggc

SEQ ID NO: 253 - DNA, 19 bp, Artificial Sequence
  External forward primer
ggcctccaaagtctttggg

SEQ ID NO: 254 - DNA, 19 bp, Artificial Sequence
  External forward primer
cccaaaggagatgaacagg

SEQ ID NO: 255 - DNA, 19 bp, Artificial Sequence
  External forward primer
gtggaaaagccatcactcc

SEQ ID NO: 256 - DNA, 19 bp, Artificial Sequence
  External forward primer
caaaggcatagggacctgc

SEQ ID NO: 257 - DNA, 19 bp, Artificial Sequence
  External forward primer
gctcagcactaacccttcc

SEQ ID NO: 258 - DNA, 18 bp, Artificial Sequence
  External forward primer
gaatgtccacaccagggg

SEQ ID NO: 259 - DNA, 18 bp, Artificial Sequence
  External forward primer
cagagtctcagccacagg

SEQ ID NO: 260 - DNA, 21 bp, Artificial Sequence
  External forward primer
ccaacaactcaatgacattcc

SEQ ID NO: 261 - DNA, 19 bp, Artificial Sequence
  External forward primer
ctgttccatggttgacccc

SEQ ID NO: 262 - DNA, 21 bp, Artificial Sequence
  External forward primer
cttctcagaaatcttcttacg

SEQ ID NO: 263 - DNA, 20 bp, Artificial Sequence
  External forward primer
ctgttaacgtgctcgtgtcc

SEQ ID NO: 264 - DNA, 18 bp, Artificial Sequence
  External forward primer
gcacatgcctgtcacacc

SEQ ID NO: 265 - DNA, 19 bp, Artificial Sequence
  External forward primer
ggtaactcttggagcatgg

SEQ ID NO: 266 - DNA, 19 bp, Artificial Sequence
  External forward primer
gtgcagtgctaaaccttgg

SEQ ID NO: 267 - DNA, 18 bp, Artificial Sequence
  External forward primer
ggtagggtttggctcagg

SEQ ID NO: 268 - DNA, 19 bp, Artificial Sequence
  External forward primer
ggttgggtcacttcgatcc

SEQ ID NO: 269 - DNA, 21 bp, Artificial Sequence
  External forward primer
ccatttctccttgatttcagc

SEQ ID NO: 270 - DNA, 19 bp, Artificial Sequence
  External forward primer
gaaggtggtacaaggaacc

SEQ ID NO: 271 - DNA, 18 bp, Artificial Sequence
  External forward primer
cccatgctctgggtctgg

SEQ ID NO: 272 - DNA, 21 bp, Artificial Sequence
  External forward primer
gagtttgggtgtttcttctcc

SEQ ID NO: 273 - DNA, 19 bp, Artificial Sequence
  External forward primer
cccaagagtgtcaagtagc

SEQ ID NO: 274 - DNA, 18 bp, Artificial Sequence
  External forward primer
ggacgagctagagtttgg

SEQ ID NO: 275 - DNA, 20 bp, Artificial Sequence
  External forward primer
gtgttgcatttggcaacacc

SEQ ID NO: 276 - DNA, 18 bp, Artificial Sequence
  External forward primer
ctctagctgggcatgagg

SEQ ID NO: 277 - DNA, 20 bp, Artificial Sequence
  External forward primer
ggtaagaaaatggtccatcc

SEQ ID NO: 278 - DNA, 19 bp, Artificial Sequence
  External forward primer
gtcagggttctttcaaggc

SEQ ID NO: 279 - DNA, 21 bp, Artificial Sequence
  External forward primer
gtgctttgttctctttgacac

SEQ ID NO: 280 - DNA, 21 bp, Artificial Sequence
  External forward primer
ccgtaatcattacaatgatgg

SEQ ID NO: 281 - DNA, 22 bp, Artificial Sequence
  External forward primer
ctcatatgtaaaggaacaacag

SEQ ID NO: 282 - DNA, 21 bp, Artificial Sequence
  External forward primer
gaagggatgaattacaaagtg

SEQ ID NO: 283 - DNA, 20 bp, Artificial Sequence
  External forward primer
gctaagtcaaagaacaaggg

SEQ ID NO: 284 - DNA, 21 bp, Artificial Sequence
  External forward primer
ggtaatgtagataaggtatcc

SEQ ID NO: 285 - DNA, 19 bp, Artificial Sequence
  External forward primer
caacagcagcatctcatgc

SEQ ID NO: 286 - DNA, 20 bp, Artificial Sequence
  External forward primer
gtgagaaatgctgaggtcac

SEQ ID NO: 287 - DNA, 21 bp, Artificial Sequence
  External forward primer
ggtttcatttgactgtaaagc

SEQ ID NO: 288 - DNA, 20 bp, Artificial Sequence
  External forward primer
gagaaatagccttcaaggag

SEQ ID NO: 289 - DNA, 19 bp, Artificial Sequence
  Internal forward primer
gctagtttcctcttgaagg

SEQ ID NO: 290 - DNA, 20 bp, Artificial Sequence
  Internal forward primer
ctctgcatacacttttctcg

SEQ ID NO: 291 - DNA, 20 bp, Artificial Sequence
  Internal forward primer
gtgtggaataggtatgttgg

SEQ ID NO: 292 - DNA, 20 bp, Artificial Sequence
  Internal forward primer
cacaggccaaacaggaaagg

SEQ ID NO: 293 - DNA, 20 bp, Artificial Sequence
  Internal forward primer
gactggcttcttctctttgc

SEQ ID NO: 294 - DNA, 20 bp, Artificial Sequence
  Internal forward primer
gaggtcaacctctaaagtgc

SEQ ID NO: 295 - DNA, 18 bp, Artificial Sequence
  Internal forward primer
cccttggcctggaaaagg

SEQ ID NO: 296 - DNA, 19 bp, Artificial Sequence
  Internal forward primer
gacacatgtaaactgtccc

SEQ ID NO: 297 - DNA, 19 bp, Artificial Sequence
  Internal forward primer
gtgtgcaaagggtttcagg

SEQ ID NO: 298 - DNA, 20 bp, Artificial Sequence
  Internal forward primer
gaacatacaagagggaatgg

SEQ ID NO: 299 - DNA, 20 bp, Artificial Sequence
  Internal forward primer
ctcaataccagaatcatcgc

SEQ ID NO: 300 - DNA, 19 bp, Artificial Sequence
  Internal forward primer
cagatgccagaagaatggg

SEQ ID NO: 301 - DNA, 20 bp, Artificial Sequence
  Internal forward primer
caacctaggctcaaaatgtg

SEQ ID NO: 302 - DNA, 19 bp, Artificial Sequence
  Internal forward primer
gtgcattaacgtggagcac

SEQ ID NO: 303 - DNA, 21 bp, Artificial Sequence
  Internal forward primer
gacatttagaaatggcctatc

SEQ ID NO: 304 - DNA, 21 bp, Artificial Sequence
  Internal forward primer
catgctgagtattgtacaaag

SEQ ID NO: 305 - DNA, 22 bp, Artificial Sequence
  Internal forward primer
gctatgttgatacatctaagac

SEQ ID NO: 306 - DNA, 21 bp, Artificial Sequence
  Internal forward primer
gttagaatgaatgactaagcc

SEQ ID NO: 307 - DNA, 20 bp, Artificial Sequence
  Internal forward primer
gagcatcagtcagttttagc

SEQ ID NO: 308 - DNA, 20 bp, Artificial Sequence
  Internal forward primer
ccagattctttacctgctac

SEQ ID NO: 309 - DNA, 20 bp, Artificial Sequence
  Internal forward primer
ctgcatcagctattctttcc

SEQ ID NO: 310 - DNA, 20 bp, Artificial Sequence
  Internal forward primer
cctttccacagactattgac

SEQ ID NO: 311 - DNA, 21 bp, Artificial Sequence
  Internal forward primer
gtctgtgaagataaaggaaag

SEQ ID NO: 312 - DNA, 21 bp, Artificial Sequence
  Internal forward primer
gatacaagatgtgaacattgg

SEQ ID NO: 313 - DNA, 19 bp, Artificial Sequence
  Internal forward primer
gtgattctgacccagtacc

SEQ ID NO: 314 - DNA, 19 bp, Artificial Sequence
  Internal forward primer
cttgactaggtggaagagc

SEQ ID NO: 315 - DNA, 19 bp, Artificial Sequence
  Internal forward primer
gttgaccatggcttagtcc

SEQ ID NO: 316 - DNA, 21 bp, Artificial Sequence
  Internal forward primer
gtcaattgcccagtgtttagg

SEQ ID NO: 317 - DNA, 19 bp, Artificial Sequence
  Internal forward primer
ggttttcagattggttggg

SEQ ID NO: 318 - DNA, 20 bp, Artificial Sequence
  Internal forward primer
caggatagacttggaaatgc

SEQ ID NO: 319 - DNA, 20 bp, Artificial Sequence
  Internal forward primer
cctacatctttcctgttagc

SEQ ID NO: 320 - DNA, 19 bp, Artificial Sequence
  Internal forward primer
ggtcttcatctttctccgg

SEQ ID NO: 321 - DNA, 19 bp, Artificial Sequence
  Internal forward primer
gttaggaggcatggatacc

SEQ ID NO: 322 - DNA, 19 bp, Artificial Sequence
  Internal forward primer
catatccccagttccttcc

SEQ ID NO: 323 - DNA, 22 bp, Artificial Sequence
  Internal forward primer
catgcataagatagtcaaaagc

SEQ ID NO: 324 - DNA, 19 bp, Artificial Sequence
  Internal forward primer
gctcaggccaagaaagacg

SEQ ID NO: 325 - DNA, 20 bp, Artificial Sequence
  Internal forward primer
gtctttgccaactcaacagg

SEQ ID NO: 326 - DNA, 21 bp, Artificial Sequence
  Internal forward primer
gagttcaatactttcttctcc

SEQ ID NO: 327 - DNA, 18 bp, Artificial Sequence
  Internal forward primer
ggacacaggaaggtgtgc

SEQ ID NO: 328 - DNA, 21 bp, Artificial Sequence
  Internal forward primer
gatacatgcaaagcaagaacc

SEQ ID NO: 329 - DNA, 20 bp, Artificial Sequence
  Internal forward primer
cttacatacttgggattggc

SEQ ID NO: 330 - DNA, 19 bp, Artificial Sequence
  Internal forward primer
gactgcttcaggacatggc

SEQ ID NO: 331 - DNA, 21 bp, Artificial Sequence
  Internal forward primer
ccattcttagtaacctatacc

SEQ ID NO: 332 - DNA, 19 bp, Artificial Sequence
  Internal forward primer
cttcctgagcaaagagacc

SEQ ID NO: 333 - DNA, 20 bp, Artificial Sequence
  Internal forward primer
caccattgtcatccagtacg

SEQ ID NO: 334 - DNA, 20 bp, Artificial Sequence
  Internal forward primer
ctggacaaagagtaatgtgc

SEQ ID NO: 335 - DNA, 19 bp, Artificial Sequence
  Internal forward primer
ctttgctcccaggtttggg

SEQ ID NO: 336 - DNA, 19 bp, Artificial Sequence
  Internal forward primer
ctccaaggctctgttctcc

SEQ ID NO: 337 - DNA, 20 bp, Artificial Sequence
  Internal forward primer
gtctgaagtaaagctcaacg

SEQ ID NO: 338 - DNA, 18 bp, Artificial Sequence
  Internal forward primer
gcagaacccaaggtcagc

SEQ ID NO: 339 - DNA, 19 bp, Artificial Sequence
  Internal forward primer
ccattctaccccacgaagg

SEQ ID NO: 340 - DNA, 20 bp, Artificial Sequence
  Internal forward primer
gaggatcctgaaacagaagc

SEQ ID NO: 341 - DNA, 19 bp, Artificial Sequence
  Internal forward primer
ctactggaatgctggcacg

SEQ ID NO: 342 - DNA, 18 bp, Artificial Sequence
  Internal forward primer
caccagagtcctccatgg

SEQ ID NO: 343 - DNA, 20 bp, Artificial Sequence
  Internal forward primer
ccacaaaagagaccataggg

SEQ ID NO: 344 - DNA, 19 bp, Artificial Sequence
  Internal forward primer
ctgtagcatagatcatggg

SEQ ID NO: 345 - DNA, 21 bp, Artificial Sequence
  Internal forward primer
ctttgttggctttccaattcg

SEQ ID NO: 346 - DNA, 19 bp, Artificial Sequence
  Internal forward primer
cgctcccttcctatgatcg

SEQ ID NO: 347 - DNA, 19 bp, Artificial Sequence
  Internal forward primer
ctcaccacaaacctcatgg

SEQ ID NO: 348 - DNA, 20 bp, Artificial Sequence
  Internal forward primer
gcagagcagaaatcactacc

SEQ ID NO: 349 - DNA, 19 bp, Artificial Sequence
  Internal forward primer
cttccacctcatgactagc

SEQ ID NO: 350 - DNA, 19 bp, Artificial Sequence
  Internal forward primer
caaagctttcctgtacacc

SEQ ID NO: 351 - DNA, 19 bp, Artificial Sequence
  Internal forward primer
caacacagcctgcatctcc

SEQ ID NO: 352 - DNA, 18 bp, Artificial Sequence
  Internal forward primer
ctcaaagctggggtaacg

SEQ ID NO: 353 - DNA, 19 bp, Artificial Sequence
  Internal forward primer
gagatactcctgagatggc

SEQ ID NO: 354 - DNA, 19 bp, Artificial Sequence
  Internal forward primer
gctcccaacaggcattacc

SEQ ID NO: 355 - DNA, 20 bp, Artificial Sequence
  Internal forward primer
cttgcttctttcacttagcc

SEQ ID NO: 356 - DNA, 19 bp, Artificial Sequence
  Internal forward primer
cagcaatgagtagctgacg

SEQ ID NO: 357 - DNA, 21 bp, Artificial Sequence
  Internal forward primer
caacttgcttttcacttaagg

SEQ ID NO: 358 - DNA, 21 bp, Artificial Sequence
  Internal forward primer
gatctacctaatgtttgaagc

SEQ ID NO: 359 - DNA, 20 bp, Artificial Sequence
  Internal forward primer
ggtgtagttgatttcactgg

SEQ ID NO: 360 - DNA, 19 bp, Artificial Sequence
  Internal forward primer
gagttgaggagtcgagagg

SEQ ID NO: 361 - DNA, 20 bp, Artificial Sequence
  Internal forward primer
ctgaggcttagagtttaggg

SEQ ID NO: 362 - DNA, 19 bp, Artificial Sequence
  Internal forward primer
cccaagccttttcagttcc

SEQ ID NO: 363 - DNA, 20 bp, Artificial Sequence
  Internal forward primer
ctagcaaagaatacgtgagc

SEQ ID NO: 364 - DNA, 21 bp, Artificial Sequence
  Internal forward primer
ggatatgttcaagtctcaacc

SEQ ID NO: 365 - DNA, 18 bp, Artificial Sequence
  Internal forward primer
gtctccgtgccctcaagg

SEQ ID NO: 366 - DNA, 20 bp, Artificial Sequence
  Internal forward primer
ctgtctcttttggtcctacc

SEQ ID NO: 367 - DNA, 19 bp, Artificial Sequence
  Internal forward primer
gcattacatgacggactgg

SEQ ID NO: 368 - DNA, 20 bp, Artificial Sequence
  Internal forward primer
ctccaatactgcagagatgg

SEQ ID NO: 369 - DNA, 18 bp, Artificial Sequence
  Internal forward primer
ggatgcacccagctaacc

SEQ ID NO: 370 - DNA, 20 bp, Artificial Sequence
  Internal forward primer
cctcatttgctgttaacacc

SEQ ID NO: 371 - DNA, 18 bp, Artificial Sequence
  Internal forward primer
cctcagcacagaggcagc

SEQ ID NO: 372 - DNA, 20 bp, Artificial Sequence
  Internal forward primer
gtggttaacagtctgactgg

SEQ ID NO: 373 - DNA, 19 bp, Artificial Sequence
  Internal forward primer
ggcagacccaattcttagc

SEQ ID NO: 374 - DNA, 20 bp, Artificial Sequence
  Internal forward primer
cctacagcaatactttgtcc

SEQ ID NO: 375 - DNA, 21 bp, Artificial Sequence
  Internal forward primer
ctgttctgttctacattcacc

SEQ ID NO: 376 - DNA, 19 bp, Artificial Sequence
  Internal forward primer
caagaacagagcccatggc

SEQ ID NO: 377 - DNA, 18 bp, Artificial Sequence
  Internal forward primer
ccagctccccatgaaggc

SEQ ID NO: 378 - DNA, 19 bp, Artificial Sequence
  Internal forward primer
gcaatagctcaggcaaacc

SEQ ID NO: 379 - DNA, 21 bp, Artificial Sequence
  Internal forward primer
gctcagaaacaaatcatttcc

SEQ ID NO: 380 - DNA, 21 bp, Artificial Sequence
  Internal forward primer
gcagactattcaaatgcttcc

SEQ ID NO: 381 - DNA, 19 bp, Artificial Sequence
  Internal forward primer
ctctgagaagcccatcagc

SEQ ID NO: 382 - DNA, 19 bp, Artificial Sequence
  Internal forward primer
gaaggatcaccatgaacgg

SEQ ID NO: 383 - DNA, 19 bp, Artificial Sequence
  Internal forward primer
gactatccagaaactgtgc

SEQ ID NO: 384 - DNA, 19 bp, Artificial Sequence
  Internal forward primer
ctgtgtctggtcttatggg

SEQ ID NO: 385 - DNA, 19 bp, Artificial Sequence
  Internal forward primer
gactgccaggaacgttagc

SEQ ID NO: 386 - DNA, 20 bp, Artificial Sequence
  Internal forward primer
cttgtcttgtcccttaaggg

SEQ ID NO: 387 - DNA, 18 bp, Artificial Sequence
  Internal forward primer
gacctgcctgggtgaacc

SEQ ID NO: 388 - DNA, 19 bp, Artificial Sequence
  Internal forward primer
gtggacccaactctgttgg

SEQ ID NO: 389 - DNA, 19 bp, Artificial Sequence
  Internal forward primer
ctttggctaagagggacgg

SEQ ID NO: 390 - DNA, 20 bp, Artificial Sequence
  Internal forward primer
cttgagatggaattctcacc

SEQ ID NO: 391 - DNA, 19 bp, Artificial Sequence
  Internal forward primer
gccctcccagaatcttagg

SEQ ID NO: 392 - DNA, 21 bp, Artificial Sequence
  Internal forward primer
gtaagagaatgagaattctcc

SEQ ID NO: 393 - DNA, 19 bp, Artificial Sequence
  Internal forward primer
ccaacaccaacagcgtagg

SEQ ID NO: 394 - DNA, 18 bp, Artificial Sequence
  Internal forward primer
cctggggaggagtacagg

SEQ ID NO: 395 - DNA, 20 bp, Artificial Sequence
  Internal forward primer
ccgaagttgctttctctagg

SEQ ID NO: 396 - DNA, 19 bp, Artificial Sequence
  Internal forward primer
gccaaggtacctttacagg

SEQ ID NO: 397 - DNA, 20 bp, Artificial Sequence
  Internal forward primer
cggacattagtctaaagtgg

SEQ ID NO: 398 - DNA, 20 bp, Artificial Sequence
  Internal forward primer
cacatttctttcctgtgtcc

SEQ ID NO: 399 - DNA, 19 bp, Artificial Sequence
  Internal forward primer
ctgtatggctcccaaaacc

SEQ ID NO: 400 - DNA, 18 bp, Artificial Sequence
  Internal forward primer
gaaggtcctgccatcagg

SEQ ID NO: 401 - DNA, 20 bp, Artificial Sequence
  Internal forward primer
gaacatttctccgtgattgc

SEQ ID NO: 402 - DNA, 18 bp, Artificial Sequence
  Internal forward primer
ggccttctgtctgtgacc

SEQ ID NO: 403 - DNA, 19 bp, Artificial Sequence
  Internal forward primer
catgccatcttcccctacc

SEQ ID NO: 404 - DNA, 20 bp, Artificial Sequence
  Internal forward primer
gcttgttgtagttactctgg

SEQ ID NO: 405 - DNA, 19 bp, Artificial Sequence
  Internal forward primer
gagttgacccagcgtttcc

SEQ ID NO: 406 - DNA, 20 bp, Artificial Sequence
  Internal forward primer
gtgacctttcttttcagtgc

SEQ ID NO: 407 - DNA, 20 bp, Artificial Sequence
  Internal forward primer
gttgtgactgtagtaagtgc

SEQ ID NO: 408 - DNA, 18 bp, Artificial Sequence
  Internal forward primer
gcagctgatgccgagagg

SEQ ID NO: 409 - DNA, 18 bp, Artificial Sequence
  Internal forward primer
ggatccaaccgtggaccc

SEQ ID NO: 410 - DNA, 18 bp, Artificial Sequence
  Internal forward primer
ccaatcccctccccaggg

SEQ ID NO: 411 - DNA, 20 bp, Artificial Sequence
  Internal forward primer
gcttgtagcatacataaggc

SEQ ID NO: 412 - DNA, 19 bp, Artificial Sequence
  Internal forward primer
caggttacggcaggagagg

SEQ ID NO: 413 - DNA, 18 bp, Artificial Sequence
  Internal forward primer
gtcagggttccagcatgc

SEQ ID NO: 414 - DNA, 19 bp, Artificial Sequence
  Internal forward primer
gttagacaggtggaagtcc

SEQ ID NO: 415 - DNA, 19 bp, Artificial Sequence
  Internal forward primer
ccatcaggctgtgatcagg

SEQ ID NO: 416 - DNA, 19 bp, Artificial Sequence
  Internal forward primer
cagcagacagtggaaacgg

SEQ ID NO: 417 - DNA, 19 bp, Artificial Sequence
  Internal forward primer
gcaactggtcagtctaagg

SEQ ID NO: 418 - DNA, 20 bp, Artificial Sequence
  Internal forward primer
ggacaaaaggaaacgtcagc

SEQ ID NO: 419 - DNA, 20 bp, Artificial Sequence
  Internal forward primer
gttgtgacttcagccatacc

SEQ ID NO: 420 - DNA, 19 bp, Artificial Sequence
  Internal forward primer
ggatacagcagaaaactgg

SEQ ID NO: 421 - DNA, 20 bp, Artificial Sequence
  Internal forward primer
ggtctcttgaaatcatcacc

SEQ ID NO: 422 - DNA, 19 bp, Artificial Sequence
  Internal forward primer
ctcttcaccagcaatacgg

SEQ ID NO: 423 - DNA, 19 bp, Artificial Sequence
  Internal forward primer
cttgggaatgctgagaacc

SEQ ID NO: 424 - DNA, 19 bp, Artificial Sequence
  Internal forward primer
gggtaacagatgccacagc

SEQ ID NO: 425 - DNA, 20 bp, Artificial Sequence
  Internal forward primer
ccttgcatgtcaccaaaagg

SEQ ID NO: 426 - DNA, 19 bp, Artificial Sequence
  Internal forward primer
gtcctctgctaacctgtcc

SEQ ID NO: 427 - DNA, 19 bp, Artificial Sequence
  Internal forward primer
ctgctagtctgaagactcc

SEQ ID NO: 428 - DNA, 20 bp, Artificial Sequence
  Internal forward primer
gtcgcaacaataccacaagg

SEQ ID NO: 429 - DNA, 20 bp, Artificial Sequence
  Internal forward primer
ctctcacttttgaccagacc

SEQ ID NO: 430 - DNA, 20 bp, Artificial Sequence
  Internal forward primer
ctgtcctaaggaatccaacc

SEQ ID NO: 431 - DNA, 20 bp, Artificial Sequence
  Internal forward primer
ctttctgtctcatctgaagg

SEQ ID NO: 432 - DNA, 20 bp, Artificial Sequence
  Internal forward primer
gctgtgtactgcaaagatgg

SEQ ID NO: 433 - DNA, 19 bp, Artificial Sequence
  Internal forward primer
gtgctcaagccacaatacc

SEQ ID NO: 434 - DNA, 20 bp, Artificial Sequence
  Internal forward primer
gcgtttgtttgcttgaaagc

SEQ ID NO: 435 - DNA, 19 bp, Artificial Sequence
  Internal forward primer
cactcatttgctccgttgc

SEQ ID NO: 436 - DNA, 20 bp, Artificial Sequence
  Internal forward primer
ctggggaaacagacacaagc

SEQ ID NO: 437 - DNA, 20 bp, Artificial Sequence
  Internal forward primer
gggaaatgtaaagtctgagg

SEQ ID NO: 438 - DNA, 20 bp, Artificial Sequence
  Internal forward primer
catacatcttcagccaaggc

SEQ ID NO: 439 - DNA, 19 bp, Artificial Sequence
  Internal forward primer
ctctgtgtcacgtagtagc

SEQ ID NO: 440 - DNA, 19 bp, Artificial Sequence
  Internal forward primer
ctgagttgccatgcattcg

SEQ ID NO: 441 - DNA, 19 bp, Artificial Sequence
  Internal forward primer
gagctaggagaggtacagg

SEQ ID NO: 442 - DNA, 18 bp, Artificial Sequence
  Internal forward primer
ctggtgctgagactctgg

SEQ ID NO: 443 - DNA, 20 bp, Artificial Sequence
  Internal forward primer
ctgatgagccttagaattgg

SEQ ID NO: 444 - DNA, 19 bp, Artificial Sequence
  Internal forward primer
ggaagattctggagatacc

SEQ ID NO: 445 - DNA, 19 bp, Artificial Sequence
  Internal forward primer
caagcttctgaagctacgc

SEQ ID NO: 446 - DNA, 20 bp, Artificial Sequence
  Internal forward primer
ggttacaattcatcccaccc

SEQ ID NO: 447 - DNA, 20 bp, Artificial Sequence
  Internal forward primer
cccatagctattgaaatgcc

SEQ ID NO: 448 - DNA, 20 bp, Artificial Sequence
  Internal forward primer
ggaattcagacctcataggg

SEQ ID NO: 449 - DNA, 21 bp, Artificial Sequence
  Internal forward primer
cattcctaatgtttcaggtgg

SEQ ID NO: 450 - DNA, 20 bp, Artificial Sequence
  Internal forward primer
gtgcttgactttggaaaccc

SEQ ID NO: 451 - DNA, 20 bp, Artificial Sequence
  Internal forward primer
gctttctggctttgtcaagc

SEQ ID NO: 452 - DNA, 20 bp, Artificial Sequence
  Internal forward primer
gagtttcagagcttctctag

SEQ ID NO: 453 - DNA, 18 bp, Artificial Sequence
  Internal forward primer
catggtggacgtggatgc

SEQ ID NO: 454 - DNA, 19 bp, Artificial Sequence
  Internal forward primer
gaaaccatggatgcacacc

SEQ ID NO: 455 - DNA, 18 bp, Artificial Sequence
  Internal forward primer
gctttgaggtggcgatcg

SEQ ID NO: 456 - DNA, 19 bp, Artificial Sequence
  Internal forward primer
ctactcctggaagctcacc

SEQ ID NO: 457 - DNA, 19 bp, Artificial Sequence
  Internal forward primer
cggtatccatggtccaacc

SEQ ID NO: 458 - DNA, 19 bp, Artificial Sequence
  Internal forward primer
gcaaaaccgagtgttctcc

SEQ ID NO: 459 - DNA, 19 bp, Artificial Sequence
  Internal forward primer
caggccaagatatgaaggc

SEQ ID NO: 460 - DNA, 19 bp, Artificial Sequence
  Internal forward primer
gcaaacatgggagccaagc

SEQ ID NO: 461 - DNA, 19 bp, Artificial Sequence
  Internal forward primer
ccagatcccagagtaaagg

SEQ ID NO: 462 - DNA, 20 bp, Artificial Sequence
  Internal forward primer
gcagaaaggaagaaggttcc

SEQ ID NO: 463 - DNA, 19 bp, Artificial Sequence
  Internal forward primer
ctggagaaactaggaaggc

SEQ ID NO: 464 - DNA, 18 bp, Artificial Sequence
  Internal forward primer
caggggacacgcattagc

SEQ ID NO: 465 - DNA, 20 bp, Artificial Sequence
  Internal forward primer
gtggacggattcaatgatcc

SEQ ID NO: 466 - DNA, 19 bp, Artificial Sequence
  Internal forward primer
gaggacactcccattctgg

SEQ ID NO: 467 - DNA, 20 bp, Artificial Sequence
  Internal forward primer
ctgaatgaggccacttttcc

SEQ ID NO: 468 - DNA, 20 bp, Artificial Sequence
  Internal forward primer
gtaacttcctggttcttgcc

SEQ ID NO: 469 - DNA, 20 bp, Artificial Sequence
  Internal forward primer
cattgctgatgcatgagtgc

SEQ ID NO: 470 - DNA, 19 bp, Artificial Sequence
  Internal forward primer
gcatggtgaatgcagaacg

SEQ ID NO: 471 - DNA, 20 bp, Artificial Sequence
  Internal forward primer
caactgtgtaaacctttgcc

SEQ ID NO: 472 - DNA, 19 bp, Artificial Sequence
  Internal forward primer
cacatagcagcacagaagc

SEQ ID NO: 473 - DNA, 20 bp, Artificial Sequence
  Internal forward primer
ctgtgttctgcacatactgc

SEQ ID NO: 474 - DNA, 20 bp, Artificial Sequence
  Internal forward primer
ccacttgtctggtattcacc

SEQ ID NO: 475 - DNA, 19 bp, Artificial Sequence
  Internal forward primer
cctactcagctcttgttcc

SEQ ID NO: 476 - DNA, 19 bp, Artificial Sequence
  Internal forward primer
cacactttactcaggttgg

SEQ ID NO: 477 - DNA, 21 bp, Artificial Sequence
  Internal forward primer
gaagcaatggaaagatttggg

SEQ ID NO: 478 - DNA, 19 bp, Artificial Sequence
  Internal forward primer
ctttggtgctaaagcttcc

SEQ ID NO: 479 - DNA, 19 bp, Artificial Sequence
  Internal forward primer
cccacaaaggtctttcagg

SEQ ID NO: 480 - DNA, 19 bp, Artificial Sequence
  Internal forward primer
ctacaggagggatcagagc

SEQ ID NO: 481 - DNA, 18 bp, Artificial Sequence
  Internal forward primer
ctccactggggacggtcc

SEQ ID NO: 482 - DNA, 20 bp, Artificial Sequence
  Internal forward primer
gcaacactgtgaaaagatgc

SEQ ID NO: 483 - DNA, 20 bp, Artificial Sequence
  Internal forward primer
ctttaggactggaggaatgg

SEQ ID NO: 484 - DNA, 20 bp, Artificial Sequence
  Internal forward primer
gtttctctttcagagctacc

SEQ ID NO: 485 - DNA, 20 bp, Artificial Sequence
  Internal forward primer
gttctgagaagcagatgagc

SEQ ID NO: 486 - DNA, 19 bp, Artificial Sequence
  Internal forward primer
gctaaagtggaatgagagg

SEQ ID NO: 487 - DNA, 19 bp, Artificial Sequence
  Internal forward primer
cgcttaccggaaacaaacc

SEQ ID NO: 488 - DNA, 20 bp, Artificial Sequence
  Internal forward primer
gttacatcatgtcagatggc

SEQ ID NO: 489 - DNA, 21 bp, Artificial Sequence
  Internal forward primer
ggaaaagattctttcctttgc

SEQ ID NO: 490 - DNA, 20 bp, Artificial Sequence
  Internal forward primer
cagtctgtcaactctttagg

SEQ ID NO: 491 - DNA, 20 bp, Artificial Sequence
  Internal forward primer
ccagttgtgtttctgttccc

SEQ ID NO: 492 - DNA, 21 bp, Artificial Sequence
  Internal forward primer
gaagtggtaaagtttcttcgc

SEQ ID NO: 493 - DNA, 19 bp, Artificial Sequence
  Internal forward primer
ctgttcttctctgggctgg

SEQ ID NO: 494 - DNA, 19 bp, Artificial Sequence
  Internal forward primer
ccatgaatgcggaggaagc

SEQ ID NO: 495 - DNA, 20 bp, Artificial Sequence
  Internal forward primer
cagctgagtaacaaacatcc

SEQ ID NO: 496 - DNA, 19 bp, Artificial Sequence
  Internal forward primer
ctcagaggtccaagaaagc

SEQ ID NO: 497 - DNA, 20 bp, Artificial Sequence
  Internal forward primer
gctcttggaagaactttagg

SEQ ID NO: 498 - DNA, 20 bp, Artificial Sequence
  Internal forward primer
gtttcactctggctaacagg

SEQ ID NO: 499 - DNA, 20 bp, Artificial Sequence
  Internal forward primer
gaacagctgtttggacatgg

SEQ ID NO: 500 - DNA, 19 bp, Artificial Sequence
  Internal forward primer
gtcaacgcaagggtaatcc

SEQ ID NO: 501 - DNA, 19 bp, Artificial Sequence
  Internal forward primer
ggagaggaagtgtcacagg

SEQ ID NO: 502 - DNA, 18 bp, Artificial Sequence
  Internal forward primer
ggactgccagaggcttgg

SEQ ID NO: 503 - DNA, 20 bp, Artificial Sequence
  Internal forward primer
gcttgacaacagcaacaacc

SEQ ID NO: 504 - DNA, 19 bp, Artificial Sequence
  Internal forward primer
gcgatgaaaccagtatccc

SEQ ID NO: 505 - DNA, 19 bp, Artificial Sequence
  Internal forward primer
ctcagaacggaacgtgacc

SEQ ID NO: 506 - DNA, 20 bp, Artificial Sequence
  Internal forward primer
gcagatatgggtggaaatgg

SEQ ID NO: 507 - DNA, 21 bp, Artificial Sequence
  Internal forward primer
gaaagaacaggatgagaatgc

SEQ ID NO: 508 - DNA, 18 bp, Artificial Sequence
  Internal forward primer
ctccagactgggtaccgc

SEQ ID NO: 509 - DNA, 18 bp, Artificial Sequence
  Internal forward primer
cgaaaagccgaggacagc

SEQ ID NO: 510 - DNA, 18 bp, Artificial Sequence
  Internal forward primer
ccattggggacctcttgg

SEQ ID NO: 511 - DNA, 18 bp, Artificial Sequence
  Internal forward primer
gcagaggccatgcatagg

SEQ ID NO: 512 - DNA, 20 bp, Artificial Sequence
  Internal forward primer
ccattacatatccacactgg

SEQ ID NO: 513 - DNA, 19 bp, Artificial Sequence
  Internal forward primer
gagacttgggatcttaccg

SEQ ID NO: 514 - DNA, 19 bp, Artificial Sequence
  Internal forward primer
cagactctgctttaggagg

SEQ ID NO: 515 - DNA, 20 bp, Artificial Sequence
  Internal forward primer
gttgcactgtactatacagg

SEQ ID NO: 516 - DNA, 18 bp, Artificial Sequence
  Internal forward primer
cactggagtgccttctgg

SEQ ID NO: 517 - DNA, 19 bp, Artificial Sequence
  Internal forward primer
caacccacttttcatcagc

SEQ ID NO: 518 - DNA, 21 bp, Artificial Sequence
  Internal forward primer
caagtgcatggtgagatatgg

SEQ ID NO: 519 - DNA, 20 bp, Artificial Sequence
  Internal forward primer
ctgatgaggcaatacattgg

SEQ ID NO: 520 - DNA, 21 bp, Artificial Sequence
  Internal forward primer
gagttctctgaaatgattagc

SEQ ID NO: 521 - DNA, 18 bp, Artificial Sequence
  Internal forward primer
cagctgtgtctcaagagg

SEQ ID NO: 522 - DNA, 20 bp, Artificial Sequence
  Internal forward primer
cagtctgaggaggaaagagg

SEQ ID NO: 523 - DNA, 20 bp, Artificial Sequence
  Internal forward primer
gtccattcttgtcctgaagg

SEQ ID NO: 524 - DNA, 20 bp, Artificial Sequence
  Internal forward primer
cagaattcaagcaactcagg

SEQ ID NO: 525 - DNA, 20 bp, Artificial Sequence
  Internal forward primer
gagttcaaacctcggtttgg

SEQ ID NO: 526 - DNA, 19 bp, Artificial Sequence
  Internal forward primer
caaaccctgatgggtttgc

SEQ ID NO: 527 - DNA, 20 bp, Artificial Sequence
  Internal forward primer
gttttggaggtatggcaacc

SEQ ID NO: 528 - DNA, 20 bp, Artificial Sequence
  Internal forward primer
gcacaacatgaagaaatgcc

SEQ ID NO: 529 - DNA, 20 bp, Artificial Sequence
  Internal forward primer
cttttgcaaggaaatcaggg

SEQ ID NO: 530 - DNA, 19 bp, Artificial Sequence
  Internal forward primer
gtctggtggtaacagtacc

SEQ ID NO: 531 - DNA, 20 bp, Artificial Sequence
  Internal forward primer
gacaagacacatgtaaaccc

SEQ ID NO: 532 - DNA, 18 bp, Artificial Sequence
  Internal forward primer
gcctatgcagtgcgaggc

SEQ ID NO: 533 - DNA, 19 bp, Artificial Sequence
  Internal forward primer
gtttgtgagcactccatcc

SEQ ID NO: 534 - DNA, 19 bp, Artificial Sequence
  Internal forward primer
gcaaagaatggtgcgatcg

SEQ ID NO: 535 - DNA, 21 bp, Artificial Sequence
  Internal forward primer
ccttcatctgacatagttagc

SEQ ID NO: 536 - DNA, 20 bp, Artificial Sequence
  Internal forward primer
cctttcctattctcaatggc

SEQ ID NO: 537 - DNA, 20 bp, Artificial Sequence
  Internal forward primer
cttctgaaggaagtcatccg

SEQ ID NO: 538 - DNA, 19 bp, Artificial Sequence
  Internal forward primer
gccaagaggtaatcttcgg

SEQ ID NO: 539 - DNA, 20 bp, Artificial Sequence
  Internal forward primer
ggtgaggtgtatagagatcc

SEQ ID NO: 540 - DNA, 18 bp, Artificial Sequence
  Internal forward primer
gcagaagagggctcttgg

SEQ ID NO: 541 - DNA, 19 bp, Artificial Sequence
  Internal forward primer
ctgctcctcaattcagtcc

SEQ ID NO: 542 - DNA, 20 bp, Artificial Sequence
  Internal forward primer
gaagagaaaaggccatctgc

SEQ ID NO: 543 - DNA, 19 bp, Artificial Sequence
  Internal forward primer
gacctagaggacaggaacc

SEQ ID NO: 544 - DNA, 21 bp, Artificial Sequence
  Internal forward primer
gcttttcacaattctgagtcc

SEQ ID NO: 545 - DNA, 20 bp, Artificial Sequence
  Internal forward primer
ccagtaaagactcactcagc

SEQ ID NO: 546 - DNA, 21 bp, Artificial Sequence
  Internal forward primer
cttcattctaatgagaagtcc

SEQ ID NO: 547 - DNA, 19 bp, Artificial Sequence
  Internal forward primer
cagctttacagatgagacg

SEQ ID NO: 548 - DNA, 20 bp, Artificial Sequence
  Internal forward primer
caacttcgaagagaaagtcc

SEQ ID NO: 549 - DNA, 19 bp, Artificial Sequence
  Internal forward primer
gaatcctcaccaacagtcg

SEQ ID NO: 550 - DNA, 18 bp, Artificial Sequence
  Internal forward primer
caaacctcccaggtcacc

SEQ ID NO: 551 - DNA, 18 bp, Artificial Sequence
  Internal forward primer
cacgcaacgggtgcttcc

SEQ ID NO: 552 - DNA, 19 bp, Artificial Sequence
  Internal forward primer
catgtgagggaaggaatcg

SEQ ID NO: 553 - DNA, 20 bp, Artificial Sequence
  Internal forward primer
cacacttatgacaagtgagc

SEQ ID NO: 554 - DNA, 21 bp, Artificial Sequence
  Internal forward primer
ctcaggtttgttttgttaagg

SEQ ID NO: 555 - DNA, 19 bp, Artificial Sequence
  Internal forward primer
ccatagaggggtccattgc

SEQ ID NO: 556 - DNA, 20 bp, Artificial Sequence
  Internal forward primer
gtgctagtagggtctttagc

SEQ ID NO: 557 - DNA, 19 bp, Artificial Sequence
  Internal forward primer
ccaagtgaacatgcactcc

SEQ ID NO: 558 - DNA, 20 bp, Artificial Sequence
  Internal forward primer
caggagactgcagtatcagg

SEQ ID NO: 559 - DNA, 19 bp, Artificial Sequence
  Internal forward primer
catgcctcaaccttcttcc

SEQ ID NO: 560 - DNA, 20 bp, Artificial Sequence
  Internal forward primer
ggaacatttcagttgactgg

SEQ ID NO: 561 - DNA, 18 bp, Artificial Sequence
  Internal forward primer
ctaggattgccactgggc

SEQ ID NO: 562 - DNA, 19 bp, Artificial Sequence
  Internal forward primer
gctgattaggtagtatgcc

SEQ ID NO: 563 - DNA, 19 bp, Artificial Sequence
  Internal forward primer
gtatcacactcctcagagg

SEQ ID NO: 564 - DNA, 19 bp, Artificial Sequence
  Internal forward primer
gtgcagtccttacaaaagg

SEQ ID NO: 565 - DNA, 20 bp, Artificial Sequence
  Internal forward primer
cctattccacagaaaggatg

SEQ ID NO: 566 - DNA, 20 bp, Artificial Sequence
  Internal forward primer
cagtgatgaacaacagtctc

SEQ ID NO: 567 - DNA, 20 bp, Artificial Sequence
  Internal forward primer
ctatcattctgggacttctg

SEQ ID NO: 568 - DNA, 20 bp, Artificial Sequence
  Internal forward primer
cccaatctagaggtggaaag

SEQ ID NO: 569 - DNA, 20 bp, Artificial Sequence
  Internal forward primer
ctacctttcttagcctttcc

SEQ ID NO: 570 - DNA, 21 bp, Artificial Sequence
  Internal forward primer
gtgagaaatgtttgagtgatg

SEQ ID NO: 571 - DNA, 19 bp, Artificial Sequence
  Internal forward primer
gctatcagggtcaaccaag

SEQ ID NO: 572 - DNA, 20 bp, Artificial Sequence
  Internal forward primer
cagcaccctgatcaataagg

SEQ ID NO: 573 - DNA, 21 bp, Artificial Sequence
  Internal forward primer
ctgaaactctaatagacaagc

SEQ ID NO: 574 - DNA, 19 bp, Artificial Sequence
  Internal forward primer
cagttgggtcaatggtcag

SEQ ID NO: 575 - DNA, 20 bp, Artificial Sequence
  Internal forward primer
gtatctccttctttcttggc

SEQ ID NO: 576 - DNA, 20 bp, Artificial Sequence
  Internal forward primer
cagttcatgatagcttgctg

SEQ ID NO: 577 - DNA, 19 bp, Artificial Sequence
  Reverse primer
catgtggcaggcacatacg

SEQ ID NO: 578 - DNA, 19 bp, Artificial Sequence
  Reverse primer
ctgacctcagagctcatgg

SEQ ID NO: 579 - DNA, 20 bp, Artificial Sequence
  Reverse primer
gagaacttgcatccatttgc

SEQ ID NO: 580 - DNA, 19 bp, Artificial Sequence
  Reverse primer
gcctattgctttgaggagc

SEQ ID NO: 581 - DNA, 20 bp, Artificial Sequence
  Reverse primer
cacaacaggtgtttgagagc

SEQ ID NO: 582 - DNA, 19 bp, Artificial Sequence
  Reverse primer
gagtgctccattcactacc

SEQ ID NO: 583 - DNA, 20 bp, Artificial Sequence
  Reverse primer
cagcaccccaaatctgatcc

SEQ ID NO: 584 - DNA, 18 bp, Artificial Sequence
  Reverse primer
ggtgtttccccactagcc

SEQ ID NO: 585 - DNA, 20 bp, Artificial Sequence
  Reverse primer
cttcttgtattcttgtgagg

SEQ ID NO: 586 - DNA, 19 bp, Artificial Sequence
  Reverse primer
gaaaggctgtcctgaaacg

SEQ ID NO: 587 - DNA, 20 bp, Artificial Sequence
  Reverse primer
ccagttgaggaaaccaaagc

SEQ ID NO: 588 - DNA, 19 bp, Artificial Sequence
  Reverse primer
ctgctagaggagacactgc

SEQ ID NO: 589 - DNA, 19 bp, Artificial Sequence
  Reverse primer
gaacctgctggaactgaag

SEQ ID NO: 590 - DNA, 21 bp, Artificial Sequence
  Reverse primer
ctcatagaacttattgtgctg

SEQ ID NO: 591 - DNA, 21 bp, Artificial Sequence
  Reverse primer
ccaatatccctaaatctcatc

SEQ ID NO: 592 - DNA, 20 bp, Artificial Sequence
  Reverse primer
catagccttctgtatgttcc

SEQ ID NO: 593 - DNA, 21 bp, Artificial Sequence
  Reverse primer
ctttgttgcttttgtaatggg

SEQ ID NO: 594 - DNA, 20 bp, Artificial Sequence
  Reverse primer
caggatttagtggctgatag

SEQ ID NO: 595 - DNA, 21 bp, Artificial Sequence
  Reverse primer
gggaatttctataagatgcag

SEQ ID NO: 596 - DNA, 21 bp, Artificial Sequence
  Reverse primer
cacaattcctatcaaagcttg

SEQ ID NO: 597 - DNA, 20 bp, Artificial Sequence
  Reverse primer
gagtcagaaaaccatgactc

SEQ ID NO: 598 - DNA, 20 bp, Artificial Sequence
  Reverse primer
ggttcaaagcgaagactatc

SEQ ID NO: 599 - DNA, 21 bp, Artificial Sequence
  Reverse primer
ggattagacctatttgttgag

SEQ ID NO: 600 - DNA, 21 bp, Artificial Sequence
  Reverse primer
cggaacaattacaagtaaagc

SEQ ID NO: 601 - DNA, 20 bp, Artificial Sequence
  Reverse primer
catgaggctaagaaaacagc

SEQ ID NO: 602 - DNA, 19 bp, Artificial Sequence
  Reverse primer
ccaaggggatcaagcaagc

SEQ ID NO: 603 - DNA, 19 bp, Artificial Sequence
  Reverse primer
gagacagacagtctcaacg

SEQ ID NO: 604 - DNA, 18 bp, Artificial Sequence
  Reverse primer
ggggtccccagactgtgg

SEQ ID NO: 605 - DNA, 19 bp, Artificial Sequence
  Reverse primer
gctgtaagtggaccatggc

SEQ ID NO: 606 - DNA, 19 bp, Artificial Sequence
  Reverse primer
ctacagctggttcctgtcg

SEQ ID NO: 607 - DNA, 19 bp, Artificial Sequence
  Reverse primer
cactgtagcagtagagcgc

SEQ ID NO: 608 - DNA, 22 bp, Artificial Sequence
  Reverse primer
gactttgctttacaatctttgg

SEQ ID NO: 609 - DNA, 19 bp, Artificial Sequence
  Reverse primer
gattgggtcgattgactcc

SEQ ID NO: 610 - DNA, 19 bp, Artificial Sequence
  Reverse primer
cttcacatgaacgcctacc

SEQ ID NO: 611 - DNA, 20 bp, Artificial Sequence
  Reverse primer
gctttactttactttgtccc

SEQ ID NO: 612 - DNA, 19 bp, Artificial Sequence
  Reverse primer
gtaactgtggagtggatgg

SEQ ID NO: 613 - DNA, 20 bp, Artificial Sequence
  Reverse primer
ctcctcctatgcttctgacc

SEQ ID NO: 614 - DNA, 19 bp, Artificial Sequence
  Reverse primer
ctcaggtggactatgatcc

SEQ ID NO: 615 - DNA, 20 bp, Artificial Sequence
  Reverse primer
ctcaatgggtagagaaatcc

SEQ ID NO: 616 - DNA, 19 bp, Artificial Sequence
  Reverse primer
ggacttggccatgagttgg

SEQ ID NO: 617 - DNA, 19 bp, Artificial Sequence
  Reverse primer
gctctttgtactcttgagc

SEQ ID NO: 618 - DNA, 20 bp, Artificial Sequence
  Reverse primer
ctacctgctagttgatgtgg

SEQ ID NO: 619 - DNA, 19 bp, Artificial Sequence
  Reverse primer
cttctcccaattcccatgg

SEQ ID NO: 620 - DNA, 20 bp, Artificial Sequence
  Reverse primer
catagatgtcagaagtctcg

SEQ ID NO: 621 - DNA, 19 bp, Artificial Sequence
  Reverse primer
gcttggggaaagccaaagg

SEQ ID NO: 622 - DNA, 19 bp, Artificial Sequence
  Reverse primer
ccactgtctaacttgttcc

SEQ ID NO: 623 - DNA, 20 bp, Artificial Sequence
  Reverse primer
cttcagtcatctgtgatacc

SEQ ID NO: 624 - DNA, 19 bp, Artificial Sequence
  Reverse primer
gtggagcacagcacatacc

SEQ ID NO: 625 - DNA, 19 bp, Artificial Sequence
  Reverse primer
cttgatttgtcagggtggg

SEQ ID NO: 626 - DNA, 19 bp, Artificial Sequence
  Reverse primer
gggaatgaatctgcaaccc

SEQ ID NO: 627 - DNA, 19 bp, Artificial Sequence
  Reverse primer
catcctgggctatgagacg

SEQ ID NO: 628 - DNA, 20 bp, Artificial Sequence
  Reverse primer
gtgaatggaatgagcattgg

SEQ ID NO: 629 - DNA, 19 bp, Artificial Sequence
  Reverse primer
gctgaccttgaccatcacc

SEQ ID NO: 630 - DNA, 19 bp, Artificial Sequence
  Reverse primer
gaagggtttgggattctgg

SEQ ID NO: 631 - DNA, 22 bp, Artificial Sequence
  Reverse primer
gaagctgtcaaatgactaatgc

SEQ ID NO: 632 - DNA, 19 bp, Artificial Sequence
  Reverse primer
gcgaatgcagagaaacagc

SEQ ID NO: 633 - DNA, 19 bp, Artificial Sequence
  Reverse primer
ggcattgcagatatgtgcc

SEQ ID NO: 634 - DNA, 20 bp, Artificial Sequence
  Reverse primer
caagcactgtttgttcaagg

SEQ ID NO: 635 - DNA, 20 bp, Artificial Sequence
  Reverse primer
ggaaacaacaggatcatagg

SEQ ID NO: 636 - DNA, 20 bp, Artificial Sequence
  Reverse primer
ggagattgctaatgatttgc

SEQ ID NO: 637 - DNA, 19 bp, Artificial Sequence
  Reverse primer
gagacagtgaccagatcgg

SEQ ID NO: 638 - DNA, 19 bp, Artificial Sequence
  Reverse primer
cacctctcagtggataggc

SEQ ID NO: 639 - DNA, 19 bp, Artificial Sequence
  Reverse primer
gacctcaagtcatggtagg

SEQ ID NO: 640 - DNA, 21 bp, Artificial Sequence
  Reverse primer
ggtttgaagaacttaccaagc

SEQ ID NO: 641 - DNA, 20 bp, Artificial Sequence
  Reverse primer
cagacattactaaagaacgc

SEQ ID NO: 642 - DNA, 21 bp, Artificial Sequence
  Reverse primer
cttcagaattcttcaacatgg

SEQ ID NO: 643 - DNA, 19 bp, Artificial Sequence
  Reverse primer
ggttggagaagtgtgatcc

SEQ ID NO: 644 - DNA, 20 bp, Artificial Sequence
  Reverse primer
gaaccactttggagacttgg

SEQ ID NO: 645 - DNA, 20 bp, Artificial Sequence
  Reverse primer
ctagagttggtgacaattgc

SEQ ID NO: 646 - DNA, 19 bp, Artificial Sequence
  Reverse primer
gagcaaatgtcacctcacg

SEQ ID NO: 647 - DNA, 20 bp, Artificial Sequence
  Reverse primer
gaagtgaggataagtgaacc

SEQ ID NO: 648 - DNA, 21 bp, Artificial Sequence
  Reverse primer
gttgacaaggaagacaaaagg

SEQ ID NO: 649 - DNA, 20 bp, Artificial Sequence
  Reverse primer
ctcctctattgccagaatgc

SEQ ID NO: 650 - DNA, 20 bp, Artificial Sequence
  Reverse primer
gaataccaaacagacttagc

SEQ ID NO: 651 - DNA, 19 bp, Artificial Sequence
  Reverse primer
gtttcctgaaggcctctgg

SEQ ID NO: 652 - DNA, 18 bp, Artificial Sequence
  Reverse primer
gcatgccaggtgaaggcc

SEQ ID NO: 653 - DNA, 20 bp, Artificial Sequence
  Reverse primer
cagtttagaagtaggagtgc

SEQ ID NO: 654 - DNA, 19 bp, Artificial Sequence
  Reverse primer
ctggaggtccaatcaaagg

SEQ ID NO: 655 - DNA, 20 bp, Artificial Sequence
  Reverse primer
gtgcagtttgcaagaaaggc

SEQ ID NO: 656 - DNA, 19 bp, Artificial Sequence
  Reverse primer
cggctgtcctttctttggg

SEQ ID NO: 657 - DNA, 20 bp, Artificial Sequence
  Reverse primer
cctttccttaggataacagc

SEQ ID NO: 658 - DNA, 19 bp, Artificial Sequence
  Reverse primer
ggacaggaatagaaatgcc

SEQ ID NO: 659 - DNA, 19 bp, Artificial Sequence
  Reverse primer
ccagtcttatgcattgtgc

SEQ ID NO: 660 - DNA, 20 bp, Artificial Sequence
  Reverse primer
gaaactaagcacgtgcatcc

SEQ ID NO: 661 - DNA, 20 bp, Artificial Sequence
  Reverse primer
cagtctttggtagacgatgg

SEQ ID NO: 662 - DNA, 18 bp, Artificial Sequence
  Reverse primer
gtagccacaggtggcacc

SEQ ID NO: 663 - DNA, 19 bp, Artificial Sequence
  Reverse primer
cttccattctgtagggagg

SEQ ID NO: 664 - DNA, 20 bp, Artificial Sequence
  Reverse primer
gctcatttcctgtaaacagc

SEQ ID NO: 665 - DNA, 19 bp, Artificial Sequence
  Reverse primer
ggagactatgcatctttcc

SEQ ID NO: 666 - DNA, 18 bp, Artificial Sequence
  Reverse primer
ctggtcagtgggcagccg

SEQ ID NO: 667 - DNA, 18 bp, Artificial Sequence
  Reverse primer
ggggtgtacagtaaacgg

SEQ ID NO: 668 - DNA, 19 bp, Artificial Sequence
  Reverse primer
cagatgcatgactatgggg

SEQ ID NO: 669 - DNA, 20 bp, Artificial Sequence
  Reverse primer
ggctagattcatccacttgc

SEQ ID NO: 670 - DNA, 21 bp, Artificial Sequence
  Reverse primer
caaagatttggaactctgtgc

SEQ ID NO: 671 - DNA, 19 bp, Artificial Sequence
  Reverse primer
gctcagagcacatggttcc

SEQ ID NO: 672 - DNA, 19 bp, Artificial Sequence
  Reverse primer
ctttgtcccctgaggtagc

SEQ ID NO: 673 - DNA, 19 bp, Artificial Sequence
  Reverse primer
ccacttttggacaagtgcc

SEQ ID NO: 674 - DNA, 20 bp, Artificial Sequence
  Reverse primer
cagccatcactatctattgc

SEQ ID NO: 675 - DNA, 19 bp, Artificial Sequence
  Reverse primer
catgtgatcgccagaatcg

SEQ ID NO: 676 - DNA, 19 bp, Artificial Sequence
  Reverse primer
ctcttacctctcggatacc

SEQ ID NO: 677 - DNA, 21 bp, Artificial Sequence
  Reverse primer
cctttcttgatgattctctgg

SEQ ID NO: 678 - DNA, 19 bp, Artificial Sequence
  Reverse primer
gaactgggctggtctttcc

SEQ ID NO: 679 - DNA, 21 bp, Artificial Sequence
  Reverse primer
ctactgtttctgtgatcaacc

SEQ ID NO: 680 - DNA, 20 bp, Artificial Sequence
  Reverse primer
cgagtatcccatttctaagc

SEQ ID NO: 681 - DNA, 20 bp, Artificial Sequence
  Reverse primer
ggaaagtccttgaaagaagg

SEQ ID NO: 682 - DNA, 20 bp, Artificial Sequence
  Reverse primer
ggtaatctgcttttctaagg

SEQ ID NO: 683 - DNA, 19 bp, Artificial Sequence
  Reverse primer
ctccaaaagggtcctgtgg

SEQ ID NO: 684 - DNA, 19 bp, Artificial Sequence
  Reverse primer
ccacttatccctaaggagc

SEQ ID NO: 685 - DNA, 19 bp, Artificial Sequence
  Reverse primer
gtgtgaaatggatgaggcg

SEQ ID NO: 686 - DNA, 20 bp, Artificial Sequence
  Reverse primer
cacttacaggcctaactagg

SEQ ID NO: 687 - DNA, 18 bp, Artificial Sequence
  Reverse primer
ccaactggctgctagagc

SEQ ID NO: 688 - DNA, 19 bp, Artificial Sequence
  Reverse primer
gagctagcatgcattcagg

SEQ ID NO: 689 - DNA, 21 bp, Artificial Sequence
  Reverse primer
ctcaagttgtcaaatcagtgg

SEQ ID NO: 690 - DNA, 20 bp, Artificial Sequence
  Reverse primer
gaacttggtgcttctatggc

SEQ ID NO: 691 - DNA, 19 bp, Artificial Sequence
  Reverse primer
ggaaatgagtaccaactcg

SEQ ID NO: 692 - DNA, 20 bp, Artificial Sequence
  Reverse primer
ctcggtaacgttctctttgc

SEQ ID NO: 693 - DNA, 19 bp, Artificial Sequence
  Reverse primer
gcatgtccaacgagactgc

SEQ ID NO: 694 - DNA, 19 bp, Artificial Sequence
  Reverse primer
catctcacgacaactgtcc

SEQ ID NO: 695 - DNA, 19 bp, Artificial Sequence
  Reverse primer
cgattccagtctctgaacc

SEQ ID NO: 696 - DNA, 21 bp, Artificial Sequence
  Reverse primer
cgtgtttacagcaatctttgg

SEQ ID NO: 697 - DNA, 19 bp, Artificial Sequence
  Reverse primer
ctgcattcgtcttcattcc

SEQ ID NO: 698 - DNA, 21 bp, Artificial Sequence
  Reverse primer
gttacaggttagcttttcagg

SEQ ID NO: 699 - DNA, 20 bp, Artificial Sequence
  Reverse primer
catcagaactatgtctgagc

SEQ ID NO: 700 - DNA, 20 bp, Artificial Sequence
  Reverse primer
catcagcaacagatcaatgc

SEQ ID NO: 701 - DNA, 20 bp, Artificial Sequence
  Reverse primer
cattctgtagaatgctgagc

SEQ ID NO: 702 - DNA, 19 bp, Artificial Sequence
  Reverse primer
gaacacgtgaccgatgtgc

SEQ ID NO: 703 - DNA, 20 bp, Artificial Sequence
  Reverse primer
gtgtttggttgggaaacagg

SEQ ID NO: 704 - DNA, 20 bp, Artificial Sequence
  Reverse primer
gtatgtgtcttcaaactgcc

SEQ ID NO: 705 - DNA, 19 bp, Artificial Sequence
  Reverse primer
cctttgtcgtgatctgacg

SEQ ID NO: 706 - DNA, 20 bp, Artificial Sequence
  Reverse primer
gttgatcatccctcctgtgc

SEQ ID NO: 707 - DNA, 21 bp, Artificial Sequence
  Reverse primer
ctactggtatgatatgaatcc

SEQ ID NO: 708 - DNA, 19 bp, Artificial Sequence
  Reverse primer
ggcacatgatacattcagc

SEQ ID NO: 709 - DNA, 20 bp, Artificial Sequence
  Reverse primer
cttcttccctacaaactagc

SEQ ID NO: 710 - DNA, 20 bp, Artificial Sequence
  Reverse primer
ggtgacttctctaaacatcc

SEQ ID NO: 711 - DNA, 20 bp, Artificial Sequence
  Reverse primer
gagagcacctgtagagatcc

SEQ ID NO: 712 - DNA, 21 bp, Artificial Sequence
  Reverse primer
cttcatcaactgaaaagatgc

SEQ ID NO: 713 - DNA, 19 bp, Artificial Sequence
  Reverse primer
gtgtgcctattgcattggc

SEQ ID NO: 714 - DNA, 20 bp, Artificial Sequence
  Reverse primer
cagttctttctgtctagagg

SEQ ID NO: 715 - DNA, 19 bp, Artificial Sequence
  Reverse primer
cagtgaaacagagcagtgc

SEQ ID NO: 716 - DNA, 20 bp, Artificial Sequence
  Reverse primer
ctgactcctgaacaatgtcc

SEQ ID NO: 717 - DNA, 19 bp, Artificial Sequence
  Reverse primer
cttgagtggctttccaacc

SEQ ID NO: 718 - DNA, 22 bp, Artificial Sequence
  Reverse primer
gattgctcactggctggcttgc

SEQ ID NO: 719 - DNA, 20 bp, Artificial Sequence
  Reverse primer
ctaggagaagacatccctcg

SEQ ID NO: 720 - DNA, 22 bp, Artificial Sequence
  Reverse primer
gcaagcctgaatgtattttggg

SEQ ID NO: 721 - DNA, 20 bp, Artificial Sequence
  Reverse primer
catcttggagatatctaccc

SEQ ID NO: 722 - DNA, 20 bp, Artificial Sequence
  Reverse primer
cggaattcaacattccaagc

SEQ ID NO: 723 - DNA, 19 bp, Artificial Sequence
  Reverse primer
gtgcttagagttgcctggc

SEQ ID NO: 724 - DNA, 19 bp, Artificial Sequence
  Reverse primer
ccctaggttaacagatgcc

SEQ ID NO: 725 - DNA, 20 bp, Artificial Sequence
  Reverse primer
ccttgtccaaaacttgaacg

SEQ ID NO: 726 - DNA, 19 bp, Artificial Sequence
  Reverse primer
ctcacctggcattagatcc

SEQ ID NO: 727 - DNA, 20 bp, Artificial Sequence
  Reverse primer
cttgacaatccactgtttcc

SEQ ID NO: 728 - DNA, 19 bp, Artificial Sequence
  Reverse primer
ggtacccaggcatatctgg

SEQ ID NO: 729 - DNA, 20 bp, Artificial Sequence
  Reverse primer
gaggttgagtaacatgttcc

SEQ ID NO: 730 - DNA, 21 bp, Artificial Sequence
  Reverse primer
gaagcacagtttagaaatggc

SEQ ID NO: 731 - DNA, 20 bp, Artificial Sequence
  Reverse primer
gaaagggctcctatagatgc

SEQ ID NO: 732 - DNA, 20 bp, Artificial Sequence
  Reverse primer
cttccaccataacatttggc

SEQ ID NO: 733 - DNA, 20 bp, Artificial Sequence
  Reverse primer
cttaggatggaaaccatcgc

SEQ ID NO: 734 - DNA, 20 bp, Artificial Sequence
  Reverse primer
gaccatctaacatcacaagg

SEQ ID NO: 735 - DNA, 19 bp, Artificial Sequence
  Reverse primer
ggatggctgttgttcatcc

SEQ ID NO: 736 - DNA, 20 bp, Artificial Sequence
  Reverse primer
gtagtgcttctcagtttagc

SEQ ID NO: 737 - DNA, 20 bp, Artificial Sequence
  Reverse primer
gttgaaggaattggaagagg

SEQ ID NO: 738 - DNA, 20 bp, Artificial Sequence
  Reverse primer
cttgagcaacgactttctgg

SEQ ID NO: 739 - DNA, 21 bp, Artificial Sequence
  Reverse primer
ggaaaacttggtaaaagtgac

SEQ ID NO: 740 - DNA, 19 bp, Artificial Sequence
  Reverse primer
gtgtcaggatccctgaatc

SEQ ID NO: 741 - DNA, 19 bp, Artificial Sequence
  Reverse primer
ggactcagcactcacaatg

SEQ ID NO: 742 - DNA, 20 bp, Artificial Sequence
  Reverse primer
gattcagtgacacagaatgg

SEQ ID NO: 743 - DNA, 19 bp, Artificial Sequence
  Reverse primer
cttcctttgtgagttgtgg

SEQ ID NO: 744 - DNA, 19 bp, Artificial Sequence
  Reverse primer
ctgagacgcacagtatagc

SEQ ID NO: 745 - DNA, 19 bp, Artificial Sequence
  Reverse primer
ctgtagcttgccaatctgg

SEQ ID NO: 746 - DNA, 19 bp, Artificial Sequence
  Reverse primer
ggtctgtgcctcaatgtcc

SEQ ID NO: 747 - DNA, 19 bp, Artificial Sequence
  Reverse primer
gtcttgggtcactctgagg

SEQ ID NO: 748 - DNA, 19 bp, Artificial Sequence
  Reverse primer
gggagaacaagttctgacc

SEQ ID NO: 749 - DNA, 19 bp, Artificial Sequence
  Reverse primer
cacctctcccgaccttagc

SEQ ID NO: 750 - DNA, 19 bp, Artificial Sequence
  Reverse primer
gtggacacgtcccaaatcc

SEQ ID NO: 751 - DNA, 20 bp, Artificial Sequence
  Reverse primer
cacatgggttactcttaggg

SEQ ID NO: 752 - DNA, 19 bp, Artificial Sequence
  Reverse primer
ctctgttcctgtgcttccc

SEQ ID NO: 753 - DNA, 19 bp, Artificial Sequence
  Reverse primer
ccttccacaaactctgtgc

SEQ ID NO: 754 - DNA, 18 bp, Artificial Sequence
  Reverse primer
cagcattacagccctccc

SEQ ID NO: 755 - DNA, 20 bp, Artificial Sequence
  Reverse primer
ctctgttctctttgcagtgc

SEQ ID NO: 756 - DNA, 20 bp, Artificial Sequence
  Reverse primer
cagttctctgattgagatgg

SEQ ID NO: 757 - DNA, 19 bp, Artificial Sequence
  Reverse primer
gtacagtgaaattcagtgc

SEQ ID NO: 758 - DNA, 19 bp, Artificial Sequence
  Reverse primer
gcttgtttctacctgtagc

SEQ ID NO: 759 - DNA, 19 bp, Artificial Sequence
  Reverse primer
ctggatcctccacttgtgc

SEQ ID NO: 760 - DNA, 20 bp, Artificial Sequence
  Reverse primer
gtagtccttggaaaagtagc

SEQ ID NO: 761 - DNA, 19 bp, Artificial Sequence
  Reverse primer
gaatgggctgaatgaaggc

SEQ ID NO: 762 - DNA, 19 bp, Artificial Sequence
  Reverse primer
gatgagttctgtgccttcc

SEQ ID NO: 763 - DNA, 19 bp, Artificial Sequence
  Reverse primer
gaacactggtgaccatagc

SEQ ID NO: 764 - DNA, 20 bp, Artificial Sequence
  Reverse primer
gctgttgttctcaagttccc

SEQ ID NO: 765 - DNA, 20 bp, Artificial Sequence
  Reverse primer
cctatgaggaagacatttgg

SEQ ID NO: 766 - DNA, 19 bp, Artificial Sequence
  Reverse primer
cccttaactggcagtcagc

SEQ ID NO: 767 - DNA, 20 bp, Artificial Sequence
  Reverse primer
gagatcccattgtctttgcc

SEQ ID NO: 768 - DNA, 19 bp, Artificial Sequence
  Reverse primer
gtagtgcctacatacaccc

SEQ ID NO: 769 - DNA, 19 bp, Artificial Sequence
  Reverse primer
ctgcaagaagcattcttcc

SEQ ID NO: 770 - DNA, 19 bp, Artificial Sequence
  Reverse primer
cagtcccagcaactatgcc

SEQ ID NO: 771 - DNA, 21 bp, Artificial Sequence
  Reverse primer
ctcttggtttctttgaacagg

SEQ ID NO: 772 - DNA, 20 bp, Artificial Sequence
  Reverse primer
gattcttagtggatgttccg

SEQ ID NO: 773 - DNA, 21 bp, Artificial Sequence
  Reverse primer
ctctttcttacggttcaaagg

SEQ ID NO: 774 - DNA, 21 bp, Artificial Sequence
  Reverse primer
gatgaggaagaagacaattgg

SEQ ID NO: 775 - DNA, 18 bp, Artificial Sequence
  Reverse primer
cacttcccaggccaaggg

SEQ ID NO: 776 - DNA, 20 bp, Artificial Sequence
  Reverse primer
catccagataagcagattgc

SEQ ID NO: 777 - DNA, 19 bp, Artificial Sequence
  Reverse primer
caagctggggatgttttgg

SEQ ID NO: 778 - DNA, 20 bp, Artificial Sequence
  Reverse primer
caacactctgactttctagc

SEQ ID NO: 779 - DNA, 20 bp, Artificial Sequence
  Reverse primer
gttcatcaattgtactcagc

SEQ ID NO: 780 - DNA, 20 bp, Artificial Sequence
  Reverse primer
cacacccaatgaatgaacgc

SEQ ID NO: 781 - DNA, 19 bp, Artificial Sequence
  Reverse primer
gacatgagatccacaaccc

SEQ ID NO: 782 - DNA, 19 bp, Artificial Sequence
  Reverse primer
ctgtgtcctttgctagacg

SEQ ID NO: 783 - DNA, 20 bp, Artificial Sequence
  Reverse primer
gccctctagaagaatcttgc

SEQ ID NO: 784 - DNA, 18 bp, Artificial Sequence
  Reverse primer
gtccagaaacacccaccc

SEQ ID NO: 785 - DNA, 20 bp, Artificial Sequence
  Reverse primer
cctctacagatacatcatgc

SEQ ID NO: 786 - DNA, 20 bp, Artificial Sequence
  Reverse primer
cctgaattgaataggcaccc

SEQ ID NO: 787 - DNA, 19 bp, Artificial Sequence
  Reverse primer
cagaagtcaccagagatcc

SEQ ID NO: 788 - DNA, 19 bp, Artificial Sequence
  Reverse primer
gaccaagaaaggcagtagc

SEQ ID NO: 789 - DNA, 18 bp, Artificial Sequence
  Reverse primer
gagagccaaacacctccg

SEQ ID NO: 790 - DNA, 19 bp, Artificial Sequence
  Reverse primer
caatggcataggcttttgg

SEQ ID NO: 791 - DNA, 21 bp, Artificial Sequence
  Reverse primer
gtggactcttttctcataacc

SEQ ID NO: 792 - DNA, 21 bp, Artificial Sequence
  Reverse primer
gtttcagtgtttacatactgg

SEQ ID NO: 793 - DNA, 19 bp, Artificial Sequence
  Reverse primer
gaactctgtctctgagagg

SEQ ID NO: 794 - DNA, 19 bp, Artificial Sequence
  Reverse primer
gagcaaagtgcttgttggg

SEQ ID NO: 795 - DNA, 22 bp, Artificial Sequence
  Reverse primer
ctgttgttttgtttcaaacagg

SEQ ID NO: 796 - DNA, 21 bp, Artificial Sequence
  Reverse primer
gagttgtagtctcttaactgc

SEQ ID NO: 797 - DNA, 19 bp, Artificial Sequence
  Reverse primer
cttcggcaaccacaagtcg

SEQ ID NO: 798 - DNA, 21 bp, Artificial Sequence
  Reverse primer
caaccttccctaatgttttgg

SEQ ID NO: 799 - DNA, 18 bp, Artificial Sequence
  Reverse primer
gtcagccctgagaaagcg

SEQ ID NO: 800 - DNA, 20 bp, Artificial Sequence
  Reverse primer
gcatagaagatactctgacc

SEQ ID NO: 801 - DNA, 20 bp, Artificial Sequence
  Reverse primer
gtgtatctcacttgcatgcc

SEQ ID NO: 802 - DNA, 20 bp, Artificial Sequence
  Reverse primer
gttctcaccctaagtcatgc

SEQ ID NO: 803 - DNA, 20 bp, Artificial Sequence
  Reverse primer
ctttcagttatgcacgtgcg

SEQ ID NO: 804 - DNA, 19 bp, Artificial Sequence
  Reverse primer
catcaaaccatgcccatgc

SEQ ID NO: 805 - DNA, 20 bp, Artificial Sequence
  Reverse primer
gactgaatggtaactggacg

SEQ ID NO: 806 - DNA, 21 bp, Artificial Sequence
  Reverse primer
cattactcaaatggggtctgg

SEQ ID NO: 807 - DNA, 19 bp, Artificial Sequence
  Reverse primer
gaggttgacagagggtagg

SEQ ID NO: 808 - DNA, 18 bp, Artificial Sequence
  Reverse primer
ctccatttgggctagtgg

SEQ ID NO: 809 - DNA, 20 bp, Artificial Sequence
  Reverse primer
catctacactaagaagaagc

SEQ ID NO: 810 - DNA, 20 bp, Artificial Sequence
  Reverse primer
caacttcagctaatccatgc

SEQ ID NO: 811 - DNA, 20 bp, Artificial Sequence
  Reverse primer
gatcaatcttatgccagagg

SEQ ID NO: 812 - DNA, 21 bp, Artificial Sequence
  Reverse primer
ctaagaattgctttctgatgg

SEQ ID NO: 813 - DNA, 20 bp, Artificial Sequence
  Reverse primer
gtgaattccagtgtcaatcc

SEQ ID NO: 814 - DNA, 19 bp, Artificial Sequence
  Reverse primer
gtgaccttttctccatccc

SEQ ID NO: 815 - DNA, 19 bp, Artificial Sequence
  Reverse primer
ctgactgagtgggagaacc

SEQ ID NO: 816 - DNA, 20 bp, Artificial Sequence
  Reverse primer
ggacacttgaaactattgcc

SEQ ID NO: 817 - DNA, 20 bp, Artificial Sequence
  Reverse primer
gtgattggaaacgaaagtgg

SEQ ID NO: 818 - DNA, 20 bp, Artificial Sequence
  Reverse primer
cctgactttcctaaagatgg

SEQ ID NO: 819 - DNA, 19 bp, Artificial Sequence
  Reverse primer
ccaaaacgagcccagcagc

SEQ ID NO: 820 - DNA, 22 bp, Artificial Sequence
  Reverse primer
ctattgtttgtcttaaggaagg

SEQ ID NO: 821 - DNA, 20 bp, Artificial Sequence
  Reverse primer
gaaagtgaacaggtcacagg

SEQ ID NO: 822 - DNA, 20 bp, Artificial Sequence
  Reverse primer
cagtttactctaacatcacc

SEQ ID NO: 823 - DNA, 18 bp, Artificial Sequence
  Reverse primer
gccagaaacatccatggc

SEQ ID NO: 824 - DNA, 20 bp, Artificial Sequence
  Reverse primer
cagactaagttccttgttgc

SEQ ID NO: 825 - DNA, 21 bp, Artificial Sequence
  Reverse primer
gacttatggtggtccttaagg

SEQ ID NO: 826 - DNA, 19 bp, Artificial Sequence
  Reverse primer
gactcttaggcaacttggc

SEQ ID NO: 827 - DNA, 19 bp, Artificial Sequence
  Reverse primer
gcaatgtcctgccttttgc

SEQ ID NO: 828 - DNA, 20 bp, Artificial Sequence
  Reverse primer
ctgaaatcaaaggggttagc

SEQ ID NO: 829 - DNA, 20 bp, Artificial Sequence
  Reverse primer
cctatgtggcaagtaaagcc

SEQ ID NO: 830 - DNA, 18 bp, Artificial Sequence
  Reverse primer
ctgtgaggtgggatcagg

SEQ ID NO: 831 - DNA, 20 bp, Artificial Sequence
  Reverse primer
cggtagtgctctttcaaagc

SEQ ID NO: 832 - DNA, 19 bp, Artificial Sequence
  Reverse primer
caagggtggagttggaagg

SEQ ID NO: 833 - DNA, 20 bp, Artificial Sequence
  Reverse primer
cttccttgacctcttctagg

SEQ ID NO: 834 - DNA, 19 bp, Artificial Sequence
  Reverse primer
ccatcgtgctgttcagtgg

SEQ ID NO: 835 - DNA, 19 bp, Artificial Sequence
  Reverse primer
gtgcagactgcatctgtgg

SEQ ID NO: 836 - DNA, 20 bp, Artificial Sequence
  Reverse primer
gtttgacacagagccattcc

SEQ ID NO: 837 - DNA, 18 bp, Artificial Sequence
  Reverse primer
gcacttaccagtgacacc

SEQ ID NO: 838 - DNA, 19 bp, Artificial Sequence
  Reverse primer
cctctggtaggaaaactgg

SEQ ID NO: 839 - DNA, 19 bp, Artificial Sequence
  Reverse primer
gctaccctcatttcaaggc

SEQ ID NO: 840 - DNA, 19 bp, Artificial Sequence
  Reverse primer
ctgtccactagtcaacagg

SEQ ID NO: 841 - DNA, 18 bp, Artificial Sequence
  Reverse primer
ggcaattgtggacactcg

SEQ ID NO: 842 - DNA, 20 bp, Artificial Sequence
  Reverse primer
cacagcttatccccaaaagc

SEQ ID NO: 843 - DNA, 18 bp, Artificial Sequence
  Reverse primer
ccaaccactctgggttcc

SEQ ID NO: 844 - DNA, 19 bp, Artificial Sequence
  Reverse primer
caagagtccaaggactagg

SEQ ID NO: 845 - DNA, 21 bp, Artificial Sequence
  Reverse primer
gaagagaaagtgaatcttccg

SEQ ID NO: 846 - DNA, 19 bp, Artificial Sequence
  Reverse primer
gaagactctggtgttgtgc

SEQ ID NO: 847 - DNA, 19 bp, Artificial Sequence
  Reverse primer
cagaagtctccaaaagtgg

SEQ ID NO: 848 - DNA, 20 bp, Artificial Sequence
  Reverse primer
gaaaccaaatgtatccaggc

SEQ ID NO: 849 - DNA, 20 bp, Artificial Sequence
  Reverse primer
ctttgttcatgtctgactgg

SEQ ID NO: 850 - DNA, 19 bp, Artificial Sequence
  Reverse primer
ggttgtgagctgtcagagc

SEQ ID NO: 851 - DNA, 20 bp, Artificial Sequence
  Reverse primer
gattcactttagacctcagc

SEQ ID NO: 852 - DNA, 18 bp, Artificial Sequence
  Reverse primer
ggaggccttgtactaggc

SEQ ID NO: 853 - DNA, 21 bp, Artificial Sequence
  Reverse primer
caacattagagactattccac

SEQ ID NO: 854 - DNA, 21 bp, Artificial Sequence
  Reverse primer
ggtatatccagtaatgaaagg

SEQ ID NO: 855 - DNA, 20 bp, Artificial Sequence
  Reverse primer
ctcaagaaagatgcaagacc

SEQ ID NO: 856 - DNA, 22 bp, Artificial Sequence
  Reverse primer
gaactattctacacatttcttc

SEQ ID NO: 857 - DNA, 20 bp, Artificial Sequence
  Reverse primer
gacttaaacctccctaatgc

SEQ ID NO: 858 - DNA, 21 bp, Artificial Sequence
  Reverse primer
ctgaagcatgatatacaacac

SEQ ID NO: 859 - DNA, 20 bp, Artificial Sequence
  Reverse primer
ggctattgttacctcagttg

SEQ ID NO: 860 - DNA, 20 bp, Artificial Sequence
  Reverse primer
ctctgtaccacatgagtatc

SEQ ID NO: 861 - DNA, 22 bp, Artificial Sequence
  Reverse primer
gtgtttatcttctaaaagtgac

SEQ ID NO: 862 - DNA, 20 bp, Artificial Sequence
  Reverse primer
ggtcataatgcccaaacttg

SEQ ID NO: 863 - DNA, 21 bp, Artificial Sequence
  Reverse primer
ccattctttcactaacatgag

SEQ ID NO: 864 - DNA, 20 bp, Artificial Sequence
  Reverse primer
gttctcatgaaatccttggg

SEQ ID NO: 865 - DNA, 106 bp, Artificial Sequence
  PCR amplified product
catgaagttatggggttaggtgctagtttcctcttgaaggagaaacagatagtttgagtgtgtcagcatgttagatgatgaccatatcgtatgtgcctgccacatg

SEQ ID NO: 866 - DNA, 109 bp, Artificial Sequence
  PCR amplified product
gaaccatctctttctttccctgtttcatgctctgcatacacttttctcgcccagcttagagtgttagcttggagcatccttgtttcaagaccatgagctctgaggtcag

SEQ ID NO: 867 - DNA, 108 bp, Artificial Sequence
  PCR amplified product
gccaacagagacctgacctggtgtggaataggtatgttggatatgcttgtgaatgcctggccaggcaggatgtgttttgaggctcactgcaaatggatgcaagttctc

SEQ ID NO: 868 - DNA, 114 bp, Artificial Sequence
  PCR amplified product
cctgtttacagccctttccaattcacaggccaaacaggaaaggggggaggggttagagaagggcacaaatgtcagaaatcacaaatcatacagttgctcctcaaagcaataggc

SEQ ID NO: 869 - DNA, 119 bp, Artificial Sequence
  PCR amplified product
gagcttctgttgagtgacccattgatagactggcttcttctctttgccccaactagacccctctgtgagctgtttgtgctgaccttgggctgggaagatgctctcaaacacctgttgtg

SEQ ID NO: 870 - DNA, 102 bp, Artificial Sequence
  PCR amplified product
cttggaggcagcatgtggggagaggtcaacctctaaagtgccagctctccagaaatgcagccggaatgaaggtttgaagggatggtagtgaatggagcactc

SEQ ID NO: 871 - DNA, 113 bp, Artificial Sequence
  PCR amplified product
ctaccctctagtgatgagggtcccttggcctggaaaaggggaaggaggagatagggggctaggccttgaaggaagtcaaaccctaagacaagaggatcagatttggggtgctg

SEQ ID NO: 872 - DNA, 142 bp, Artificial Sequence
  PCR amplified product
caccctccttggtaagcccccatcctaacccttttgtgtggtaaagacacatgtaaactgtcccaaaacaaaagacagagagcagagactaccagagggtgagtggaggtacttgggtgggtctggctagtggggaaacacc

SEQ ID NO: 873 - DNA, 116 bp, Artificial Sequence
  PCR amplified product
gttgggctggtgcttggcagggtgtgcaaagggtttcaggccagattagtggaggttgagtggggattggagggtaggggtggattgtcatgtgagcctcacaagaatacaagaag

SEQ ID NO: 874 - DNA, 123 bp, Artificial Sequence
  PCR amplified product
cagatgaggaaaccaaagggcagaaacatttttaggagaacatacaagagggaatgggaatttgtattctccaagtccagggcctcactctgctgcaccctgcacgtttcaggacagcctttc

SEQ ID NO: 875 - DNA, 110 bp, Artificial Sequence
  PCR amplified product
gacaccatcacgttttcagctgacactcaataccagaatcatcgcttgccccttgtatttgtggccagtttattttaaaaatgcttctgtgctttggtttcctcaactgg

SEQ ID NO: 876 - DNA, 107 bp, Artificial Sequence
  PCR amplified product
ggtgacatggtactagggatcagatgccagaagaatgggggcaagaccttgtgaaataggagttggggttaaggtcagccttgtgttggcagtgtctcctctagcag

SEQ ID NO: 877 - DNA, 111 bp, Artificial Sequence
  PCR amplified product
catgtgagtggctatacaagccaacctaggctcaaaatgtgcagtgatagggactattgcctgtgatcacaaattttcgcatcttttattttcttcagttccagcaggttc

SEQ ID NO: 878 - DNA, 110 bp, Artificial Sequence
  PCR amplified product
gtcactctgaatatctgaggttcagtgcattaacgtggagcacagtgtttgttttgaaagtcattcatgaaataataaccaatgttcaccagcacaataagttctatgag

SEQ ID NO: 879 - DNA, 116 bp, Artificial Sequence
  PCR amplified product
ggtagaatagaaagaaacaccaggatatgacatttagaaatggcctatcttcagatgtaaagaactatttgggttaattttttaattgataatttgatgagatttagggatattgg

SEQ ID NO: 880 - DNA, 119 bp, Artificial Sequence
  PCR amplified product
ggtatgtgaatctatttgcacaaagtagcatgctgagtattgtacaaagcacactaaacatcttttaagtcactttgaaaatggcaacagtctcagggaggaacatacagaaggctatg

SEQ ID NO: 881 - DNA, 101 bp, Artificial Sequence
  PCR amplified product
gaacatacagaaggctatgctgctatgttgatacatctaagacaactgagagaaaaaaatatgcagggtaaaataaacttcccattacaaaagcaacaaag

SEQ ID NO: 882 - DNA, 88 bp, Artificial Sequence
  PCR amplified product
cactcaaccattcagtcttccaagttagaatgaatgactaagccatgattcgtttttcttgctttgatctatcagccactaaatcctg

SEQ ID NO: 883 - DNA, 99 bp, Artificial Sequence
  PCR amplified product
cttgtaagttccaacatcttcagagcatcagtcagttttagcagtgtcattctgaaggcactccaaagccaccgctgactgcatcttatagaaattccc

SEQ ID NO: 884 - DNA, 98 bp, Artificial Sequence
  PCR amplified product
cctcaacagcatgaattagccccagattctttacctgctaccaaagctagcccagaggaagaggaacagagagcggacaagctttgataggaattgtg

SEQ ID NO: 885 - DNA, 118 bp, Artificial Sequence
  PCR amplified product
gtagaccaaaggaagaatggaatctgcatcagctattctttcctgaacacagaccctagaatatattttttctagaagtttttatatcatagtatcaagagtcatggttttctgactc

SEQ ID NO: 886 - DNA, 114 bp, Artificial Sequence
  PCR amplified product
cagctgatcaagtgaagcggcctttccacagactattgacgatctgtctcaagcattatctcataagtttccttttattttctctcccaacccagatagtcttcgctttgaacc

SEQ ID NO: 887 - DNA, 114 bp, Artificial Sequence
  PCR amplified product
gatcttcatggacacaagtcttgtctgtgaagataaaggaaagtaaaatcacttatgcaaaagtagatatttgtgacagactcctggatggacctcaacaaataggtctaatcc

SEQ ID NO: 888 - DNA, 103 bp, Artificial Sequence
  PCR amplified product
gatgagtgcagatttgaaggggagatacaagatgtgaacattggaagcaaccaccataggattcattacatcaatcatggatgctttacttgtaattgttccg

SEQ ID NO: 889 - DNA, 105 bp, Artificial Sequence
  PCR amplified product
cagctttgctttgcttgggacatgtgattctgacccagtaccccagacctgaaggcccctctatgtgtcagtcctgaaaggattcgctgttttcttagcctcatg

SEQ ID NO: 890 - DNA, 112 bp, Artificial Sequence
  PCR amplified product
gtgctgcattagagtttggtcacaggcttgactaggtggaagagctttctgagagttgtgtgcaaaaaaacacttagctgccgttccatatttgcttgcttgatccccttgg

SEQ ID NO: 891 - DNA, 121 bp, Artificial Sequence
  PCR amplified product
gtatgatagagttttccttcctgaggttgaccatggcttagtccttgctatacagggtagtgtgaggattggatttctcaggttagcggagctttagacgagcgttgagactgtctgtctc

SEQ ID NO: 892 - DNA, 139 bp, Artificial Sequence
  PCR amplified product
cagatgtgttttgatttcagccaagaacaaagatatttgatatgtcaattgcccagtgtttaggaaaaaggataattttggttactgcttttgaactagtggtgggaaccttggaaatcccccacagtctggggacccc

SEQ ID NO: 893 - DNA, 120 bp, Artificial Sequence
  PCR amplified product
gcaagacttcctcgtttggattttggttttcagattggttgggggaaatctcacatacgggaagaagaagaaaaacaaatataagtaagtttccctttggggccatggtccacttacagc

SEQ ID NO: 894 - DNA, 115 bp, Artificial Sequence
  PCR amplified product
ctgcagtttgccaaagtcgcattggcaggatagacttggaaatgcaagggtgcttggcatctcccatcaagttggcatttccctggctttagctttcgacaggaaccagctgtag

SEQ ID NO: 895 - DNA, 121 bp, Artificial Sequence
  PCR amplified product
cttgcaggccatggaaggggaccctacatctttcctgttagcactgcgggtggctttgtttaagcaatgagctatgagagaacatctcccctcctgctgtgtgcgctctactgctacagtg

SEQ ID NO: 896 - DNA, 110 bp, Artificial Sequence
  PCR amplified product
ctggagctcctgaattgggagggtcttcatctttctccggcttcaaccttaagtctgctctccaaatgacttgataacaccataggaaccaaagattgtaaagcaaagtc

SEQ ID NO: 897 - DNA, 117 bp, Artificial Sequence
  PCR amplified product
gatgcacctgtgctattgcctcctctgttaggaggcatggataccccccagcctcctggaaagctgaacatagggagttaaagggttgttctccaccgggagtcaatcgacccaatc

SEQ ID NO: 898 - DNA, 101 bp, Artificial Sequence
  PCR amplified product
gcaaacacctacacgttggtacatatccccagttccttcccaggcactggccttatgcccagcacccggaaactctttggaaggtaggcgttcatgtgaag

SEQ ID NO: 899 - DNA, 118 bp, Artificial Sequence
  PCR amplified product
ggtattgttgtcatccaagccagaggaataaaccatgcataagatagtcaaaagcactgcatatcaggtgggaggtgggagggtagggactccaacctgggacaaagtaaagtaaagc

SEQ ID NO: 900 - DNA, 115 bp, Artificial Sequence
  PCR amplified product
ctccagatgcctcaacaggcatagctcaggccaagaaagacggctcctcaaatgtccagcatctgcccatcatgcatcaccccttacatgcagagcccatccactccacagttac

SEQ ID NO: 901 - DNA, 109 bp, Artificial Sequence
  PCR amplified product
cacaaaactaaagttgactccaaatgtctttgccaactcaacaggataatattaaatgcggaatattttgttccccttgtacctctccaggtcagaagcataggaggag

SEQ ID NO: 902 - DNA, 114 bp, Artificial Sequence
  PCR amplified product
gttaccaccttccctcttgccatttttaatttatgagttcaatactttcttctccggtctcttcctttcctaagagatttcaagtcaatttccatggatcatagtccacctgag

SEQ ID NO: 903 - DNA, 109 bp, Artificial Sequence
  PCR amplified product
gagttttcaacctggctagcctaggacacaggaaggtgtgctctaagccagaaggagaatagacttcctagttttaatgcactccatttggatttctctacccattgag

SEQ ID NO: 904 - DNA, 109 bp, Artificial Sequence
  PCR amplified product
ctgagtatgcaaacagcaccatttgatacatgcaaagcaagaacccatgctgcttaaaccagttattctcgttcacccataggggcattcccaactcatggccaagtcc

SEQ ID NO: 905 - DNA, 110 bp, Artificial Sequence
  PCR amplified product
cagttccacctttccaggctcttacatacttgggattggcccacagggacactggattaaaggttccacttgaaaaataaggtcccactgggctcaagagtacaaagagc

SEQ ID NO: 906 - DNA, 116 bp, Artificial Sequence
  PCR amplified product
catccttctgtttcatagcctaagtgactgcttcaggacatggcagggtcttcagcaggtggtaggtgcaggcgaatgtgtcattagcacacctgcccacatcaactagcaggtag

SEQ ID NO: 907 - DNA, 109 bp, Artificial Sequence
  PCR amplified product
gtttccttcattccatgttccaagtaatgccattcttagtaacctatacccaggtttctgtctctgttccatgggctgctgggttgggggccatgggaattgggagaag

SEQ ID NO: 908 - DNA, 125 bp, Artificial Sequence
  PCR amplified product
caaagtgactgtgtccaagcccgtgtgggacttcctgagcaaagagaccccagcccggctggcccggcttcgggaggagcaccgtgtgtccatcctcatagatggcgagacttctgacatctatg

SEQ ID NO: 909 - DNA, 108 bp, Artificial Sequence
  PCR amplified product
cccagctatgagaagtacggcaccattgtcatccagtacgtcttcccgcccggtgtccagggggtaagaagaccatggcctgcccttaccctttggctttccccaagc

SEQ ID NO: 910 - DNA, 120 bp, Artificial Sequence
  PCR amplified product
gagtagtcaaggcctataggtgtcttcctgctggacaaagagtaatgtgcaattctggctgcagaggggtgaagaagctgcacagaagagtgatggaaaatggaacaagttagacagtgg

SEQ ID NO: 911 - DNA, 124 bp, Artificial Sequence
  PCR amplified product
gttcaaacagctaacaaccctcaccctcatttctctttgctcccaggtttgggtacccagaccccacctacctgacccgggtgcaagaggagctgagagcgaagggtatcacagatgactgaag

SEQ ID NO: 912 - DNA, 121 bp, Artificial Sequence
  PCR amplified product
cactcccttctggcagaggccgacctccaaggctctgttctcccctccccgtgtacatatactcccggtttccctgcccctccattgcccttggctttttctggtatgtgctgtgctccac

SEQ ID NO: 913 - DNA, 113 bp, Artificial Sequence
  PCR amplified product
caatgtctcctaacagttggcagacatgtctgaagtaaagctcaacgatgaagttctggaatctcagggccccatccagatgccccagaccacacccaccctgacaaatcaag

SEQ ID NO: 914 - DNA, 115 bp, Artificial Sequence
  PCR amplified product
ggatgacatcattccgaaggacaggcagaacccaaggtcagcaatttccgaagctcatcaccaccaactcacaccagcaggctgagaacctgccgagggttgcagattcattccc

SEQ ID NO: 915 - DNA, 118 bp, Artificial Sequence
  PCR amplified product
cactcccttggctatccgggtgtccattctaccccacgaaggtctaagggcttacagagctgcaagggaacagagagagaatgggtgatgacaggggagcgtctcatagcccaggatg

SEQ ID NO: 916 - DNA, 122 bp, Artificial Sequence
  PCR amplified product
gctgtcaaacttcaacttgctttatgagcccagaggatcctgaaacagaagcgcccacaccagtgagtcctagaggagcagtgagtcctagttgccccccgaccaatgctcattccattcac

SEQ ID NO: 917 - DNA, 115 bp, Artificial Sequence
  PCR amplified product
gcaagggtcaaacttcaacctgctactggaatgctggcacgctggttgtgaccttgctcctgaagtagctggccaacggaggtgctgccactgagcggtgatggtcaaggtcagc

SEQ ID NO: 918 - DNA, 110 bp, Artificial Sequence
  PCR amplified product
ctcctccagcagcaaaaggaaacaccagagtcctccatggctcttgcaatggagagttctttgtgtacacctcccacccgatccccttacaccagaatcccaaacccttc

SEQ ID NO: 919 - DNA, 122 bp, Artificial Sequence
  PCR amplified product
ccatactttagataggttacctatattgttactgccacaaaagagaccatagggctcatagcaacagaggcagaataaacgcctcagtgagattccaagagcattagtcatttgacagcttc

SEQ ID NO: 920 - DNA, 116 bp, Artificial Sequence
  PCR amplified product
ccagagactaagtcagaagcattttagtttaaatactgtagcatagatcatgggacataacccaggcatggaatatatatacttcaaaactatccctgctgtttctctgcattcgc

SEQ ID NO: 921 - DNA, 117 bp, Artificial Sequence
  PCR amplified product
gtgcaaagcaagcatcagggttgcctttgttggctttccaattcgttgccagcagaagcccatgtgataagaactttttgattaagctctaaatctttggcacatatctgcaatgcc

SEQ ID NO: 922 - DNA, 108 bp, Artificial Sequence
  PCR amplified product
gtatcaaaggcagtggaagctgggcaacgctcccttcctatgatcggtgtgtgagccctgacttaatgagctcctactagaggtgctaccttgaacaaacagtgcttg

SEQ ID NO: 923 - DNA, 108 bp, Artificial Sequence
  PCR amplified product
cattctgcaactgcttttcctagctcaccacaaacctcatggttgtatctctttgtcttttggactcggattcttcaagcactcgaatcctatgatcctgttgtttcc

SEQ ID NO: 924 - DNA, 112 bp, Artificial Sequence
  PCR amplified product
gtgtttgtagggtcccacgtaagcagagcagaaatcactaccgctgatcaaggagagatgaacagcatcactaaacagtgttcagagacttagcaaatcattagcaatctcc

SEQ ID NO: 925 - DNA, 128 bp, Artificial Sequence
  PCR amplified product
ggaacatctctgcatacaggtgttaaaagaagcttccacctcatgactagcataaaacttaaaccaatggttgttattcagctgaagacagtatcagtgtaaagtgccaccgatctggtcactgtctc

SEQ ID NO: 926 - DNA, 107 bp, Artificial Sequence
  PCR amplified product
cattccctaaccccacagctcaaagctttcctgtacacctgctctactcagctcatcaattttctgtgagccagttaagttcctttaagcctatccactgagaggtg

SEQ ID NO: 927 - DNA, 117 bp, Artificial Sequence
  PCR amplified product
gtagaagcttcttttcttagccaaagaaacaacacagcctgcatctccagtgtaatgccttgaccaaacatggaaatagcaatgatagggaatcagtgcctaccatgacttgaggtc

SEQ ID NO: 928 - DNA, 114 bp, Artificial Sequence
  PCR amplified product
ctagaagagaaactacaagctgcttaatctcaaagctggggtaacgtaagtaaagtgcattcaggtcgaagcctggaggagagatgacctgaagcttggtaagttcttcaaacc

SEQ ID NO: 929 - DNA, 120 bp, Artificial Sequence
  PCR amplified product
gggtacaatgaactgtaatggtgagatactcctgagatggcagccttcagaaaagactttttgacacataaagcttgtcgatactgacccttgtttgtaagcgttctttagtaatgtctg

SEQ ID NO: 930 - DNA, 104 bp, Artificial Sequence
  PCR amplified product
caaggatgcaacactgaggtggggctcccaacaggcattaccccagcaaatgaggccaaagaccacagctaaagtgatcttaaccatgttgaagaattctgaag

SEQ ID NO: 931 - DNA, 101 bp, Artificial Sequence
  PCR amplified product
ctaccctttctccaactgcccttgcttctttcacttagccataactctggcatccttcccaatttcattcacatttcgtcttggatcacacttctccaacc

SEQ ID NO: 932 - DNA, 107 bp, Artificial Sequence
  PCR amplified product
gagctgctagagcttttgcctttagccagcaatgagtagctgacgtgctctgagaattctcataggacctgacttcctggggaagttccaagtctccaaagtggttc

SEQ ID NO: 933 - DNA, 108 bp, Artificial Sequence
  PCR amplified product
gaacctgaacgtgttgaggacataaatccaacttgcttttcacttaaggatggtgagacaacctccagagacttttcctgagaatggggcaattgtcaccaactctag

SEQ ID NO: 934 - DNA, 121 bp, Artificial Sequence
  PCR amplified product
cagatcatagattgtggaggagtatgtttgatctacctaatgtttgaagctgatagaagatgaaaggggggagggagcctcaggctgtttaccaagtttcatcgtgaggtgacatttgctc

SEQ ID NO: 935 - DNA, 121 bp, Artificial Sequence
  PCR amplified product
ctggagtagagtctgggctgagggtgtagttgatttcactgggtcttgaggatctggggctctgtactgttgccaacttgagcagtaggtaaagtcctaaaggttcacttatcctcacttc

SEQ ID NO: 936 - DNA, 116 bp, Artificial Sequence
  PCR amplified product
gggtgggacctagaaagcatgttgagttgaggagtcgagagggcaggttcaaattaccacgtatatgtaatattaccatgtgttattctcatgacccttttgtcttccttgtcaac

SEQ ID NO: 937 - DNA, 108 bp, Artificial Sequence
  PCR amplified product
ctaactgtcacctccttggactgaggcttagagtttagggttttcaggatagagagcttatctgttaggtcctttgaaccgctccctagcattctggcaatagaggag

SEQ ID NO: 938 - DNA, 117 bp, Artificial Sequence
  PCR amplified product
catagaaatcctaacatcttcccctccctcccaagccttttcagttccctacactttccccccaaccctgttcccagggtatagcggcaatagagcagctaagtctgtttggtattc

SEQ ID NO: 939 - DNA, 121 bp, Artificial Sequence
  PCR amplified product
caaggccttgatgtagtgcctgcatagctagcaaagaatacgtgagcagctagtcattcctatcctagggaagctcctgagcccatgagcatggggaaaatcccagaggccttcaggaaac

SEQ ID NO: 940 - DNA, 108 bp, Artificial Sequence
  PCR amplified product
gtaacccgtctaagatgtggtgcaggatatgttcaagtctcaacccaggcaagagctctgtgatgagaagttgactattaatggctgggtggccttcacctggcatgc

SEQ ID NO: 941 - DNA, 113 bp, Artificial Sequence
  PCR amplified product
ccctgctttgagtaactcccaacacagtctccgtgccctcaaggctatgtgtatttctcactttccctggaactagtcactcatggacactctgcactcctacttctaaactg

SEQ ID NO: 942 - DNA, 115 bp, Artificial Sequence
  PCR amplified product
ctatccttcagttttctaaccttctgtctcttttggtcctaccttcagctcaagggcttaagaaagaagatatttcttttggggaagatgatttaacctttgattggacctccag

SEQ ID NO: 943 - DNA, 106 bp, Artificial Sequence
  PCR amplified product
gataggacccagtgtattgcaaggcattacatgacggactggacccaattcaagctctggtacttgttcccgaggccagaagacaagcctttcttgcaaactgcac

SEQ ID NO: 944 - DNA, 108 bp, Artificial Sequence
  PCR amplified product
ctaagacgaagtcctcagctctccaatactgcagagatggtgtctcattctgagatatcctgcagcacaccagaggcctcaacagtgttcccaaagaaaggacagccg

SEQ ID NO: 945 - DNA, 105 bp, Artificial Sequence
  PCR amplified product
gtgcactgtcaatacaacgtcccggatgcacccagctaacctcatttgggaaggcaaaattaattagtttgtgttttaacacccagctgttatcctaaggaaagg

SEQ ID NO: 946 - DNA, 114 bp, Artificial Sequence
  PCR amplified product
gagctctggattcattccggagcctcatttgctgttaacaccttttccagttagcaattctgggtgaaaagcctggccccagatctgagaggttgggcatttctattcctgtcc

SEQ ID NO: 947 - DNA, 108 bp, Artificial Sequence
  PCR amplified product
gcagtcatagttcttgaggccctcagcacagaggcagcaggaccaacgaccttcccaggagcccacagatcagcgggaaaggcaggtgtgcacaatgcataagactgg

SEQ ID NO: 948 - DNA, 102 bp, Artificial Sequence
  PCR amplified product
gctgatggtaatcatctggaggtggttaacagtctgactggggagatgacagtagaacaaaggcaatatttccaggaagacaggatgcacgtgcttagtttc

SEQ ID NO: 949 - DNA, 118 bp, Artificial Sequence
  PCR amplified product
catctgtcagcaaactgttccaggcagacccaattcttagcaccacaataaaatgaaggacatcaggataatccatcaaacaaaagcagctgggagcaccatcgtctaccaaagactg

SEQ ID NO: 950 - DNA, 117 bp, Artificial Sequence
  PCR amplified product
ctatgggtatgatatgttcggccctacagcaatactttgtcctcctacattatttaagcagagctcattaagggactggacaaccagatgacagcccagggtgccacctgtggctac

SEQ ID NO: 951 - DNA, 113 bp, Artificial Sequence
  PCR amplified product
gtagggaacatgcaaatccctcttctgttctgttctacattcaccccccagagcattctggatgcttctcagaatttccaaatcctattcatcccctccctacagaatggaag

SEQ ID NO: 952 - DNA, 120 bp, Artificial Sequence
  PCR amplified product
caaccactatgtcacaaagcccaagaacagagcccatggctgactgaagtcagcagttgcaatcaggataattctgtaactgaataatgcatgctggaatgctgtttacaggaaatgagc

SEQ ID NO: 953 - DNA, 115 bp, Artificial Sequence
  PCR amplified product
gtcttcatccatcagactggaccagctccccatgaaggctgagaaaatagtcaagtaagaaaataggagggtagccaagaccggctgccctctctaggaaagatgcatagtctcc

SEQ ID NO: 954 - DNA, 108 bp, Artificial Sequence
  PCR amplified product
cactcaatagactttcagggaaatgcaatagctcaggcaaaccttgcttacctcaaacttttactaagcaaataaacagattttgaaagtcggctgcccactgaccag

SEQ ID NO: 955 - DNA, 116 bp, Artificial Sequence
  PCR amplified product
gaggctataggttaagaggagataacagacatgctcagaaacaaatcatttcctgatagctgtttcagatggaaccaaatggaaaacagtgcttctttccgtttactgtacacccc

SEQ ID NO: 956 - DNA, 112 bp, Artificial Sequence
  PCR amplified product
gtctccactggaagaagagcctgtagaatatgcagactattcaaatgcttcctttggtccattgttgtcctttcttttctcttctagaactttccccatagtcatgcatctg

SEQ ID NO: 957 - DNA, 115 bp, Artificial Sequence
  PCR amplified product
cctgttttcccaagtttacctgcctctctgagaagcccatcagccctgagagatacctggaaggaaagaggaaaatgcgtgattcaaatcatgttgcaagtggatgaatctagcc

SEQ ID NO: 958 - DNA, 109 bp, Artificial Sequence
  PCR amplified product
ggtgttaggttcccacaggatgaaggatcaccatgaacggtcaggacctgacttaggaggactcagaagctggagactgcagaggatggcacagagttccaaatctttg

SEQ ID NO: 959 - DNA, 123 bp, Artificial Sequence
  PCR amplified product
caagtgaatgagtgaatgggcgatttccagactatccagaaactgtgccccatagtcctacccgtaggaatccaacagggaactgtcaccaccgacccgaggcaggaaccatgtgctctgagc

SEQ ID NO: 960 - DNA, 108 bp, Artificial Sequence
  PCR amplified product
gagcagtcaggggactccctggctgtttctgtgtctggtcttatgggtctgggcactgaattcagtcacaaaccctagcatgctcctttgctacctcaggggacaaag

SEQ ID NO: 961 - DNA, 110 bp, Artificial Sequence
  PCR amplified product
ccagaccaagtgacagtggtgactgccaggaacgttagccccctgaagtatcagcgtttgagttctctgggcattctgtgggccctgcagtggcacttgtccaaaagtgg

SEQ ID NO: 962 - DNA, 110 bp, Artificial Sequence
  PCR amplified product
ctggccatgagtactttcctcttgtcttgtcccttaagggttactttttgcagtgcagcaagagagaccgacatcaaccctgagttacaagcaatagatagtgatggctg

SEQ ID NO: 963 - DNA, 124 bp, Artificial Sequence
  PCR amplified product
ctcagcttttggaatgaagctatgtcaaaaagacctgcctgggtgaacccctgcaaatggaggtcagctggacctcagtaaaagcccagtgggaaggagcggggacgattctggcgatcacatg

SEQ ID NO: 964 - DNA, 121 bp, Artificial Sequence
  PCR amplified product
caccatgtactcttcacaggcaggtgtcttctggtggacccaactctgttggtacttgtcgtctccaaaaagtccccaaatgcgctagaggccagccagcccggtatccgagaggtaagag

SEQ ID NO: 965 - DNA, 113 bp, Artificial Sequence
  PCR amplified product
gtggagttgatcatttgaggcctttggctaagagggacggtggttatgtgctgggagtgggcagaggtctgggaggctttctggcagattatccagagaatcatcaagaaagg

SEQ ID NO: 966 - DNA, 117 bp, Artificial Sequence
  PCR amplified product
cactagtatgtagagtgtgggaaagccttgagatggaattctcaccttcgagttcatcaggaaattcacactggagaggacctttgaatgccagtgtgggaaagaccagcccagttc

SEQ ID NO: 967 - DNA, 108 bp, Artificial Sequence
  PCR amplified product
gtgttttgaagctaagatgcgttcagccctcccagaatcttagggattatatgaatcctctatttaaattctgttcccagccctgagggttgatcacagaaacagtag

SEQ ID NO: 968 - DNA, 115 bp, Artificial Sequence
  PCR amplified product
gactaatgtaaaccacctggttggtaagagaatgagaattctcctgtcctggagaatcagttcttgggtgtttggatcatcttactggtggtcttgcttagaaatgggatactcg

SEQ ID NO: 969 - DNA, 113 bp, Artificial Sequence
  PCR amplified product
cgtactatgtctgttcacccaccccaacaccaacagcgtaggaggagatgacttatgccctccagtgctacttataaatggtagttttcccttccttctttcaaggactttcc

SEQ ID NO: 970 - DNA, 107 bp, Artificial Sequence
  PCR amplified product
ggatgggaatggagtgacgagtccctggggaggagtacaggtgcttatctgaaagtcagaactcttgaattctagacctgcttctgaccttagaaaagcagattacc

SEQ ID NO: 971 - DNA, 105 bp, Artificial Sequence
  PCR amplified product
gagctttcatttcacatgggcccgaagttgctttctctaggatcagccacccagacttgaatcttccatccccttgtctcctttccccacaggacccttttggag

SEQ ID NO: 972 - DNA, 120 bp, Artificial Sequence
  PCR amplified product
ccattcatcccgtatcaggggccaaggtacctttacaggagcacctagagcgagggcctttggcaaaaacaaaacaaccaacacacctctccacagggccagctccttagggataagtgg

SEQ ID NO: 973 - DNA, 137 bp, Artificial Sequence
  PCR amplified product
ctgggaataggatccttaggaataaatatttatgttcacggacattagtctaaagtggcatctttaaacctaccttttttgtgtgtgatagaaacatagagttacaccttatggtgaccgcctcatccatttcacac

SEQ ID NO: 974 - DNA, 110 bp, Artificial Sequence
  PCR amplified product
caagtctctgctgagaagggctggcacatttctttcctgtgtcctctgttaggggatagcgatagactcctcgtaaactccaggatggagcctagttaggcctgtaagtg

SEQ ID NO: 975 - DNA, 132 bp, Artificial Sequence
  PCR amplified product
gtgaagtgattccaagaatccagtagttaagtctgtatggctcccaaaacccatgtccccttctctgcctaatcttccttaataaaaagccagttgatagtttttctttgctgagctctagcagccagttgg

SEQ ID NO: 976 - DNA, 111 bp, Artificial Sequence
  PCR amplified product
gaagcaaatgttcagaagggaatgaaggtcctgccatcaggacaagacatttgggtagtagagcacataattccttaccaggtatgatttgacctgaatgcatgctagctc

SEQ ID NO: 977 - DNA, 112 bp, Artificial Sequence
  PCR amplified product
cagaggtggagtaaagtggatttcacagaacatttctccgtgattgcaattctcaggctgagatggacaagaaatgctgatacatctctgcccactgatttgacaacttgag

SEQ ID NO: 978 - DNA, 119 bp, Artificial Sequence
  PCR amplified product
ctgctctcctagtgttgcctcttggccttctgtctgtgaccattctacttaaagaaacttagggaagaaggaagataaatattcgctttccttttcttggccatagaagcaccaagttc

SEQ ID NO: 979 - DNA, 113 bp, Artificial Sequence
  PCR amplified product
gtcgatgagtgaggtttccctcacacatgccatcttcccctacctctcctcttgaaaacaatgtcttttgcaccctcaaggtcaaggttaaacccgagttggtactcatttcc

SEQ ID NO: 980 - DNA, 127 bp, Artificial Sequence
  PCR amplified product
caaggaaagctctgaattgcgctcgctgtttggtttttgcttgttgtagttactctgggggaagagccgggggcaagggggtcaaatggggctaaagtttcagatttgcaaagagaacgttaccgag

SEQ ID NO: 981 - DNA, 117 bp, Artificial Sequence
  PCR amplified product
gaaccctgaaggcatagccatcttgagagttgacccagcgtttccctttcatttatttatataacctgggaaaatcttttccctttagtgtcacccttgcagtctcgttggacatgc

SEQ ID NO: 982 - DNA, 109 bp, Artificial Sequence
  PCR amplified product
ctctggccattgactttggcgtgacctttcttttcagtgcttctgattttcgctctctgcagatactcaagtaactgtgcctttctaacaggacagttgtcgtgagatg

SEQ ID NO: 983 - DNA, 108 bp, Artificial Sequence
  PCR amplified product
ccagttctcaccggaaaggcgttgtgactgtagtaagtgctgagggttgagaggagagattgagagttgttaggggaactgttacacagggttcagagactggaatcg

SEQ ID NO: 984 - DNA, 108 bp, Artificial Sequence
  PCR amplified product
gcatccagggctgaaaccaaggcagctgatgccgagaggagccaaagggcagttcttcttagtttagaaacagcaagacagcctctgccaaagattgctgtaaacacg

SEQ ID NO: 985 - DNA, 113 bp, Artificial Sequence
  PCR amplified product
gccaatgcatttccaagcccggatccaaccgtggaccctggccttttgggccagcagaagaggtggctgttttttctcatgaaatatttttgaaggaatgaagacgaatgcag

SEQ ID NO: 986 - DNA, 116 bp, Artificial Sequence
  PCR amplified product
cttgcccatggaatgaagccccccatccaatcccctccccagggaacagctttatactaactctggtggtcggcttttggaggggccataaatggcctgaaaagctaacctgtaac

SEQ ID NO: 987 - DNA, 106 bp, Artificial Sequence
  PCR amplified product
gatccaggtgtatctctgcaagtagagcttgtagcatacataaggccctgcaaagggatttctgggccggaagtttttgatcagttgctcagacatagttctgatg

SEQ ID NO: 988 - DNA, 112 bp, Artificial Sequence
  PCR amplified product
gacgacatcggaggatccgactcaggttacggcaggagagggaggccaggccgggttagggttctggggtttgggattctcttccgaggctggcattgatctgttgctgatg

SEQ ID NO: 989 - DNA, 113 bp, Artificial Sequence
  PCR amplified product
gcctaacatggcgtgtaggagctatgtcagggttccagcatgccttgacatgcctcctacacgatccaacatgttccgcaacccctgagcacagctcagcattctacagaatg

SEQ ID NO: 990 - DNA, 110 bp, Artificial Sequence
  PCR amplified product
ccagacatgagcaaacagcaacagaggttagacaggtggaagtccaggggcgatggaggcaatggcatctccaccacagcccctcctgtctgcacatcggtcacgtgttc

SEQ ID NO: 991 - DNA, 116 bp, Artificial Sequence
  PCR amplified product
ctgtctttccaccaaactgggcaccatcaggctgtgatcagggttccaatcacacaaagaccccagcaccctctgtctaaaactcatctccggcctcctgtttcccaaccaaacac

SEQ ID NO: 992 - DNA, 120 bp, Artificial Sequence
  PCR amplified product
gttctagggctgacagaccgagactgtggcagcagacagtggaaacggtggcaaaaagggggcagatgaggaggaaggggagagaacacaacctaaatccggcagtttgaagacacatac

SEQ ID NO: 993 - DNA, 109 bp, Artificial Sequence
  PCR amplified product
gatgcccagctgctgaggagcaactggtcagtctaaggacagagaagagctactggtcaacacaaattcatcctcatctgggaactaacacgtcagatcacgacaaagg

SEQ ID NO: 994 - DNA, 112 bp, Artificial Sequence
  PCR amplified product
cagttcctcatgtacagtccgtatggacaaaaggaaacgtcagccaggctgctggagcagcccctttggtgcctaagtttccctagccgtcagcacaggagggatgatcaac

SEQ ID NO: 995 - DNA, 108 bp, Artificial Sequence
  PCR amplified product
ccttcatgcctgcttgggaagttgtgacttcagccataccgagagatagttggtgggtggagctcagggaggtgtgaactcagggatggattcatatcataccagtag

SEQ ID NO: 996 - DNA, 97 bp, Artificial Sequence
  PCR amplified product
caggcattgtatgaagttcctgggatacagcagaaaactggaagaaatacgatggaattctagcattgtaaagacagggctgaatgtatcatgtgcc

SEQ ID NO: 997 - DNA, 109 bp, Artificial Sequence
  PCR amplified product
catgaccttcttagagaccagggtctcttgaaatcatcacccagcctacgagtcactggctgaggtcacctgacagtgagtcactggcagctagtttgtagggaagaag

SEQ ID NO: 998 - DNA, 111 bp, Artificial Sequence
  PCR amplified product
ctgagtccgaattcaagccaaggctctctcttcaccagcaatacggactcctgaatagagtctgcatcattctctctgcagaatgctcggtggatgtttagagaagtcacc

SEQ ID NO: 999 - DNA, 124 bp, Artificial Sequence
  PCR amplified product
ggctttgggacaagattccttgatcttgggaatgctgagaaccaaataaccagcatcattgtggaccaagcatcccagccccaaacacagtgagtattgactctggatctctacaggtgctctc

SEQ ID NO: 1000 - DNA, 124 bp, Artificial Sequence
  PCR amplified product
cgttagcacaacccatggcgtttcggggtaacagatgccacagcaaaaagcccatgctggttaaggaagatgatactggcgagagtgtctccaaatctttctagcatcttttcagttgatgaag

SEQ ID NO: 1001 - DNA, 103 bp, Artificial Sequence
  PCR amplified product
cccatcctccttgcatgggccttgcatgtcaccaaaaggctccccacctccaggaaggagagagaacatgcctgcaatcacacagccaatgcaataggcacac

SEQ ID NO: 1002 - DNA, 119 bp, Artificial Sequence
  PCR amplified product
ggctgtcttctttgtctcctgtcctctgctaacctgtcctacgacacaaaataaaccttctcacagcttttgggtgtatgaactgcccacagggagtttcctctagacagaaagaactg

SEQ ID NO: 1003 - DNA, 116 bp, Artificial Sequence
  PCR amplified product
gcagttagggaaggttcccagaggctgctagtctgaagactcctgggacctcctgatgtcttttaagcccacacattgtggcccagtgactgatttagcactgctctgtttcactg

SEQ ID NO: 1004 - DNA, 108 bp, Artificial Sequence
  PCR amplified product
caagccacaaactgtagggcagtcgcaacaataccacaaggataaacttagggcaaaattcagaaaagaaattgtggtaacagacttgggacattgttcaggagtcag

SEQ ID NO: 1005 - DNA, 120 bp, Artificial Sequence
  PCR amplified product
gtgtgattactcactaatccctttcccctctcacttttgaccagacccatatgttgaactccagaatgacttgtggatggaggcttgaacttggagcatttggttggaaagccactcaag

SEQ ID NO: 1006 - DNA, 123 bp, Artificial Sequence
  PCR amplified product
ctgtcattgtaacgtttcccaatttgctgtcctaaggaatccaaccatccgattttgtcagtcaggtaaggcctttctacattcccaatcgcataccaaatgcaagccagccagtgagcaatc

SEQ ID NO: 1007 - DNA, 126 bp, Artificial Sequence
  PCR amplified product
cttgtgacttacccttacgcaacctggtgggcacccactttctgtctcatctgaaggctgactggctctgcccctcacaggcgggggccaaggacaccagatctcccgagggatgtcttctcctag

SEQ ID NO: 1008 - DNA, 120 bp, Artificial Sequence
  PCR amplified product
caggttagtagtaccatggcaacagctgtgtactgcaaagatggtggaagatagtttcctaaaacataaggatcttctctttccacatcctctcttttcccaaaatacattcaggcttgc

SEQ ID NO: 1009 - DNA, 119 bp, Artificial Sequence
  PCR amplified product
gttccgtccgattcttccctcatattgtgcgtgctcaagccacaatacctagaatcctgagcattgtaagtgtttagtaaacacctgcttccaaacagtgggtagatatctccaagatg

SEQ ID NO: 1010 - DNA, 117 bp, Artificial Sequence
  PCR amplified product
ctgattctatgggcagcgcctggcgtttgtttgcttgaaagccctgactgatggggttagacaattatgaccttggttcctaaagagcaaagtgcttgcttggaatgttgaattccg

SEQ ID NO: 1011 - DNA, 116 bp, Artificial Sequence
  PCR amplified product
ctccaatactgcacaatccgccctcactcatttgctccgttgcctgtcgaaagcacagagcgtaattactaaagttaagaaaacatccctgtaattagccaggcaactctaagcac

SEQ ID NO: 1012 - DNA, 123 bp, Artificial Sequence
  PCR amplified product
ctttcgtagacagcagccagaataaagtctaatattccggctggggaaacagacacaagcaaacagtgannnnnnnnnncctgttgattttattttcctttgtgggcatctgttaacctaggg

SEQ ID NO: 1013 - DNA, 134 bp, Artificial Sequence
  PCR amplified product
cagctccacaactagtaggtacattgactcaacatagagaaaacgggaaatgtaaagtctgagggttgtgtgtttgggagaggtggggtgggggtgtctcatttttaaaatacacgttcaagttttggacaagg

SEQ ID NO: 1014 - DNA, 114 bp, Artificial Sequence
  PCR amplified product
caaccacattgatgtgagctcctcatacatcttcagccaaggcacacagaaaaaggaaatgcctgacaaacaacccttctgagtgaagaatgatgggatctaatgccaggtgag

SEQ ID NO: 1015 - DNA, 120 bp, Artificial Sequence
  PCR amplified product
ctgcagcacctgtcatgggggactcgtgctctgtgtcacgtagtagctgctcaataattcctcctggaggggattgctgatggagtccttgcttttccctggaaacagtggattgtcaag

SEQ ID NO: 1016 - DNA, 105 bp, Artificial Sequence
  PCR amplified product
ctttgctcagaccaacacgtctgagttgccatgcattcgaagagtgggtgccatggttcccagcagacatgtggctcaggacttggccagatatgcctgggtacc

SEQ ID NO: 1017 - DNA, 132 bp, Artificial Sequence
  PCR amplified product
cagctcaggatggaaaaggcaaattgggagcggggccagagctaggagaggtacaggatggagagaagttgctgggaagggaggccgaggtaaggccgggccggtgaaaatgggaacatgttactcaacctc

SEQ ID NO: 1018 - DNA, 120 bp, Artificial Sequence
  PCR amplified product
cagagcaagagggatgggactgagtcctggtgctgagactctggggagggacagactactttgtgattactcaaaaggcgaggaggggtgatgaaataagccatttctaaactgtgcttc

SEQ ID NO: 1019 - DNA, 120 bp, Artificial Sequence
  PCR amplified product
gagggaccaaactatgaaggaatgctgatgagccttagaattggtttctgtctgcgtcccacttcttgcaaacccctagttattaaacactgtaattcttgcatctataggagccctttc

SEQ ID NO: 1020 - DNA, 114 bp, Artificial Sequence
  PCR amplified product
gaagtgtcaacagcatagcccaggaagattctggagatacctaaattaaagcagcatgagtgtaggggagcccctgtttctcaaagccgggggtgccaaatgttatggtggaag

SEQ ID NO: 1021 - DNA, 116 bp, Artificial Sequence
  PCR amplified product
gcatccacacgtgatgtgcgtcaagcttctgaagctacgctcctgaggaaggctttgtgcagctcagacttccccaccatctgctaaccatgccctgcgatggtttccatcctaag

SEQ ID NO: 1022 - DNA, 127 bp, Artificial Sequence
  PCR amplified product
ctaacctattgccagctgcacacaggagttagaaaaaggttacaattcatcccacccgatttgagatttttccagttaaagacatggcgaggtagaaagaccaagtcccttgtgatgttagatggtc

SEQ ID NO: 1023 - DNA, 106 bp, Artificial Sequence
  PCR amplified product
ggactgcagctagtatggcccccatagctattgaaatgcccgagacacgtcagtgtctaaacatctctcagaccaccccttccacttggatgaacaacagccatcc

SEQ ID NO: 1024 - DNA, 116 bp, Artificial Sequence
  PCR amplified product
ctttgttaagctcactttgcaacataagaggaattcagacctcatagggatgtgagctacatagttattcaccatataccctcaggaagaagtagagctaaactgagaagcactac

SEQ ID NO: 1025 - DNA, 100 bp, Artificial Sequence
  PCR amplified product
caggatgtgaccactggctcattcctaatgtttcaggtgggtaacaaatatttgctttttccggggagttgaccacacaccctcttccaattccttcaac

SEQ ID NO: 1026 - DNA, 109 bp, Artificial Sequence
  PCR amplified product
ccacagacagttctagagggtgtgcttgactttggaaacccagtttaactggcttctgcttgaatcatcactccattaacatcatcattccagaaagtcgttgctcaag

SEQ ID NO: 1027 - DNA, 106 bp, Artificial Sequence
  PCR amplified product
gaagtttctgggacacaaagggctttctggctttgtcaagctggtcttgagagatgaaacaggcaccccgcgccatgtgctaacagtcacttttaccaagttttcc

SEQ ID NO: 1028 - DNA, 114 bp, Artificial Sequence
  PCR amplified product
gtcattgctggaaattgattcatagagtttcagagcttctctagaaggcctcagccatgtccttaaaagttgcataaaacttttggcatatgagtgattcagggatcctgacac

SEQ ID NO: 1029 - DNA, 113 bp, Artificial Sequence
  PCR amplified product
cttcttctcttcaagggtagacatggtggacgtggatgcaggatagcaggcatcaggcagatgtgaatggcatggaaaaccaggctcctggagacattgtgagtgctgagtcc

SEQ ID NO: 1030 - DNA, 120 bp, Artificial Sequence
  PCR amplified product
cgtcaacacggattacattctgaaaccatggatgcacacctcacattcctggagtcatctaacactagcatcagcaggtggtcttgacatggtcctggacccattctgtgtcactgaatc

SEQ ID NO: 1031 - DNA, 105 bp, Artificial Sequence
  PCR amplified product
caccagccagcattcagcacagcagctttgaggtggcgatcgctatttccccaactcaatgaactaaagtactagaagaaaatctcccacaactcacaaaggaag

SEQ ID NO: 1032 - DNA, 120 bp, Artificial Sequence
  PCR amplified product
ctccagcctgtctgtaggtaggaaaaactactcctggaagctcacctcagtgaatgcacctcagagtccaagagctgccgcgaatacagggcctggtggctgctatactgtgcgtctcag

SEQ ID NO: 1033 - DNA, 111 bp, Artificial Sequence
  PCR amplified product
gtaagccctgtggttctggcacggtatccatggtccaaccagagggctgagaggtctcacactggggcataagcctggcccaggccacacagccagattggcaagctacag

SEQ ID NO: 1034 - DNA, 116 bp, Artificial Sequence
  PCR amplified product
gctcaatgacaatgctgtccactacagcaaaaccgagtgttctcctaggcctgctgccaccctgggcacatagtgagaacacgcccacttctgctgtggacattgaggcacagacc

SEQ ID NO: 1035 - DNA, 109 bp, Artificial Sequence
  PCR amplified product
gctctgggtcatcttcccgacctgaaacaggccaagatatgaaggccctgagccaggaaaacctactaagggatccctgatcccaagtcccctcagagtgacccaagac

SEQ ID NO: 1036 - DNA, 106 bp, Artificial Sequence
  PCR amplified product
cacctctggagggagtgccagagcaaacatgggagccaagcagcccagatgtggtgggtggggagactcaaatttagcaggattgagggtcagaacttgttctccc

SEQ ID NO: 1037 - DNA, 114 bp, Artificial Sequence
  PCR amplified product
gaccagacctctaaacaccgcccagatcccagagtaaaggcagataaggcagtagttaagaagtaggaagaagtaaaggcagctaccccagagaagctaaggtcgggagaggtg

SEQ ID NO: 1038 - DNA, 116 bp, Artificial Sequence
  PCR amplified product
gcatacgaattcccaaatcctggcgcagaaaggaagaaggttcccctttaatgcggttgtctggtgccaccgcacagcctggtaaataagtgtgctaggatttgggacgtgtccac

SEQ ID NO: 1039 - DNA, 119 bp, Artificial Sequence
  PCR amplified product
gtttggagggatggaaatggagcaggaggaggctggagaaactaggaaggcccagaccacacaggcctgctgagacacattgcggagttttggccttttccctaagagtaacccatgtg

SEQ ID NO: 1040 - DNA, 116 bp, Artificial Sequence
  PCR amplified product
ctgtgaggatgatggacaggagggcagcaggggacacgcattagctcccctgtcatccctctgccagcacctcccaagagcagtttgtgctaggtgtgggaagcacaggaacagag

SEQ ID NO: 1041 - DNA, 106 bp, Artificial Sequence
  PCR amplified product
cagtcatcttccaagttgcacgtggacggattcaatgatcccagctatcccctcccgaaattaaactgatgagcaaatgaaatgcaagcacagagtttgtggaagg

SEQ ID NO: 1042 - DNA, 111 bp, Artificial Sequence
  PCR amplified product
gagagcagagggcttctggtaaatgaggacactcccattctggcagtgtcaggagagtggtttcggggagcgtgggtagtagggagagcagtggggagggctgtaatgctg

SEQ ID NO: 1043 - DNA, 134 bp, Artificial Sequence
  PCR amplified product
cagtggattagcctaaacgcggtctgcagccactattcagactgaatgaggccacttttccccccagaaggatgtgtgtgcatggggtcacagtcctgcgagggagacctggccgcactgcaaagagaacagag

SEQ ID NO: 1044 - DNA, 108 bp, Artificial Sequence
  PCR amplified product
cttcatgctctcatcaaaccggtaacttcctggttcttgccatgcagcctacaaatgtgtctccagccctcgctgctgtgtgggtttgccatctcaatcagagaactg

SEQ ID NO: 1045 - DNA, 118 bp, Artificial Sequence
  PCR amplified product
gttgacaagtagtgggttcccagtaggcattgctgatgcatgagtgcacgactaaattactgtgcccctttgtggcgtgccccaacgtgaaatgctagggcactgaatttcactgtac

SEQ ID NO: 1046 - DNA, 110 bp, Artificial Sequence
  PCR amplified product
gtgaatgacatgggtgaggggtgcagggcatggtgaatgcagaacggtgccaggtaaaggaagcgcctgctgtgccatgcaggtaagtttagctacaggtagaaacaagc

SEQ ID NO: 1047 - DNA, 123 bp, Artificial Sequence
  PCR amplified product
cacagacagctgctcagggagccccaactgtgtaaacctttgccagtggattctgaggagaaccccgatatcaagcagattaacgccgggctactttggttaaagcacaagtggaggatccag

SEQ ID NO: 1048 - DNA, 112 bp, Artificial Sequence
  PCR amplified product
gagcaaagctaatccattcccaggtggcacatagcagcacagaagccatctgctgcttgcatccaccctgggggcctcacctgctcaccacagctacttttccaaggactac

SEQ ID NO: 1049 - DNA, 107 bp, Artificial Sequence
  PCR amplified product
gcgcttgtctcttttctggtcaattcctgtgttctgcacatactgcagaagattttctgcccactgggaaggctctgtgtctatgttggccttcattcagcccattc

SEQ ID NO: 1050 - DNA, 126 bp, Artificial Sequence
  PCR amplified product
gttggtcctccatagaagccaaataatccacttgtctggtattcaccactgcccaggaaaagaaatgagtgaaagaggcacctggtgaggtccattgcagggaggcaggaaggcacagaactcatc

SEQ ID NO: 1051 - DNA, 111 bp, Artificial Sequence
  PCR amplified product
gtatccccttcacttctggtccctactcagctcttgttcctcagcctcctgctctgggtgcacctgctccccagccccatgcactgactcttgctatggtcaccagtgttc

SEQ ID NO: 1052 - DNA, 109 bp, Artificial Sequence
  PCR amplified product
gaacatgggatgaactcagcacacacactttactcaggttggaagcagaacgaaaacccaacaccactggcgggcgatgtggaggggcagggaacttgagaacaacagc

SEQ ID NO: 1053 - DNA, 112 bp, Artificial Sequence
  PCR amplified product
gaggtgaagatcattctaacctgagaagcaatggaaagatttgggcatgggccctaaggattccactgaattctgtgctagagtatcattttccaaatgtcttcctcatagg

SEQ ID NO: 1054 - DNA, 115 bp, Artificial Sequence
  PCR amplified product
ccaaaaccattcacttaggggaatttcaaactttggtgctaaagcttccaaataatcagcatcaccattcaccaaggagcagaggagttcggtcttgctgactgccagttaaggg

SEQ ID NO: 1055 - DNA, 114 bp, Artificial Sequence
  PCR amplified product
gccatcttccaggttttccacaacccacaaaggtctttcaggtgggtataatttggggttacttgttaagatggagttacagcacagcttcattggcaaagacaatgggatctc

SEQ ID NO: 1056 - DNA, 131 bp, Artificial Sequence
  PCR amplified product
gaagagcctgtttcagtggcccacctacaggagggatcagagcacatccatggagctgagtgccgcccggtgttactggaaagcagagagggaaggacagagaattacagcagggtgtatgtaggcactac

SEQ ID NO: 1057 - DNA, 135 bp, Artificial Sequence
  PCR amplified product
gtagaagaaagatccaccccctccttcaagctgatctccactggggacggtccacatatttctctgctttgcatttttgctgttgcttggttggttttttgttttacattattactggaagaatgcttcttgcag

SEQ ID NO: 1058 - DNA, 119 bp, Artificial Sequence
  PCR amplified product
cagctgggaatgtgatacctctctgcagtagcaacactgtgaaaagatgccaccttgccatctctacaggtggctggaattgggaacatcactttgatctggcatagttgctgggactg

SEQ ID NO: 1059 - DNA, 114 bp, Artificial Sequence
  PCR amplified product
caacagaaagaatagcttgccatctttaggactggaggaatggcaaagctctttccctttcagcctccaatggggggacctgggcatttgtagcctgttcaaagaaaccaagag

SEQ ID NO: 1060 - DNA, 122 bp, Artificial Sequence
  PCR amplified product
gtgctactttcatggctaggaatgaagttgttgggtttctctttcagagctacccctaaaggcattcactttatattctctgaagagaaccagctaaccaggcggaacatccactaagaatc

SEQ ID NO: 1061 - DNA, 114 bp, Artificial Sequence
  PCR amplified product
gttacccacaaactcaacgggtgggttctgagaagcagatgagccatgaagagcatcaaacaagcattactgctctggccaccaccagggtcacctttgaaccgtaagaaagag

SEQ ID NO: 1062 - DNA, 132 bp, Artificial Sequence
  PCR amplified product
cttttcagcagacttttggctttaagagttcttaccaaaaagattgctaaagtggaatgagaggggctcaaagnnnnnnnnnatttcattataagtgctgtcccatcttatccaattgtcttcttcctcatc

SEQ ID NO: 1063 - DNA, 115 bp, Artificial Sequence
  PCR amplified product
gtcatgacaacttctgtccccttcacacagaccgcttaccggaaacaaaccttgaactccccctgcttaagactgaagcctctgtccatctgaccttcccttggcctgggaagtg

SEQ ID NO: 1064 - DNA, 112 bp, Artificial Sequence
  PCR amplified product
cccaacagggacatgtccgtcacggtggtgtgttacatcatgtcagatggcaattgaatgcgctgttaacataagctgacaggaaggctctagcaatctgcttatctggatg

SEQ ID NO: 1065 - DNA, 120 bp, Artificial Sequence
  PCR amplified product
ccagtttcatagacatcttgcaaaggaaaagattctttcctttgcaagatactatgaaaagtattcataggagaaggcatctgcaggaatccatagaccctccaaaacatccccagcttg

SEQ ID NO: 1066 - DNA, 111 bp, Artificial Sequence
  PCR amplified product
gggatgtgttgcacaaaagcagggctcagtctgtcaactctttaggttctgagggggccagatgctccccgttgttattccaggcccggcggctagaaagtcagagtgttg

SEQ ID NO: 1067 - DNA, 114 bp, Artificial Sequence
  PCR amplified product
ggttctcctgacctctcctccagttgtgtttctgttcccaaggtggtgcctcggtgctaatacctcgtaatttttctgtcaaacctttccagtggctgagtacaattgatgaac

SEQ ID NO: 1068 - DNA, 115 bp, Artificial Sequence
  PCR amplified product
cagctctaccaaccacagcaggagacagaagtggtaaagtttcttcgctcatgcagacgagagccatgggcacggggccggcaccaggaagaagcgcgttcattcattgggtgtg

SEQ ID NO: 1069 - DNA, 116 bp, Artificial Sequence
  PCR amplified product
cttcggtctgtgttgaagggacagcccccacccgctgttcttctctgggctggcgctgagctttgcctgtgcatggaatcacccagagctgtcggatgggttgtggatctcatgtc

SEQ ID NO: 1070 - DNA, 112 bp, Artificial Sequence
  PCR amplified product
ctgtggcttgatttcttccccctcccatgaatgcggaggaagccgactttgagagatgaatgaaccatgtcagtcctgtcttgagaagcccctcgtctagcaaaggacacag

SEQ ID NO: 1071 - DNA, 109 bp, Artificial Sequence
  PCR amplified product
gtaccagtcaggttatgccgtattcagctgagtaacaaacatcctcacaacttccaagccaggcaggagcccagggagagttggaacatgcaagattcttctagagggc

SEQ ID NO: 1072 - DNA, 116 bp, Artificial Sequence
  PCR amplified product
gatgccaaaactaaactctcctctcagaggtccaagaaagcacattagttttaagataaatcagagttccatttctgtccttggacgtgttggcaagggggtgggtgtttctggac

SEQ ID NO: 1073 - DNA, 104 bp, Artificial Sequence
  PCR amplified product
caagcaggagaggcatgcatttgctcttggaagaactttaggaacagtcttatgatgggggctgcttcccacccacagctttttgcatgatgtatctgtagagg

SEQ ID NO: 1074 - DNA, 103 bp, Artificial Sequence
  PCR amplified product
ggccacagcaatgttggggagtttcactctggctaacaggttggtttctaactcaagtttccatttaacctcataactgaaaggggtgcctattcaattcagg

SEQ ID NO: 1075 - DNA, 105 bp, Artificial Sequence
  PCR amplified product
cacctcagagccaatagcccagaacagctgtttggacatggattgttctctcttttgcttctgatgtggaactttctttccagcagggatctctggtgacttctg

SEQ ID NO: 1076 - DNA, 104 bp, Artificial Sequence
  PCR amplified product
cagcctgaaacaacaacggatggtcaacgcaagggtaatccaccagaggaacactgagcggagctgtaccgccccagccacatcagctactgcctttcttggtc

SEQ ID NO: 1077 - DNA, 114 bp, Artificial Sequence
  PCR amplified product
gaatgcagcttgatgatcccaaataaccaggagaggaagtgtcacagggtgaggacaatgcagaaactacccacttcttcctgtgcccttgatcctcggaggtgtttggctctc

SEQ ID NO: 1078 - DNA, 114 bp, Artificial Sequence
  PCR amplified product
gacaatggaggaagtaggctggactgccagaggcttggattttactgacatctgatcttagggcatcacacatgggttggccatttgaaagaattccaaaagcctatgccattg

SEQ ID NO: 1079 - DNA, 140 bp, Artificial Sequence
  PCR amplified product
gggttgctgggaaacagcatttaagaccttgttaacaatatgcttgacaacagcaacaaccaggcataacataactaatagtagcatgctctaatcagccccctatagagacaagtccaggttatgagaaaagagtccac

SEQ ID NO: 1080 - DNA, 115 bp, Artificial Sequence
  PCR amplified product
ccaaggaagaaacccatgcataaggcgatgaaaccagtatccctgtgacctctaggccgttcgctcttagacaggcaggtcctttgggtgatggccagtatgtaaacactgaaac

SEQ ID NO: 1081 - DNA, 120 bp, Artificial Sequence
  PCR amplified product
gcaagtttactatcatcaagcaaaaaactgactcagaacggaacgtgacctttggggatgcgcggagaggggtccaaagtagaattctagataaacatacacctctcagagacagagttc

SEQ ID NO: 1082 - DNA, 112 bp, Artificial Sequence
  PCR amplified product
gaaacaaaccagtccaacccagcagatatgggtggaaatggggtgagtagaggaggggttatggctacaaaatctaagcagaagacactggaccccaacaagcactttgctc

SEQ ID NO: 1083 - DNA, 115 bp, Artificial Sequence
  PCR amplified product
gaaaccaccacctaaagagggtacaagaaagaacaggatgagaatgcgggggagaacgcgtgtgcacctgaccacacagactataccacaggccctgtttgaaacaaaacaacag

SEQ ID NO: 1084 - DNA, 135 bp, Artificial Sequence
  PCR amplified product
gagtgagcagccagaacgcccctgacaacagctccctccagactgggtaccgcccccacgcccggcgcatcctgggagttgtagtcctgtagccctgcaagccactggcttagagcagttaagagactacaactc

SEQ ID NO: 1085 - DNA, 136 bp, Artificial Sequence
  PCR amplified product
ctagcactctccccaaacctctctcgcacgcggggactgagcacggcccgaaaagccgaggacagccggactcaccctgtagttatagtagtgcgtctgcacaagatgccggtggcgcgacttgtggttgccgaag

SEQ ID NO: 1086 - DNA, 117 bp, Artificial Sequence
  PCR amplified product
cacactgtttggttcacaggactctgttacccattggggacctcttggccattattaacacaggccaacaggactaaaagtttgtatcagtccttcccaaaacattagggaaggttg

SEQ ID NO: 1087 - DNA, 111 bp, Artificial Sequence
  PCR amplified product
gtgctcactgtcaacccggccagcagaggccatgcataggtggccaggtgcgactacctgtgttccagcaagtagatggaaaaggaacactgtcgctttctcagggctgac

SEQ ID NO: 1088 - DNA, 132 bp, Artificial Sequence
  PCR amplified product
ctaagtatgcacttttgtgagcacttgttctaaattattgccattacatatccacactggaattgaaaaataacccagctcaattcatcggccaaagacacccagcctccatggtcagagtatcttctatgc

SEQ ID NO: 1089 - DNA, 104 bp, Artificial Sequence
  PCR amplified product
ctgtggcatgaacagaatggagagagacttgggatcttaccgggagacaagatcatacccaccaacccaacaaatgaggccacaggcatgcaagtgagatacac

SEQ ID NO: 1090 - DNA, 115 bp, Artificial Sequence
  PCR amplified product
gattcccagtgtgaactccgtgtcagactctgctttaggaggagacagatcctatttcagggctgggcacacctaaagatggagcctggcgaggagcatgacttagggtgagaac

SEQ ID NO: 1091 - DNA, 114 bp, Artificial Sequence
  PCR amplified product
cctcattgaggacttcaggtcgttgcactgtactatacaggggattcgtgtggaatgagttgattgctgctgctcttgccccacaacacacacacgcacgtgcataactgaaag

SEQ ID NO: 1092 - DNA, 117 bp, Artificial Sequence
  PCR amplified product
caacgcgaccaacagtgccacactggagtgccttctgggatgagcagaatgcctttagaccagtcacagtgtggctgcttccgtccaaatggcgctcggcatgggcatggtttgatg

SEQ ID NO: 1093 - DNA, 110 bp, Artificial Sequence
  PCR amplified product
ctatggataacaagcagaggtaacaacccacttttcatcagcatattcttttttccagaacacaatgcaattactgagtgtcagcttcatcgtccagttaccattcagtc

SEQ ID NO: 1094 - DNA, 119 bp, Artificial Sequence
  PCR amplified product
gtcaggatccttgcaaagcaagataagagtaaatcagatcaagtgcatggtgagatatggctgtatgagagtttgcagagatatttttcttttcccttccagaccccatttgagtaatg

SEQ ID NO: 1095 - DNA, 132 bp, Artificial Sequence
  PCR amplified product
gaacatcactctggaaagccagggagattttgtgcaaatctgatgaggcaatacattgggaaattaaacatggtaatgactctctggtgaactgatatacgactctccttttacctaccctctgtcaacctc

SEQ ID NO: 1096 - DNA, 132 bp, Artificial Sequence
  PCR amplified product
gatgacaacagactattcggaaggtactttgtctcagagttctctgaaatgattagctatgtcttacttttacccgctactgaagtgaagattgtaggacaccttctcaggcccccactagcccaaatggag

SEQ ID NO: 1097 - DNA, 105 bp, Artificial Sequence
  PCR amplified product
gttccctcctgtctttacgaacagctgtgtctcaagaggtcactgagggagcactggctcttctcacagccagctctctcttgaagcttcttcttagtgtagatg

SEQ ID NO: 1098 - DNA, 118 bp, Artificial Sequence
  PCR amplified product
gtggaaaagaaaccaggcccattttcagccagtctgaggaggaaagaggtccctgaagaggcctggggtttgactgctgggacctagtggggcaagtggcatggattagctgaagttg

SEQ ID NO: 1099 - DNA, 101 bp, Artificial Sequence
  PCR amplified product
ggcaacagctttgaaaaccagtccattcttgtcctgaaggtaaaagcccacaatgtcaacctgtagactctaccttgagggcctctggcataagattgatc

SEQ ID NO: 1100 - DNA, 112 bp, Artificial Sequence
  PCR amplified product
cttcacaggagccactggaacagaattcaagcaactcaggaccctgcagttccttgggtcatgaaggaagtaggagatgtgaatgcagagcccatcagaaagcaattcttag

SEQ ID NO: 1101 - DNA, 118 bp, Artificial Sequence
  PCR amplified product
gtagcccagagacagtagctgtctgagttcaaacctcggtttggggcccgattctttttctcagttcagcactggaggttctcggcacccagccatctggattgacactggaattcac

SEQ ID NO: 1102 - DNA, 124 bp, Artificial Sequence
  PCR amplified product
catggacatcttcatagagctcgtcacaaaccctgatgggtttgcttttacccacagcatggtgagggaacctgggaaggatggaaggagggggtcagctctagggggatggagaaaaggtcac

SEQ ID NO: 1103 - DNA, 104 bp, Artificial Sequence
  PCR amplified product
caacaggaactggaagtcgggttttggaggtatggcaacctgctgtcctggggcagggttggagaagaggtgttggcccatggcaggttctcccactcagtcag

SEQ ID NO: 1104 - DNA, 115 bp, Artificial Sequence
  PCR amplified product
ctagccctgccctgaagggagcacaacatgaagaaatgcctctgaactctttccccgagagctaggacctgaaatctgccctctggggaggccagggcaatagtttcaagtgtcc

SEQ ID NO: 1105 - DNA, 124 bp, Artificial Sequence
  PCR amplified product
cagaagcagcaaatgcaagctgaagtctaacttttgcaaggaaatcagggctccttctggctcctctgatatctaccctcatgacagaattccaagcaagagggccactttcgtttccaatcac

SEQ ID NO: 1106 - DNA, 129 bp, Artificial Sequence
  PCR amplified product
gatggtggcttgcttttcccatttgtgaagtctggtggtaacagtacccagacagggaagtgaacaaccctatagtatagtgaccggatttagcagggccggatcgccaccatctttaggaaagtcagg

SEQ ID NO: 1107 - DNA, 126 bp, Artificial Sequence
  PCR amplified product
cttctctgttaactctgtgccttgattgcttaagacaagacacatgtaaaccccatgattattgccatttttttggactttgcaaagactctgccttcaaacataaagctgctgggctcgttttgg

SEQ ID NO: 1108 - DNA, 110 bp, Artificial Sequence
  PCR amplified product
gtcaaactccagggacaggcagggcctatgcagtgcgaggcgagaaccgtccgatcggagcacctgttctatgtggggatcagcttttccttccttaagacaaacaatag

SEQ ID NO: 1109 - DNA, 113 bp, Artificial Sequence
  PCR amplified product
gcatcctctgaagaggcgtgtttgtgagcactccatccacggggcgggtggccttcttgtacttttgatgtttatacattctgatgatgtgaccctgtgacctgttcactttc

SEQ ID NO: 1110 - DNA, 109 bp, Artificial Sequence
  PCR amplified product
gaccacataaccctagagcagcaaagaatggtgcgatcgtaaaggaagaacccatatttgctttgggnnnnnnnncccctagctatttgggtgatgttagagtaaactg

SEQ ID NO: 1111 - DNA, 109 bp, Artificial Sequence
  PCR amplified product
gaatgtcaagtggatgtccagaccttcatctgacatagttagcttagcaaaaacaaaagtaagatctttgttcagagggaggaaattccatgccatggatgtttctggc

SEQ ID NO: 1112 - DNA, 118 bp, Artificial Sequence
  PCR amplified product
ggttagcaaagccttctcctgaatcctttcctattctcaatggcatgatatgtcaagaacgtcttttgagcctgtttgatccagtgatgttcaaatgtgcaacaaggaacttagtctg

SEQ ID NO: 1113 - DNA, 106 bp, Artificial Sequence
  PCR amplified product
gacctgtgtttagatgtgctgtcacttctgaaggaagtcatccgagcttaaacttatgggatctcacaaggggcctgcagtatctccttaaggaccaccataagtc

SEQ ID NO: 1114 - DNA, 118 bp, Artificial Sequence
  PCR amplified product
cgcttactggagactgtgctcaagaaaaagccaagaggtaatcttcggcagctgctgtgatatctgcatattttaattttttccatctatttaaagcctgccaagttgcctaagagtc

SEQ ID NO: 1115 - DNA, 112 bp, Artificial Sequence
  PCR amplified product
gtagctgttgtggagtagcagtgggtgaggtgtatagagatccattcatccatgcagcaaaacacttgactggcttgagatgtgacatgcggagcaaaaggcaggacattgc

SEQ ID NO: 1116 - DNA, 107 bp, Artificial Sequence
  PCR amplified product
gaatctgaggctcagggcagcagaagagggctcttggagaagagatgacagttggctgaagtcgtcaacagagggagctgggaggctgctaacccctttgatttcag

SEQ ID NO: 1117 - DNA, 107 bp, Artificial Sequence
  PCR amplified product
ggcctccaaagtctttgggggctgctcctcaattcagtcctataaagtgcatggcatttggccctcggaagcccctcaaggctgagaggctttacttgccacatagg

SEQ ID NO: 1118 - DNA, 114 bp, Artificial Sequence
  PCR amplified product
cccaaaggagatgaacaggagagaagagaaaaggccatctgcatcctccccatgagctccagagagcacgagtggtggtgagtgactttcatccaccctgatcccacctcacag

SEQ ID NO: 1119 - DNA, 117 bp, Artificial Sequence
  PCR amplified product
gtggaaaagccatcactccctgcagaggacctagaggacaggaaccctgaccgacctggacagggttcctacaggggaaggcagggcaggaggccttgctttgaaagagcactaccg

SEQ ID NO: 1120 - DNA, 126 bp, Artificial Sequence
  PCR amplified product
caaaggcatagggacctgccccaggtgggtgcttttcacaattctgagtccccttcagctaaacacaggacctccttgggttctgtcctaggctgaggcctctacttccttccaactccacccttg

SEQ ID NO: 1121 - DNA, 111 bp, Artificial Sequence
  PCR amplified product
gctcagcactaacccttcccccagtaaagactcactcagcagaaacagttctccgtaaggtaaaggacacagcacagaaatggaagcaaatcctagaagaggtcaaggaag

SEQ ID NO: 1122 - DNA, 107 bp, Artificial Sequence
  PCR amplified product
gaatgtccacaccaggggcccaatcttcattctaatgagaagtccacattttagagatgttgtaggtgcctgcccagtctggctgaggccactgaacagcacgatgg

SEQ ID NO: 1123 - DNA, 119 bp, Artificial Sequence
  PCR amplified product
cagagtctcagccacaggtggagacagctttacagatgagacgaaccacttctcttgtggattttccacggtgacgagtcagctgtatcactatcatatcccacagatgcagtctgcac

SEQ ID NO: 1124 - DNA, 116 bp, Artificial Sequence
  PCR amplified product
ccaacaactcaatgacattccagcaacttcgaagagaaagtcccgtctccccaggtctgccttcctgccttccccaattcagatcccacagctcacggaatggctctgtgtcaaac

SEQ ID NO: 1125 - DNA, 117 bp, Artificial Sequence
  PCR amplified product
ctgttccatggttgaccccaagaatcctcaccaacagtcgacattacacttgaggctaagtgccacatgagggggctcccatgctccaccagccctcggggtgtcactggtaagtgc

SEQ ID NO: 1126 - DNA, 109 bp, Artificial Sequence
  PCR amplified product
cttctcagaaatcttcttacgctcccacaaacctcccaggtcacctcgagggaggcaatggaactactcacgaaagaataattggatttcccagttttcctaccagagg

SEQ ID NO: 1127 - DNA, 114 bp, Artificial Sequence
  PCR amplified product
ctgttaacgtgctcgtgtcccaccacgcaacgggtgcttccacacaagcagccaacgcagggggggctgcaaaacctggaaaccacacaatgcatgccttgaaatgagggtagc

SEQ ID NO: 1128 - DNA, 124 bp, Artificial Sequence
  PCR amplified product
gcacatgcctgtcacacccatttccccatgtgagggaaggaatcggcctggaaattcccaatttctacataaagttcactatatttaggaggaaaaaatgtgactcctgttgactagtggacag

SEQ ID NO: 1129 - DNA, 117 bp, Artificial Sequence
  PCR amplified product
ggtaactcttggagcatggatgccacacttatgacaagtgagcagtgattctcagcacagaatgtgatatttttctgttgcacaaagttaaacagtgaccgagtgtccacaattgcc

SEQ ID NO: 1130 - DNA, 116 bp, Artificial Sequence
  PCR amplified product
gtgcagtgctaaaccttggagattctcaggtttgttttgttaagggaggcagtattcccttaccagctcccccagagagcctacatttgtccaggagcttttggggataagctgtg

SEQ ID NO: 1131 - DNA, 113 bp, Artificial Sequence
  PCR amplified product
ggtagggtttggctcagggaggccatagaggggtccattgctacaggttgccctctggcctcgatgcccacctgtaaactgctatcttcaagagtggaacccagagtggttgg

SEQ ID NO: 1132 - DNA, 128 bp, Artificial Sequence
  PCR amplified product
ggttgggtcacttcgatcctgcctgggcccaggtgctagtagggtctttagccttcagctgaaggttctcccctgctcctccaccatctgtttggctttacaacacacacctagtccttggactcttg

SEQ ID NO: 1133 - DNA, 121 bp, Artificial Sequence
  PCR amplified product
ccatttctccttgatttcagcacccaagtgaacatgcactccaaggctctgctgagggtaaacagaaagcaccatcgcaggggtccttcctcctctctctcggaagattcactttctcttc

SEQ ID NO: 1134 - DNA, 108 bp, Artificial Sequence
  PCR amplified product
gaaggtggtacaaggaacctgcaggagactgcagtatcaggtggcagtatcaggaggctgataaatccaggctaatggaaattactattgcacaacaccagagtcttc

SEQ ID NO: 1135 - DNA, 111 bp, Artificial Sequence
  PCR amplified product
cccatgctctgggtctgggtcatgcctcaaccttcttcccagggaagaacaatctttacacagaagtttagataagttcctatgacattagaccacttttggagacttctg

SEQ ID NO: 1136 - DNA, 128 bp, Artificial Sequence
  PCR amplified product
gagtttgggtgtttcttctccattnnnnnnnnnttctccactcttggaacatttcagttgactgggttttcattgaaccccattcccagccttattcctaacatttttgcctggatacatttggtttc

SEQ ID NO: 1137 - DNA, 114 bp, Artificial Sequence
  PCR amplified product
cccaagagtgtcaagtagctttttctaggattgccactgggcccacaggcattctctgaatcactccacacgcttttggggtgggaatcgggccccagtcagacatgaacaaag

SEQ ID NO: 1138 - DNA, 111 bp, Artificial Sequence
  PCR amplified product
ggacgagctagagtttggaatttagctgattaggtagtatgcctgggtggggcgactgggtccctgcctgatttacaattacaagacccctcgctctgacagctcacaacc

SEQ ID NO: 1139 - DNA, 131 bp, Artificial Sequence
  PCR amplified product
gtgttgcatttggcaacaccacagaagctcctcaggtatcacactcctcagaggcaggtggtataatcttgaattgagatcactgaagcacatcagaaaccacacctcccagctgaggtctaaagtgaatc

SEQ ID NO: 1140 - DNA, 116 bp, Artificial Sequence
  PCR amplified product
ctctagctgggcatgagggaagaggtgcagtccttacaaaaggtctcaggtaagaagctggtcttgaaaattctttgtgtaagttcagaattctcaatgcctagtacaaggcctcc

SEQ ID NO: 1141 - DNA, 129 bp, Artificial Sequence
  PCR amplified product
ggtaagaaaatggtccatccccctattccacagaaaggatgctcataactacatgatggatgaaaaagaaaatattaacaaattctgtttgcaaatctaatatactttgtggaatagtctctaatgttg

SEQ ID NO: 1142 - DNA, 106 bp, Artificial Sequence
  PCR amplified product
gtcagggttctttcaaggctcccagtgatgaacaacagtctcctacctcatccatctatcaaagaaaactcaccctcaaggtttacctttcattactggatatacc

SEQ ID NO: 1143 - DNA, 101 bp, Artificial Sequence
  PCR amplified product
gtgctttgttctctttgacacagctatcattctgggacttctgtatacagcctttcctttggtggtcttctggtgctccttggtcttgcatctttcttgag

SEQ ID NO: 1144 - DNA, 105 bp, Artificial Sequence
  PCR amplified product
ccgtaatcattacaatgatggtcccaatctagaggtggaaagttgtttgcctggggtggtgagtaattctctattcaaaatatgaagaaatgtgtagaatagttc

SEQ ID NO: 1145 - DNA, 110 bp, Artificial Sequence
  PCR amplified product
ctcatatgtaaaggaacaacagcttctacctttcttagcctttccctcagcctcttaaaaattatgcctacaattataccagtcacttcagcattagggaggtttaagtc

SEQ ID NO: 1146 - DNA, 113 bp, Artificial Sequence
  PCR amplified product
gaagggatgaattacaaagtggtgtgagaaatgtttgagtgatggaagcttttgttgtctttgtcaaaatgataaaattgtacaataaaaatgtgttgtatatcatgcttcag

SEQ ID NO: 1147 - DNA, 124 bp, Artificial Sequence
  PCR amplified product
gctaagtcaaagaacaagggtggctatcagggtcaaccaagcagcaaggtgccaaggcagtccccaggggttgtttgcagaggatactggcacacttacacacacaactgaggtaacaatagcc

SEQ ID NO: 1148 - DNA, 122 bp, Artificial Sequence
  PCR amplified product
ggtaatgtagataaggtatccctcagcaccctgatcaataaggaatcacttttcacattatattgtttaacaaattctatgctccaactgctccaaattatggatactcatgtggtacagag

SEQ ID NO: 1149 - DNA, 102 bp, Artificial Sequence
  PCR amplified product
caacagcagcatctcatgcatctgaaactctaatagacaagccacaatttctgggagctaactatggcttccaggcctgggtcacttttagaagataaacac

SEQ ID NO: 1150 - DNA, 113 bp, Artificial Sequence
  PCR amplified product
gtgagaaatgctgaggtcactgcagttgggtcaatggtcaggagacagtaaagaatttcatggaaagaagaagcctgtcagcagacttcaaaacaagtttgggcattatgacc

SEQ ID NO: 1151 - DNA, 116 bp, Artificial Sequence
  PCR amplified product
ggtttcatttgactgtaaagctgtatctccttctttcttggcatgtaaagatggcaggtggagcattctttgcctgctaccctctcccagccactctcatgttagtgaaagaatgg

SEQ ID NO: 1152 - DNA, 84 bp, Artificial Sequence
  PCR amplified product
gagaaatagccttcaaggagacagttcatgatagcttgctgtttaaagtgttcttatttaaattcccaaggatttcatgagaac